(19) United States
(12) Patent Application Publication
(10) Pub. No.: US 2010/0317534 A1
(43) Pub. Date: Dec. 16, 2010

(54) GLOBAL GERM LINE AND TUMOR MICROSATELLITE PATTERNS ARE CANCER BIOMARKERS

(75) Inventor: Harold R. Garner, Blacksburg, VA (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 12/814,294

(22) Filed: Jun. 11, 2010

Related U.S. Application Data
(60) Provisional application No. 61/186,745, filed on Jun. 12, 2009.

(51) Int. Cl. C40B 30/00; C12Q 1/68

(52) U.S. Cl. 506/7; 435/6

(57) ABSTRACT

The present invention includes a method of identifying an increase in microsatellite DNA from a genomic nucleic acid sample comprising: obtaining a microsatellite profile from a sample suspected of comprising cancer cells; comparing the microsatellite profile to a reference microsatellite profile from a reference genome; and determining in increase in the number of microsatellite DNAs from the sample as compared to the reference genome, wherein an increase in microsatellite DNA indicates a pre-disposition to cancer and the microsatellites are upstream from the estrogen receptor-related gamma gene (ESRRG).

### CROSS-REFERENCE TO RELATED APPLICATIONS

This application claims priority to U.S. Provisional Application Ser. No. 61/186,745, filed Jun. 12, 2009, the entire contents of which are incorporated herein by reference.

### STATEMENT OF FEDERALLY FUNDED RESEARCH

This invention was made with U.S. Government support under Contract No. 5-T32-HL07360-28 and P50CA70907 from awarded by the NIH. The government has certain rights in this invention.

### TECHNICAL FIELD OF THE INVENTION

The present invention relates in general to the field of cancer detection, and more particularly, to methods for detecting a predisposition to cancer as a result of microsatellite instability at the estrogen receptor-related gamma gene (ESRRG).

### BACKGROUND OF THE INVENTION

Without limiting the scope of the invention, its background is described in connection with cancer detection.

Excluding skin cancers, about 1.5 million new cancer cases occur each year in the United States and approximately 560,000 cancer-related deaths¹. Two major findings have changed the paradigm of cancer research and emphasized the need for molecular profiling of cancer: the discovery of predictive protein markers and genomic alterations in primary cancers²⁻⁴ and the development of targeting drugs, such as trastuzumab^{5,6} and the oral tyrosine kinase inhibitor, Lapitinib, that can induce remissions in HER-2 positive breast cancer patients with recurrent cancer^{7,8} and also decrease recurrences when used as an adjuvant therapy⁹.

While the complete etiology of epithelial-derived cancers is not yet known, several correlative genetic and environmental factors have been identified. One specific class of genetic events receiving increasing attention as both a marker and contributing factor of oncogenesis is microsatellite length mutations^{10,11}. Microsatellite repeats are ubiquitous and frequently polymorphic at rates that far exceed typical single-nucleotide mutation rates¹² in mammalian genomes, and their polymorphism can generate significant phenotype variation¹³⁻¹⁵. Somatic microsatellite length mutations are commonly observed in colorectal, endometrial, breast, and gastric carcinomas, and are a common feature of some lung cancers^{10,16,17}. Microsatellite instability (MSI), defined as extreme hypervariability of microsatellites throughout the genome, has been shown to be a manifestation of defects in DNA mismatch repair genes¹⁸. We hypothesize that both somatic and germ line microsatellite mutations may play an important etiological role in the development and progression of some cancers. It is critical to have knowledge of their mutational frequency, complexity, and diversity among different types of epithelial-derived cancers, as well as an understanding of how they vary in different normal genetic backgrounds.

### SUMMARY OF THE INVENTION

The present invention includes methods and kits for the detection of cancer. The invention can use a a custom oligonucleotide array to measure global microsatellite content (hybridization intensities representing the summation of all individual simple repeat-containing loci) among individual genomic DNA samples. Using this novel array, a unique and reproducible pattern of 26 differential microsatellites that specifically characterized breast cancer, colon cancer, and childhood hepatoblastoma patient germ lines was found. This same microsatellite hybridization intensity pattern was also detected in the tumor DNA of these same cancer patients, but not in DNA samples from healthy volunteers. These results indicate that some cancer patients might possess variable microsatellites that are predictive of future cancer development. Based on subsequent evaluation of individual loci containing array-identified differential motifs, we sequenced the 5′ UTR of the estrogen-related receptor gamma gene in ˜450 patient and volunteer samples and identified 5 to 21 copies of the (AAAG)ₙ repeat that was statistically significant for differentiating the germ lines of breast cancer patients from those of healthy volunteers. Our results indicate that microsatellite instability is complex, pervasive, and an antecedent to oncogenesis.

In one embodiment, the present invention includes a method of identifying an increase in microsatellite DNA from a genomic nucleic acid sample comprising: obtaining a microsatellite profile from a sample suspected of comprising cancer cells; comparing the microsatellite profile to a reference microsatellite profile from a reference genome; and determining in increase in the number of microsatellite DNAs from the sample as compared to the reference genome, wherein an increase in microsatellite DNA indicates a pre-disposition to cancer and the microsatellites are upstream from the estrogen receptor-related gamma gene (ESRRG). In one aspect, the microsatellite is TTTC and its copy number is elevated in the sample. In another aspect, the sample is from a patient suspected of having a pre-disposition to breast, colon or lung cancer.

In another embodiment, the present invention is a method of detecting exposure of cells to carcinogens or mutagens comprising: obtaining a microsatellite profile from a genomic nucleic acid from a cell sample suspected of exposure to the carcinogen or mutagen; comparing the microsatellite profile of the cell sample to a reference cellular microsatellite profile normal cell sample; and determining an change in the number of microsatellite DNAs from the cell sample as compared to the normal cell sample, wherein an change in microsatellite DNA indicates exposure to the carcinogen or mutagen. In another aspect, the cell sample is a clinical sample. In another aspect, the microsatellite profile is obtained using a microarray that comprises at least 3, 5, 7, 10, 12, 15, 18, 20, 22 or 25, spots selected from TTTC, ACCTGA, AAAGAC; AATTT; AATT; AATTAG; ATAATT; AAATTT; AAATTG; AAAATT; ACATTT; AAAACG; AAAACT; ACTTAC; AAAAAT; AAAAGT; AAT; AAAGTT; ATATA; AAATAT; AAAGAT; AATAAG; AATAGG; AAATAG; AAAATG; AACCTT; AATATT; AAAGGT; and AAAG. In another aspect, the method further comprises the step of knocking-down or knocking-out one or more genes in the cell sample and determining the change in microsatellite profile to identity one or more microsatellite sequences and the one or more genes that are adjacent to the change in microsatellite copy number to identify a suspected link between the microsatellite copy number and the one or more genes. In another aspect, a change in the copy number of the ACCTGA microsatellite is indicative of exposure to a carcinogen or mutagen.

Yet another aspect of the present invention includes a method of identifying a microsatellite associated with a disease condition from a sample comprising: determining whether one or more microsatellite sequences from the sample has increased upstream from the ESRRG as compared to the reference genome that comprise a change in the copy number of the microsatellite sequence. In another aspect, the method further comprises the step of knocking-down or knocking-out one or more genes in the cell sample and determining the change in microsatellite profile to identity one or more microsatellite sequences and the one or more genes that are adjacent to the change in microsatellite copy number to identify a suspected link between the microsatellite copy number and the one or more genes.

In yet another embodiment, the invention includes a method of identifying a patient with a predisposition to cancer comprising: determining if there is an increase or decrease in microsatellite copy number upstream of the AAAG tandem repeat locus located in the 5′ UTR of the estrogen-related receptor gamma gene (ESRRG) in a patient sample, the patient having the disease condition, wherein an change in microsatellite copy-number indicates a pre-disposition to cancer.

In yet another embodiment, the invention includes a method of identifying the phylogeny of a sample comprising: obtaining a microsatellite profile for the sample using a microarray that comprises 1-mers to 6-mers of: perfect repeats, single mismatches, double mismatches and single nucleotide deletions; comparing the microsatellite profile to a microsatellite profile from a reference genome; and determining the phylogeny of the sample based on a comparison of the microsatellite profile of the sample to the reference genome. IN one aspect, the sample is an unknown animal sample. In another aspect, the sample is a forensic sample.

Yet another embodiment of the invention is a nucleic acid microarray for the detection of microsatellites in a genome comprising: a substrate; and a plurality of groups of sample spots arranged in a two-dimensional array, wherein the plurality of sample spots formed in a predetermined positional relationship with each other, wherein the sample spots comprise 1-mers to 6-mers of: perfect repeats, single mismatches, double mismatches and single nucleotide deletion spots. In one aspect, the microarray comprises at least two 3- to 6-mers selected from AAAGAC; AATTT; AATT; AATTAG; ATAATT; AAATTT; AAATTG; AAAATT; ACATTT; AAAACG; AAAACT; ACTTAC; AAAAAT; AAAAGT; AAT; AAAGTT; ATATA; AAATAT; AAAGAT; AATAAG; AATAGG; AAATAG; AAAATG; AACCTT; AATATT; AAAGGT; and AAAG. In another aspect, the microarray comprises 53,735 unique probes. In another aspect, each of the probes is replicated three to seven times. In another aspect, the microarray further comprises all known transcription factor binding sites, ultra-conserved sequences, positive and negative controls. In another aspect, the array comprises at least 1,000 different oligonucleotides attached to the first surface of the substrate. In another aspect, the array comprises at least 10,000 different oligonucleotides attached to the first surface of the substrate. In another aspect, the microarray comprises at least 3, 5, 7, 10, 12, 15, 18, 20, 22 or 25, spots selected from AAAGAC; AATTT; AATT; AATTAG; ATAATT; AAATTT; AAATTG; AAAATT; ACATTT; AAAACG; AAAACT; ACTTAC; AAAAAT; AAAAGT; AAT; AAAGTT; ATATA; AAATAT; AAAGAT; AATAAG; AATAGG; AAATAG; AAAATG; AACCTT; AATATT; AAAGGT; and AAAG. In another aspect, the solid phase support is made of material selected from the group consisting of glass, plastics, synthetic polymers, ceramic and nylon.

The present invention also includes an array for identifying an increase in microsatellites in a polynucleotide sample from a patient suspected of having cancer, the array comprising: a substrate; and a plurality of groups of sample spots arranged in a two-dimensional array, wherein the plurality of sample spots formed in a predetermined positional relationship with each other, wherein the sample spots comprise 1-mers to 6-mers of: perfect repeats, single mismatches, double mismatches and single nucleotide deletion spots, the array comprising two or more microsatellite spots comprising AAAGAC; AATTT; AATT; AATTAG; ATAATT; AAATTT; AAATTG; AAAATT; ACATTT; AAAACG; AAAACT; ACTTAC; AAAAAT; AAAAGT; AAT; AAAGTT; ATATA; AAATAT; AAAGAT; AATAAG; AATAGG; AAATAG; AAAATG; AACCTT; AATATT; AAAGGT; and AAAG.

Another embodiment is a kit for identifying microsatellite variations in polynucleotide sample as compared to at least one reference sample, comprising: a substrate; and a plurality of groups of sample spots arranged in a two-dimensional array, wherein the plurality of sample spots formed in a predetermined positional relationship with each other, wherein the sample spots comprise 1-mers to 6-mers of: perfect repeats, single mismatches, double mismatches and single nucleotide deletion spots; reagents suitable for a labeling of the polynucleotide sample; and reagents for binding the labeled sample to the array.

Another embodiment is a method of identifying a microsatellite DNA that correlated with a disease condition comprising: obtaining a microsatellite profile from a genomic nucleic acid from a patient sample, the patient having the disease condition; comparing the microsatellite profile of the patient to a reference microsatellite profile that is obtained from a normal sample for a person that does not have the disease condition; and determining an change in the number of microsatellite DNAs from the patient sample as compared to the normal sample, wherein an change in microsatellite DNA indicates a pre-disposition to the disease.

### BRIEF DESCRIPTION OF THE DRAWINGS

For a more complete understanding of the features and advantages of the present invention, reference is now made to the detailed description of the invention along with the accompanying figures and in which:

FIG. 1: Comparison of normalized and log transformed signal intensity values for two individual cancer-free volunteer blood samples, before and after EBV-transformation (abscissa and ordinate, respectively), confirms the specificity of the array and its sensitivity to oncoviral contamination. The only motif that was statistically significant and reproducible for both samples was GAGCAG, labeled in blue, a repetitive motif found in the EBV genome. Each blue circle represents the comparative (primary vs. transformed) signal intensity for an individual probe, and the 5 probes collectively represent the GAGCAG motif family (i.e., all 5 possible cyclic permutations: GAGCAG, AGCAGG, GCAGGA, CAGGAG, and AGGAGC). Each probe intensity value represents the compendium of all loci in the analyzed genome that harbor the specific microsatellite sequence. The only substantial difference between the two genomes shown (primary and EBV-transformed blood from the same individual) is contributed by a single GAGCAG-containing locus in the latent Epstein Barr virus epigenome. The grey dots represent the remaining non-differential probes, out of a total of 5,356 motif permutations that include every possible microsatellite motif with a core repeat unit of 1-6 nucleotides. The R² value (excluding the GAGCAG motif family) was 0.97;

FIGS. 2A-2F: Comparison of normalized signal values for primary tumors breast cancer (BC) and colon cancer (CC) patients, matching patient B-lymphocytes (BC and CC germ lines), and blood samples from 6 ‘normal’, cancer-free volunteers reveals a consistent pattern of microsatellite motif changes. Each point on the scatter plot is the comparative signal intensity values for each perfect-match microsatellite probe on the array, and the signal for each microsatellite motif permutation is a summation of all genomic loci that contain that specific motif. Those microsatellite motif permutations that are statistically significant and reproducible across all cancer patient samples, compared to healthy volunteers, are labeled in color and noted. For example, the AAT microsatellite motif, along with its two cyclic permutations (ATA and TAA), are shown as purple triangles. There are 14,460 genomic loci containing the AAT motif, and each signal value for a probe representing an AAT permutation (purple triangles) results from the additive hybridization of all of fluorescently labeled DNA sequences. As with gene expression arrays, signal intensities do not behave perfectly linearly, but a larger intensity value in one sample versus another implies a higher [global] copy number for that sequence. The grey dots represent the remaining non-differential motifs and their cyclic permutations, out of a total of 5,356. Also noted in color is poly A/T, because the standard clinical test for microsatellite instability is measurement of 5 intergenic poly A sequences (Bethesda markers). However, we detected no variation in the global content of poly A/T;

FIGS. 3A-3F: Comparison of normalized signal values for childhood hepatoblastoma tumor (H) patients, matching patient B-lymphocytes, a small cell lung carcinoma (SCLC) cell line (H2141) and its matching EBV-transformed B-lymphocytes (BL2141), and blood samples from 6 ‘normal’, cancer-free volunteers also exhibited a consistent, specific pattern of motif changes. Those motifs that are statistically significant and reproducible across all samples are labeled in color and noted. (More detailed explanations of the meaning and significance of colored shapes are provided in the legend for in FIGS. 2A-2F). The grey dots represent the remaining non-differential motifs, out of a total of 5,356. Also shown are Poly A/T, which did not globally differ between samples, and the EBV-specific GAGCAG motif including all cyclic permutations, which was detected only in transformed cell lines;

FIG. 4: Hierarchical clustering of 26 cancer-specific motifs differentiates healthy volunteers from breast, colon, and childhood hepatoblastoma tumors. Clustering was performed using CLUSFAVOR 6.0 on normalized and log transformed signal ratios. Normal male and female volunteers are labeled N1-3 and N4-6, respectively, and cell lines are labeled in accordance with accepted nomenclature. Hepatoblastoma tumor and germ lines are labeled as H1T-H3T and H1G-H3G, respectively. Similarly, breast cancer patient tissues are labeled as BC1T-10T and matching blood as BC1G-10G. DNA extracted from primary colon cancer and matching germ lines are labeled as CC1T-3T and CC1G-3G, respectively. Note that cancer-free volunteer samples clustered apart from all cancer patient tumors and all but one of the cancer patient germ line samples. Most notably, non-small cell lung cancer cell lines and two breast cancer and matching blood cell lines clustered with cancer-free volunteer samples, whereas the three colon cancer cell lines (HCT15, HCT116, and RKO), the small cell lung cancer cell line (H2141) and one of the breast cancer cell lines (HCC1395) clustered with cancer patient samples. Bright red indicates the highest normalized intensity value, bright green indicates the lowest, and black represents median values;

FIG. 5: Plot of AAAG copy number (ordinate) for the longest allele for 6 sample types (abscissa), grouped as follows: healthy volunteers without family history (in 1° or 2° family members) of breast cancer, healthy volunteers with a breast cancer family history (see Supplementary Table 3 for specifics) of breast cancer, breast cancer (BC) patients, patients with colon polyps, and colorectal cancer (CC) patients. Designation of alleles as “short” or “long” is indicated by the blue horizontal line (alleles above the line have 13+ copies of AAAG and are designated as “long”). Note the lower incidence of the “long” allele in cancer-free volunteers (far left) and much higher incidence of the “long” allele in breast cancer patients (middle);

FIGS. 6A-6F: Global microsatellite pattern for the HCC1395 breast cancer cell line resembles that of primary breast cancer patients. Various views of the comparison of normalized signal values for breast cancer (HCC1395, HCC1187, and HCC2157) cell lines, matching blood cell lines (BL), and non-transformed B-Lymphocytes obtained from cancer-free volunteers are shown. Those motifs that were statistically significant and reproducible across primary cancer patient tumors are labeled in color and noted. The grey dots represent the remaining non-differential motifs, out of a total of 5,356. As shown, only HCC1395, a triple negative for ER, PR, and HER-2, and its matching blood line exhibited the pattern detected in samples obtained from primary cancer patients. The EBV-specific GAGCAG motif including all cyclic permutations, detected only in transformed cell lines, is also shown;

FIGS. 7A-7F: Global microsatellite content of colon cancer cell lines but not non-small cell lung cancer (NSCLC) cell lines recapitulates what was observed in primary patient tumors. Various views of the comparison of primary colon cancer tumors and germ liens, colon cancer cells lines (RKO, HCT15, and HCT116), NSCLC (H1437 and H2887) and matching blood (BL) cell lines, and non-transformed B-Lymphocytes obtained from cancer-free volunteers are shown. Those motifs that were statistically significant and reproducible across primary cancer patient tumors and also H2141 (SCLC cell line) are labeled in color and noted. The grey dots represent the remaining non-differential motifs, out of a total of 5,356. As shown, these cell lines did not exhibit the pattern detected in samples obtained from primary cancer patients. The EBV-specific GAGCAG motif including all cyclic permutations, detected only in transformed cell lines, is also shown;

FIG. 8: PAX2 can bind directly to the AAAG sequence in the 5′ UTR of ERR_γ. The AAAG repeat sequence (highlighted in red) and 100 by flanking sequences were examined using the Transfac database and TFSEARCH tool. BLAST scores and e values were 44.1-22.3 bits and 1e-07-1.7, respectively. The MATCH search was set to minimize the sum of both error rates, and results scores varied from 85.5 to 100. The THSEARCH scoring equation is based on a weighted sum and does not reflect statistical significance;

FIGS. 9A and 9B: A polymorphic AAAG repeat in 5′ UTR of ERR-γ is expanded in some cancer cell lines. A quick gel survey of the ERR-γ locus was followed by sequencing of each of the PCR products. (4b) The expected product size of the PCR amplicon was 369 bp. PCR amplicons show that all cancer free humans samples (H1-17) possess 7-10 tandem copies of AAAG within the 5′ UTR of the ERR-γ gene (18q21.2), while breast cancer 2 and 3 (BC2 and BC3, HCC2157 and HCC1187 cell lines, respectively) with their matched blood lines (B2B1, B3B1), as well as colorectal cancer 3 (CC3, RKO cell line) are heterozygous at the loci, with upper bands ranging from 19-21 repeats. To validate polymorphism specificity in human disease, a series of animal controls were also used: M=mouse, Ch=chimpanzee, G=gorilla and O=orangutan. (4c) The band for a cancer-free individual (N1) and upper/lower bands from a heterozygous breast cancer (BC) PCR sample were gel-purified and sequenced, confirming the normal 9 copies of the AAAG repeat and products of differing lengths in a heterozygous breast cancer sample. Samples details are provided as Supplementary Tables 1 and 6);

FIG. 10: Analysis of control probes indicates that the global microsatellite content array confirms binding specificity. Comparison of normalized signal values for probes representing wild-type (WT), single mismatch (SM), double mismatch (DM), and deletion (Del) probes for four representative microsatellite motifs and also the average of all motifs on the array was used as a measure of array specificity. The average signal intensities shown were calculated based on all cyclic permutations for the given motif for all 53 DNA samples hybridized to the array. The resulting averages are displayed on the ordinates, and the standard deviations are shown as error bars. Note that specificity decreases as alterations are made to the center nucleotide base, and standard deviations are lowest for perfect match (WT) probes. Comparisons were made for all microsatellite motifs represented on the array, and the four motifs shown were chosen to represent a broad range of intensity values. Note that all WT motif signals exceeded their corresponding mismatch probes, confirming binding specificity;

FIG. 11A: Colon cells exposed to MNNG (alkylating agent) for 72 hours

FIG. 11B: Detection of specific DNA damage after treatment with alkylating agents over time; and

FIG. 11C: Lung cancer patient DNA is compared to DNA from cancer-free volunteers. Distinct, reliable and reproducible patterns of DNA changes are detected within a single species, in this case, humans. Similar patterns measured for breast, colon, and childhood cancers, thus creating a universal signature for cancer.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Microsatellites are typically defined as tandemly repeated sequences (motifs) of one to six nucleotides that are very widely distributed throughout the genome and are frequently variable in the number of times the motif is repeated. Microsatellite alterations occur in most tumors, but their frequency and spectra are variable, with certain types of tumors (e.g., hereditary non-polyposis colorectal cancers) harboring significantly elevated rates of mutation at these loci¹⁹. The recurrence of microsatellite mutations in several loci in multiple different cancers, including known tumor suppressor genes (e.g. PTEN), is strong evidence that these microsatellite mutations are indeed important events in the progression of these cancers. Even stronger evidence lies in the observation that there is likely some selection for these specific mutations, because microsatellite mutations in other loci with similar repeat sequences are not observed in these tumors²⁰. Alterations in repeat unit number in and around coding sequences can have important quantitative and qualitative effects on gene expression²¹⁻²⁴ and thus could potentially contribute directly to cancer progression. Elucidation of the nature and cause of microsatellite mutations in cancer and how they are distinct from those operating in the germ line can provide critical insights into the molecular underpinnings of the oncogenetic process. Furthermore, an investigation of global microsatellite differences in various cancers might provide cancer-specific signatures, as well as help identify individual cancer biomarkers.

To investigate microsatellites on a global scale, our laboratory designed a custom array that measures genomic microsatellite content, similar to a comparative genomic hybridization array (aCGH). The array probe design was based on computationally-derived simple repeat DNA sequences (i.e. all possible 1- to 6-mer microsatellite motif combinations, including every cyclic permutation and corresponding complement sequence), not on unique sequences derived from any specific genome. Unlike aCGH array recorded hybridization intensities that are used to estimate copy variations at specific positions within the genome, the global microsatellite array is used to directly compare intensity values that represent the summation across all individual microsatellite motif-containing loci. For example, the intensity recorded on the probe for the AATT motif (and probes for its cyclic permutations, ATTT, TTTA, and TTAA) measures the contributions from the 886 AATT motif specific microsatellite loci spread throughout the reference human genome. The global microsatellite array can therefore be used to specifically and accurately measure significant motif-specific variations (polymorphisms), whether they are in the germ line or arise as somatic mutations, in any DNA sample. This allowed us to perform, for the first time, a thorough and unbiased analysis of cancer genome microsatellites, which led to the discovery that germ line microsatellite variability might represent a cancer predisposition biomarker.

Global microsatellite content distinguishes three different cancer types. Genomic DNA samples were acquired from 6 cancer-free volunteers (blood), 5 patients with expression microarray-confirmed²⁵ basal-type breast cancer (breast tissue and blood), 5 patients with luminal-type breast cancer (breast tissue and blood), 3 colon cancer patients (colon tissue and blood or unaffected tissue), 3 children with hepatoblastoma tumors (liver tissue and blood), 3 pairs of breast cancer and matching blood cell lines, 3 pairs of lung cancer and matching blood cell lines, and 3 colon cancer cell lines (Table 2). Each of these 53 genomic DNA samples was subsequently co-hybridized with the same human DNA standard (derived from a mixed population of male and female donors) to a custom oligonucleotide array that measures summated global microsatellite content. After verification of data quality, statistical analyses were performed, and only those motifs with signals that were reproducible for replicate sequences and also biological replicates were considered in further analyses. Statistical significance (one-way ANOVA, with Benjamini & Hochberg corrected p value <0.05) was required for each differential motif, and consistency for cyclic permutations was additionally required in order to consider each differential motif as robust.

Sample acquisition and preparation: Genomic DNA was extracted from blood samples collected from volunteers (Tables 2 and 7) by the McDermott Center for Human Growth and Development Genetics Clinical Laboratory in accordance with Institutional Review Board (UTSW IRB#1287-355). Most cell lines were provided by Drs. Girard, Minna, and Boothman. Patient samples were provided by Drs. Perou, Tomlinson, Lewis, and the UTSW Tissue Repository, with each institution's review board approval. All other genomic DNA was purchased from Coriell Cell Repositories (Camden, N.J.) or American Type Culture Collection (Manassas, Va.).

To measure array specificity, a custom 70-mer oligonucleotide (SEQ ID NO.: 1) (5′-GCAAAGGGACCCACGGTGGAACAGGAGCAGGAGCAGGAGCGGGAGGGGCAGGAGCAGGAG-3′) and its complement were designed based on the GAGCAG repeat-containing EBV sequence. The custom 70-mers were de-salted, annealed, and PAGE-purified by the manufacturer (Integrated DNA Technologies, Coralville Iowa), and 500 pmoles was spiked into a cancer-free volunteer DNA sample (N4, Table 2).

Array design, manufacture, and processing: Each array consisted of 53,735 unique probes, each replicated 7 times (for a total of 376,145 probes/features) at different positions across the array, including 14,634 probes to measure repetitive DNA sequences for all possible 1-mers to 6-mers (5,356 perfect repeats (WT), single (SM) and double (DM) mismatches and single nucleotide deletion (DEL) probes). Also included on the array were all known transcription factor binding sites (2005 Transfac database), ultra-conserved sequences⁴⁵, RepBase sequences (Genetic Information Research Institute, 2005, www.girinst.org) and a series of controls. A database containing all raw array data from these experiments and a text file of the corresponding probe identifiers and sequences are available for download at http://discovery.swmed.edu/gmc.

All arrays were manufactured by Roche NimbleGen (Madison, Wis.) following their standard production methods for maskless photolithography, including additional internal controls. DNA (˜1 μg, 250 ng/μl) labeling, hybridization, and scanning were performed following their aCGH standard protocol. All test samples (labeled with Cy3) were co-hybridized with Cy-5-labeled Promega (Madison, Wis.) human reference DNA, and raw intensity values were provided via CD.

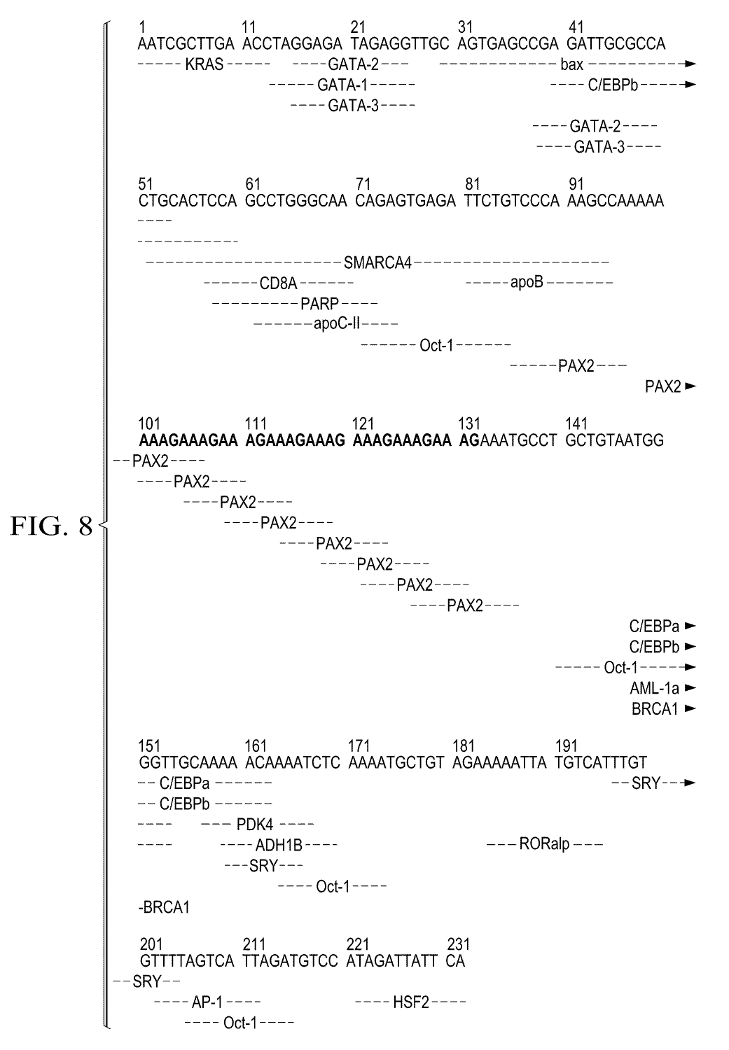

Array data processing and statistical analysis: Background subtraction and quantile normalization was performed across all arrays using NimbleScan software (Roche NimbleGen), followed by regression analysis to compare all reference sample signal intensity values (R²=0.93±0.06). To reduce the potential effect of outliers, only the median 5 probe values were considered for further analysis (i.e., maximum and minimum values were discarded for each set of replicate probes on each array). GeneSpring was used to perform additional normalization (percentile shift and baseline transformation), pairwise comparisons and one-way ANOVA with Benjamini & Hochberg (B-H) correction. For microsatellite motifs, any observed difference (≧2-fold, B-H. p value ≦0.05) was also expected to occur consistently across all possible cyclic permutations. Control probes were used to gauge background levels, reproducibility of reference samples, and final statistical output. As expected, the intensity values decreased predictably between microsatellite-specific control (WT, SM, DM, and DEL) probes (FIG. 8).

Computation of probe occurrences in genomes: Each of the 5,356 microsatellite probes on the array was also computationally aligned to the published human reference genome (NCBI Build Number 36, Version 3, Human Genome Sequencing Consortium release 4, Mar. 24, 2008). A Perl script was written to search for all 1-mer through 6-mer microsatellite motifs (minimum length of 18 bp). These microsatellites were loaded into a MySQL database and subsequently aligned to all exons, introns, and promoter regions (defined here as 1 kb 5′ of the start site) of the human genome to determine the number of occurrences in each of these regions of importance. The genetic regions were constructed by downloading the human Gene and Gene Prediction Tracks RefSeq table, March 2006 assembly, from the UCSC Genome Table Browser (genome.ucsc.edu).

All microsatellite occurrences were also aligned to the nearest SNP-associated comparative genomic hybridization value, as obtained from Illumina 109K SNP array (Illumina Inc., San Diego, Calif.) data for 10 breast cancer patients (Table 2) to determine the contribution of copy number variations to global microsatellite content. Global gain/loss in copy number, estimated as the average signal amplification ratio (tumor vs normal, diploid DNA) for all SNPs associated with each individual microsatellite locus compared to the number present in the reference genome, was negligible (˜2.6% variation on average) for microsatellite motifs determined to be differential using the custom microsatellite array.

Genotyping: Forward (SEQ ID NO.: 2) (5′ ACCTAGGAGATAGAGGTTGC 3′) and reverse (SEQ ID NO.: 3) (5′ CTTCTTCTGCACTATCAGGG 3′) primers were designed to amplify a 369 by length fragment of the ERR-γ gene including the 5′UTR AAAG repetitive sequence. PCR was performed using Promega 2×PCR Master Mix (Promega) per manufacturer instructions. Products were gel-purified using Qiagen gel extraction kit (Qiagen, Valencia, Calif.) and sequenced by the McDermott Center Sequencing Core Facility. Hardy-Weinberg equilibrium was tested using X² test of goodness of fit, with 1 degree of freedom, checking for long and short allele distribution (where “long” is defined as 13+ copies of the AAAG motif, and “short” is defined as fewer than 13 copies). Microsatellite instability (MSI) status was performed by McDermott Sequencing Core using the Promega MSI Analysis System, Version 1.2 (Table 3). MSI status was assigned according to the Bethesda Guidelines^{46,47}. To identify putative transcription factors, the AAAG-containing region of ERR-γ, including 100 bp flanking sequences, was searched against the Transfac database using BLAST, MATCH, and TFSEARCH tools⁴⁸.

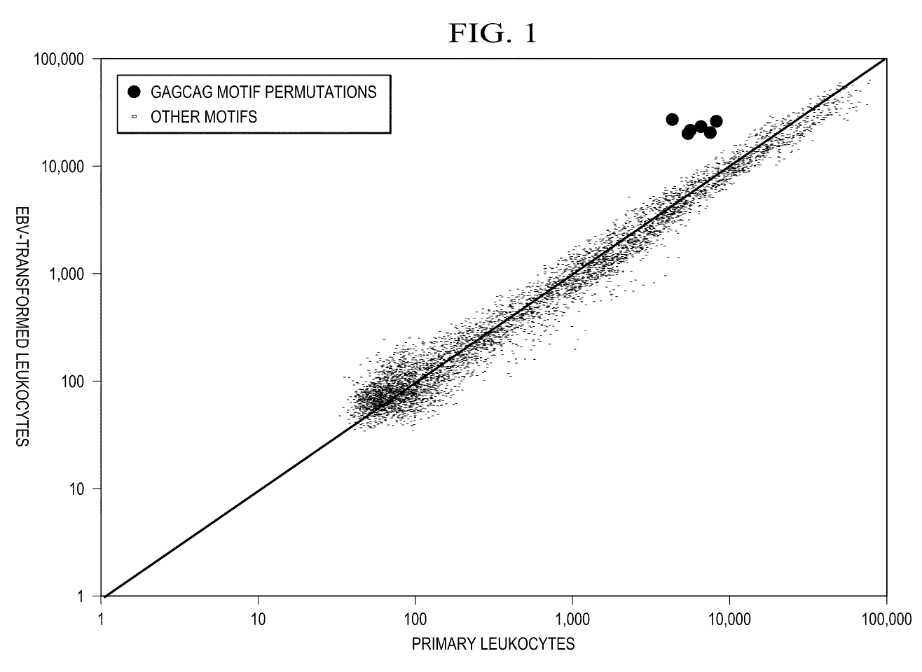

One motif, a GAGCAG repeat, was reproducibly observed as differential between cancer cell lines, which were spontaneously immortalized, and the matching B lymphocyte lines established through Epstein-Barr virus (EBV) transformation. The EBV virus contains a copy of this repeat, and to confirm that the array was specifically detecting the contaminating EBV epigenome, we compared DNA extracted directly from B lymphocytes and from a matching EBV-transformed cell line we established for two ‘normal’ samples. As shown in FIG. 1, GAGCAG motif permutations (shown as 5 blue circles) were the only differential probes detected between primary and EBV-transformed B lymphocytes, affirming array specificity and the value of EBV-specific GAGCAG motif permutations as an internal control. Likewise, spike-in of a custom 70-mer oligonucleotide (500 pmoles) including the GAGCAG motif and flanking EBV genomic sequence into a cancer-free volunteer DNA sample recapitulated the specific increase in the hybridization intensity of all 5 GAGCAG motif permutations (data not shown). It is notable that EBV transformation and subsequent culture of the cells did not significantly alter the host genomic microsatellite content (FIG. 1, grey dots), which was verified by regression analysis of each blood sample before and after EBV transformation (R²=0.96). For comparison, regression analysis of the human standard used on each of the arrays was R²=0.93±0.06 standard deviation. Because global microsatellite content was unchanged by transformation, we were able to also compare primary tissue and cell line-derived DNA samples.

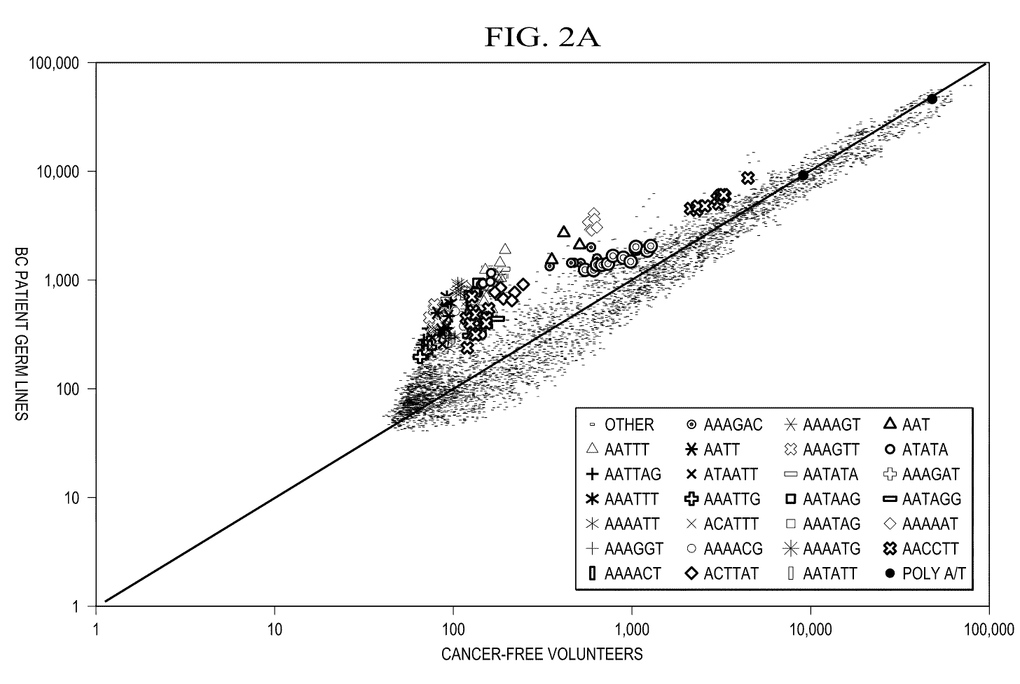

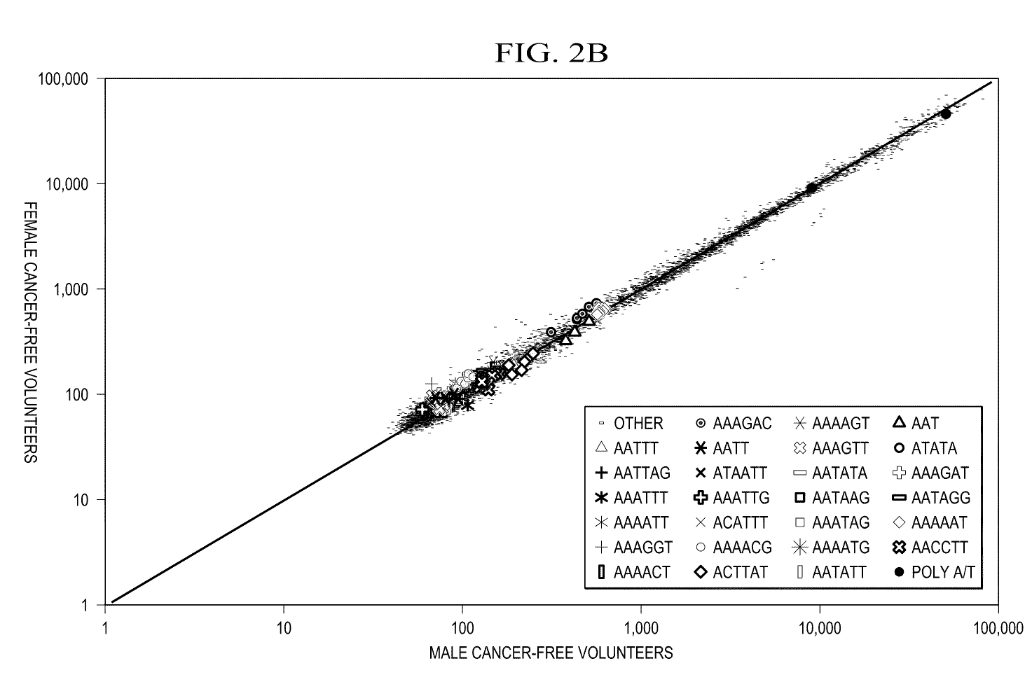

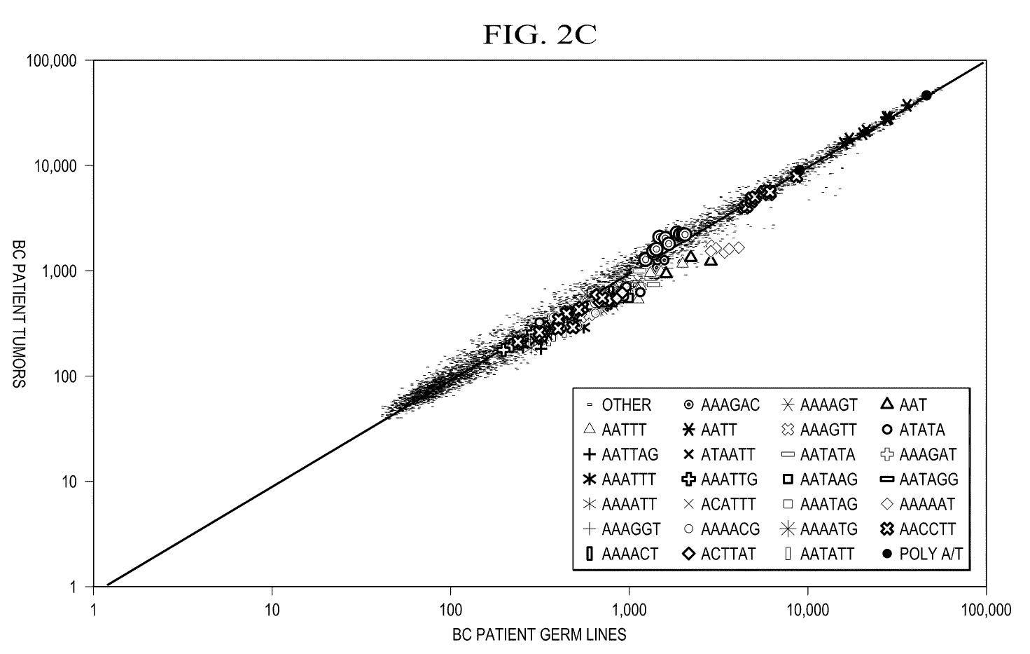

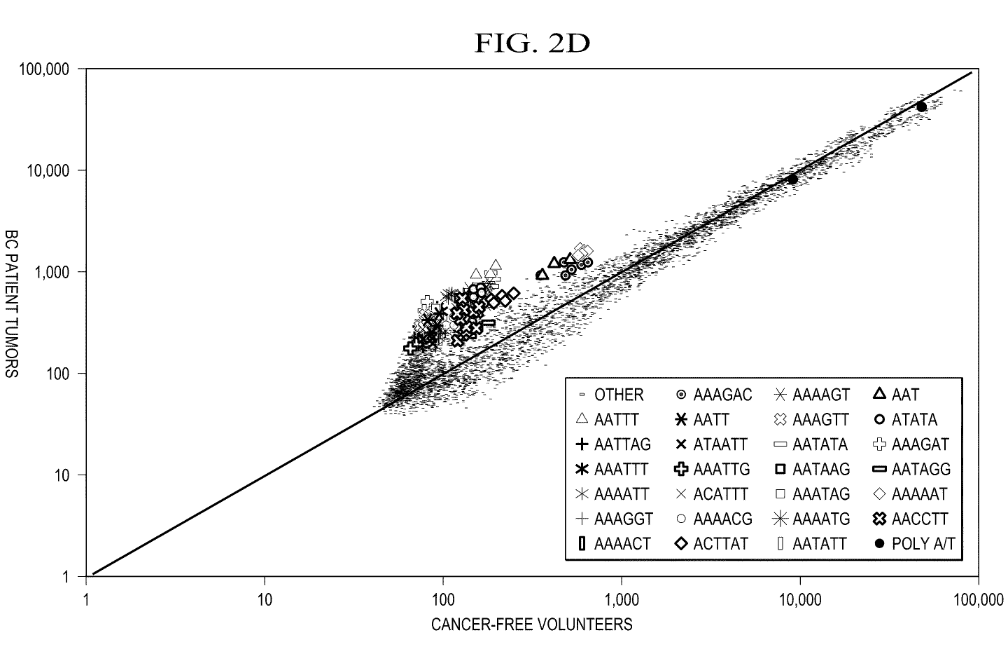

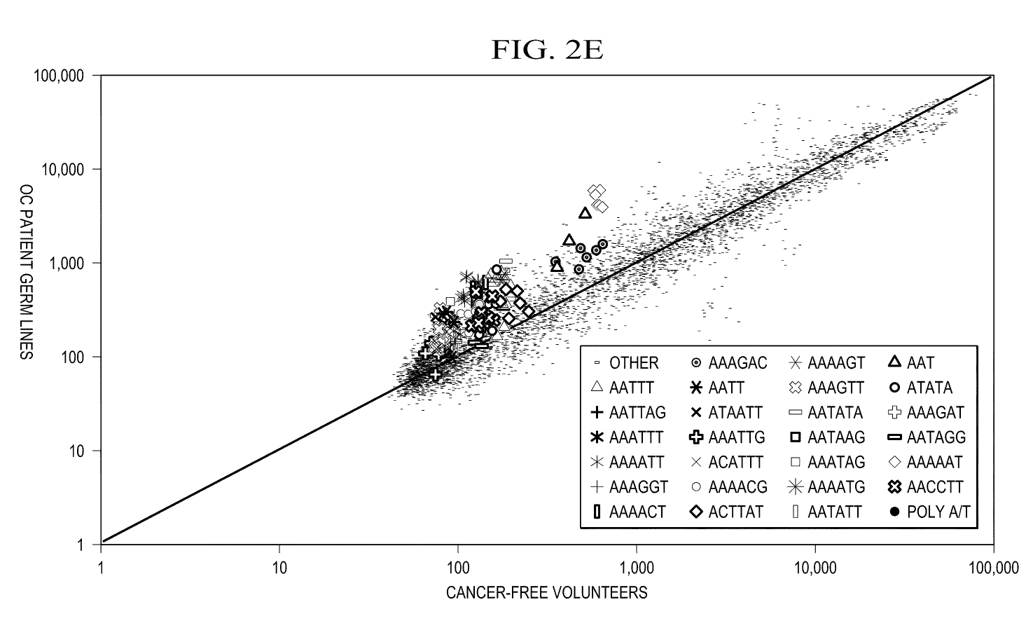

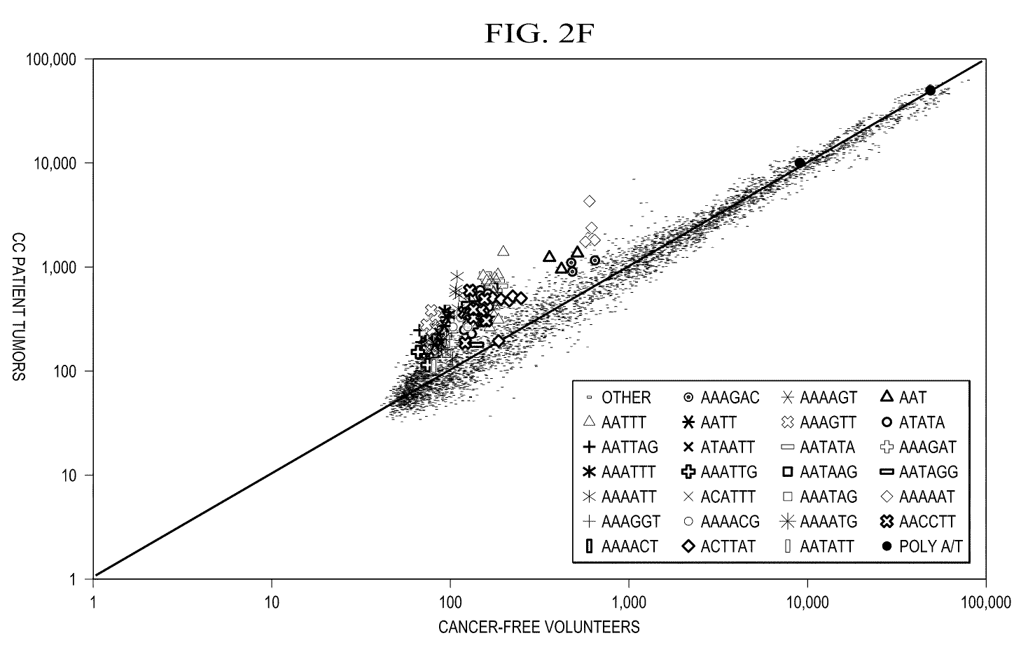

We next analyzed the various cancer patient and cancer-free volunteer samples, individually and in groups for statistical purposes. Based on analysis of the germ lines of 6 cancer-free volunteers (3 men and 3 women) versus 10 breast cancer patients (all women), there were 26 statistically significant microsatellite motifs (including cyclic permutations) that consistently differed between each cancer-free volunteer and all ten patient samples (FIG. 2A). When each patient germ line was examined separately (compared individually to each cancer-free volunteer sample, for a total of 60 pairwise comparisons), each of these 26 motifs, along with their cyclic permutations, were found to be differential. This was true for age and gender matched comparisons, indicating that gender and ethnicity were not factors related to the higher incidence of these global microsatellite motifs in the germ lines of breast cancer patients. A direct comparison of female and male cancer-free volunteers showed no differences in global microsatellite content, including the 26 cancer patient specific motifs (FIG. 2B).

Notably, very little difference was detected between the tumor DNA and matching germ liens of these same breast cancer patients when directly compared (FIG. 2C), although the 26 cancer patient-specific microsatellite motifs were detected as differential between breast cancer patient tumors and cancer-free volunteers (FIG. 2D). These results are consistent with the known heritability of breast cancer, which is estimated to range between 10% and 25%²⁶, and these 26 motifs could represent a breast cancer predisposition signature. The ten breast cancer patient tumors could be further divided into basal and luminal types (5 each), but a direct comparison of these tumor sub-types produced no statistically differential motifs (data not shown). Interestingly, while all 10 of these breast cancer patients exhibited this distinctive microsatellite motif profile in both their cancer tissue and germ line DNA (FIG. 2A to 2F), this same pattern was detected for only one out of the three breast cancer cell lines (i.e., HCC1395) tested (FIG. 1), including its matching EBV-transformed blood line (HCC1395BL). These results suggest that some cell lines may be more faithful than others at recapitulating the molecular characteristics of primary tumors.

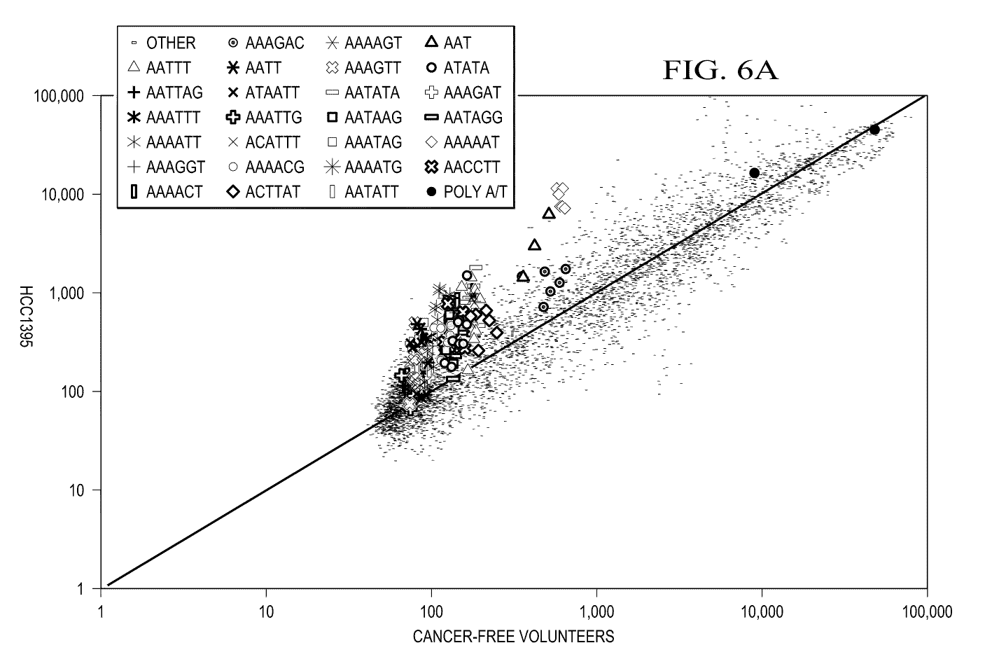

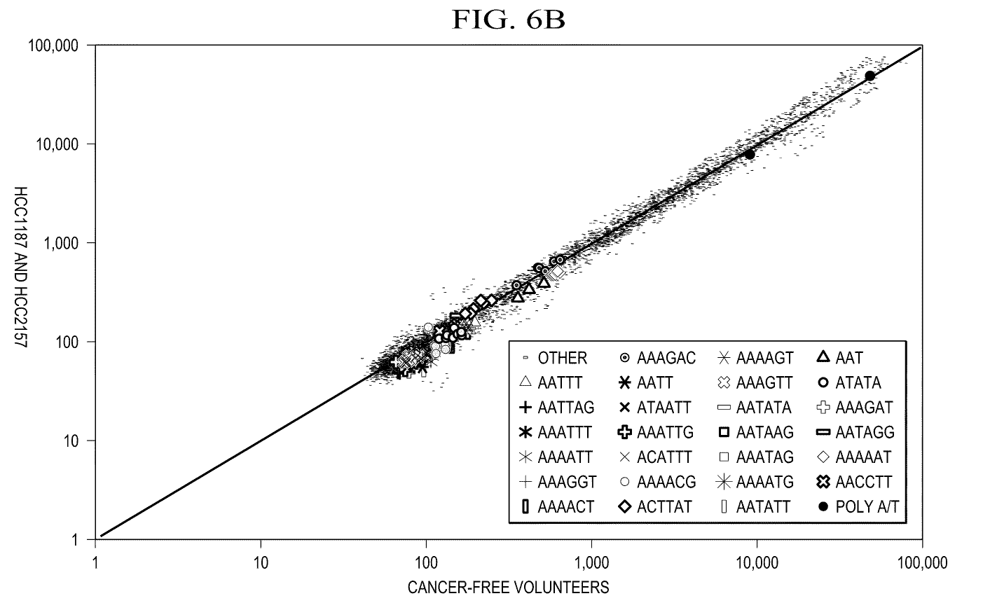

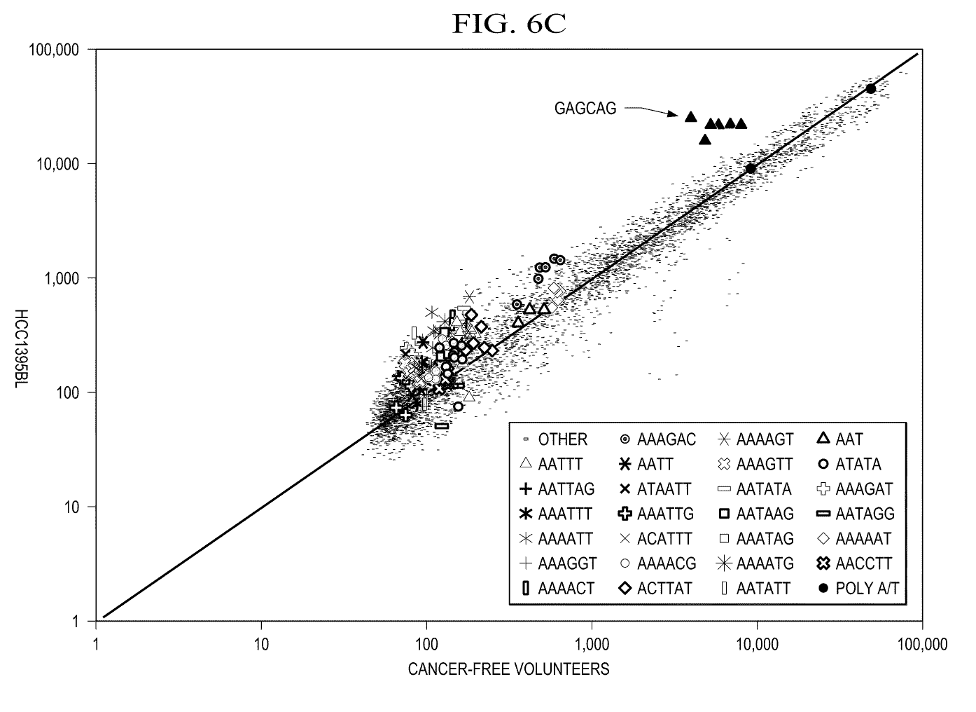

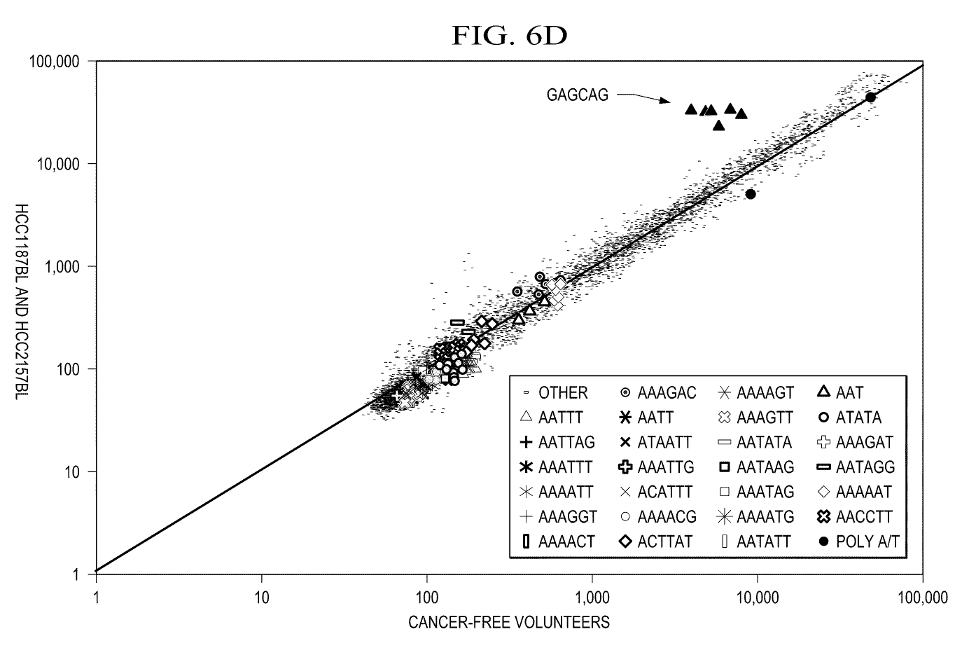

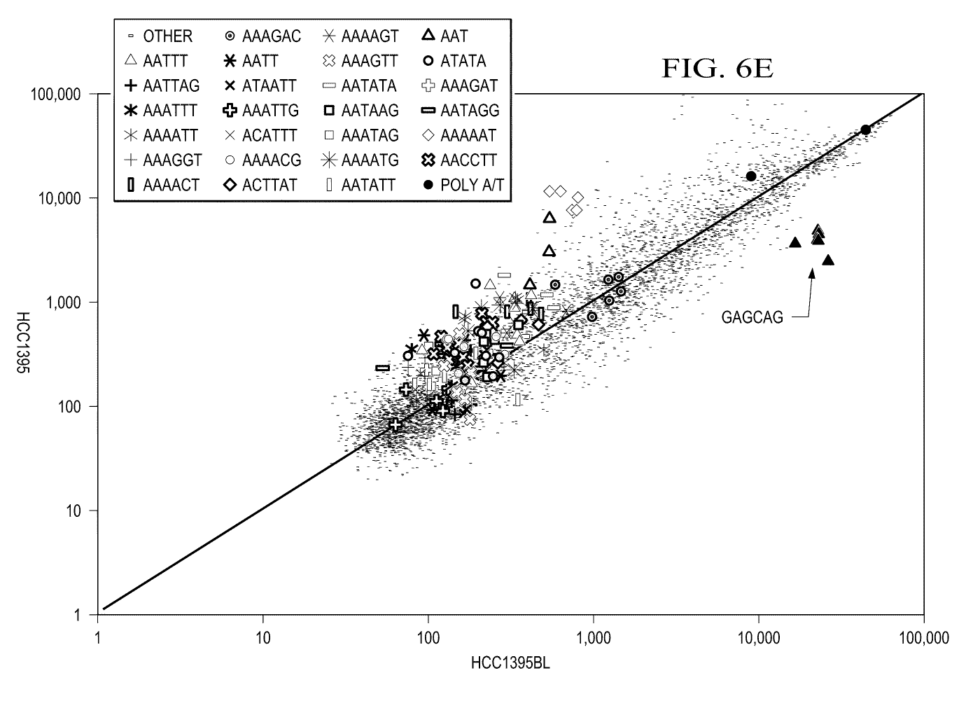

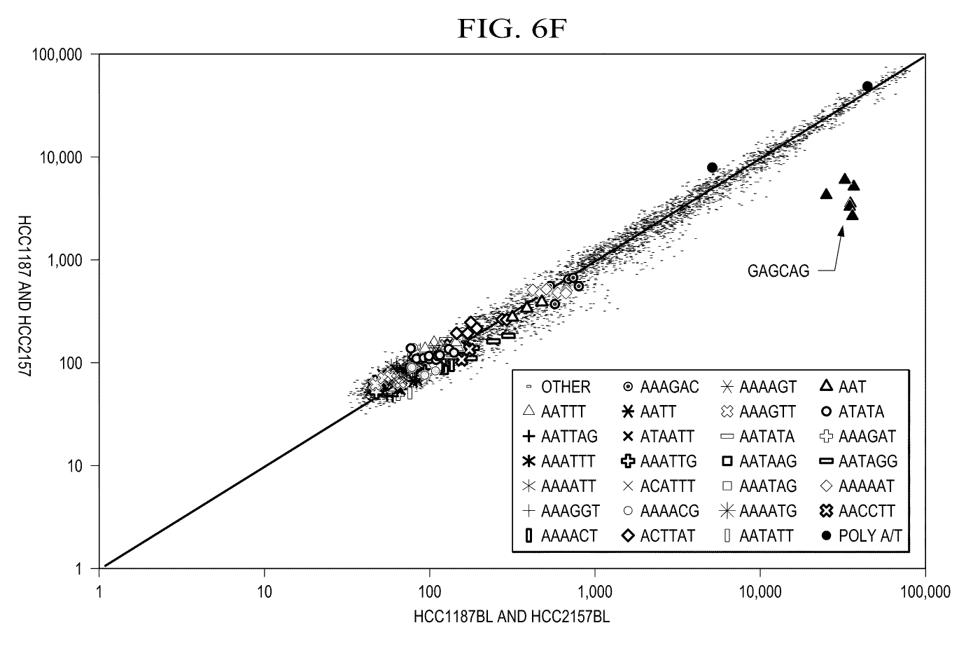

Examination of 3 colon cancer patients yielded similar results to what was observed for breast cancer patients, with a distinctive global microsatellite signature apparent between cancer patients and cancer-free volunteers. Specifically, all 26 motifs identified in breast cancer patients were also statistically significant (B-H p value ≦0.05, fold-change ≧0.05) and reproducible among colon cancer patient germ lines when compared to cancer-free volunteers (FIG. 2E), with the exception of one patient germ line sample that did not harbor the microsatellite pattern observed in the other two germ line samples. However, all 26 differential microsatellites were reproducibly differential among all three colon cancer patient tumors (FIG. 2F). Although there were observable differences between colon cancer patient tumors and matching germ lines (FIGS. 6A-6C), these differences did not include the canonical set of 26 motifs that characterized cancer patients from cancer-free individuals, again tracking what was observed for breast cancer patients. Matching normal DNA was not available for the colon cancer cell lines (RKO, HCT15, and HCT116) that were examined using the custom microsatellite microarray. However, each of these cancer cell lines resembled the primary cancer tumors (FIGS. 6D-6F).

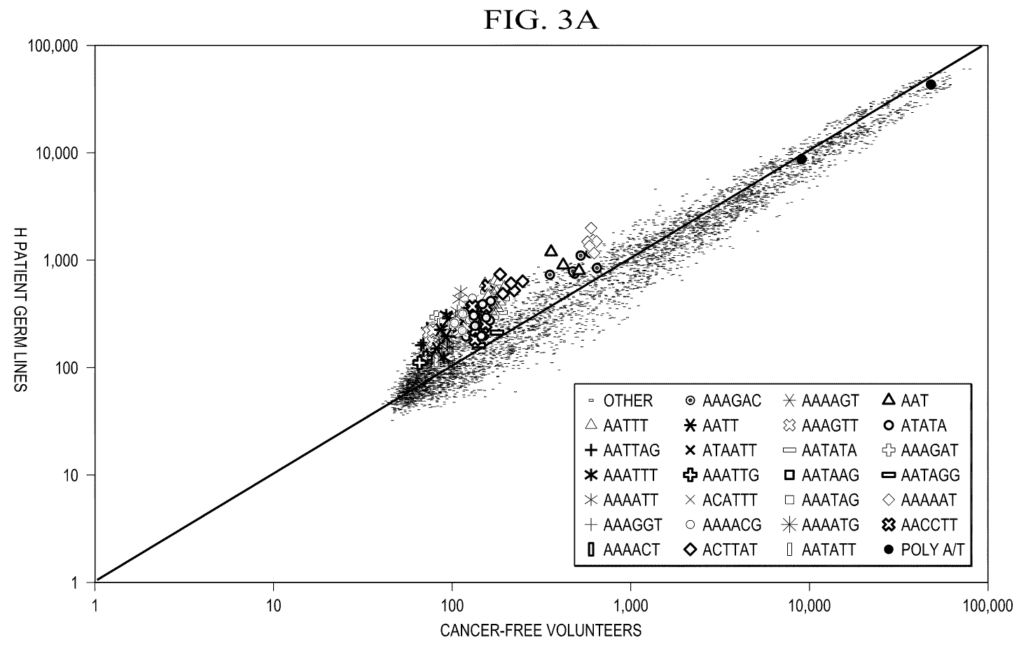

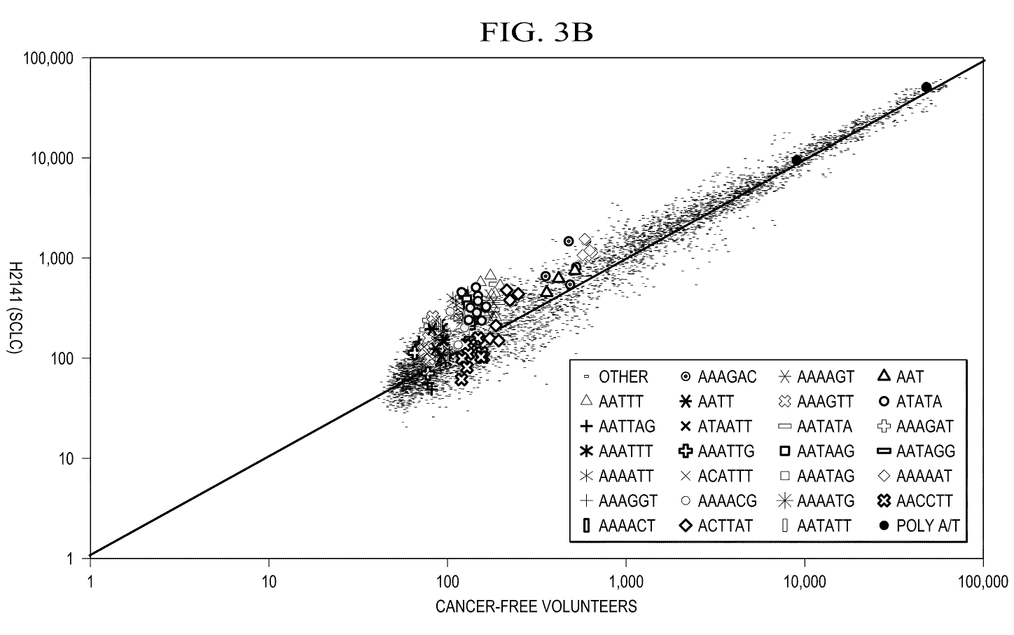

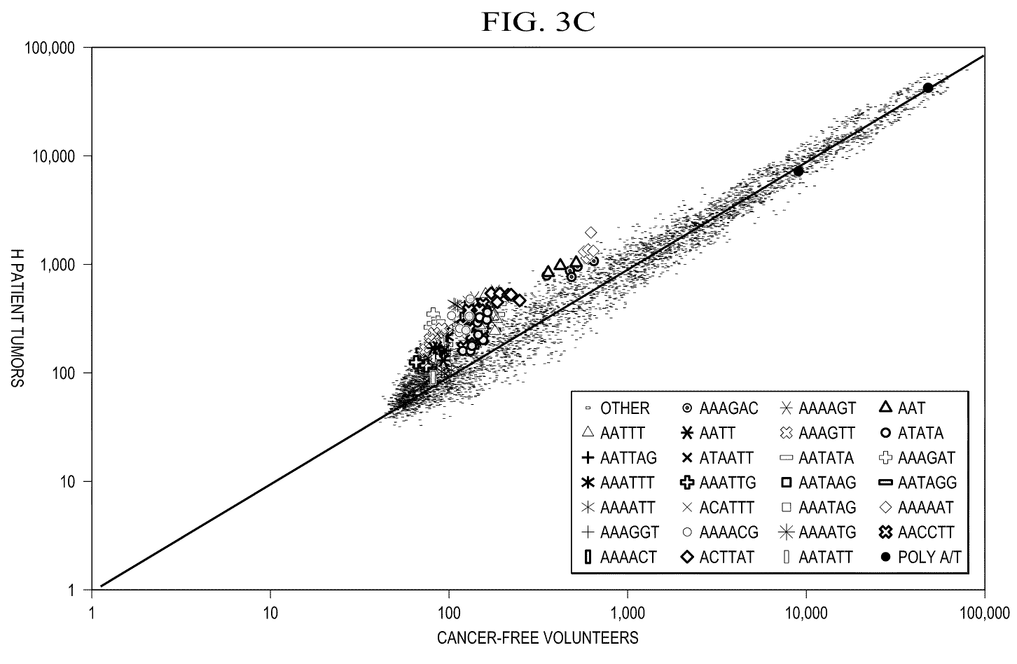

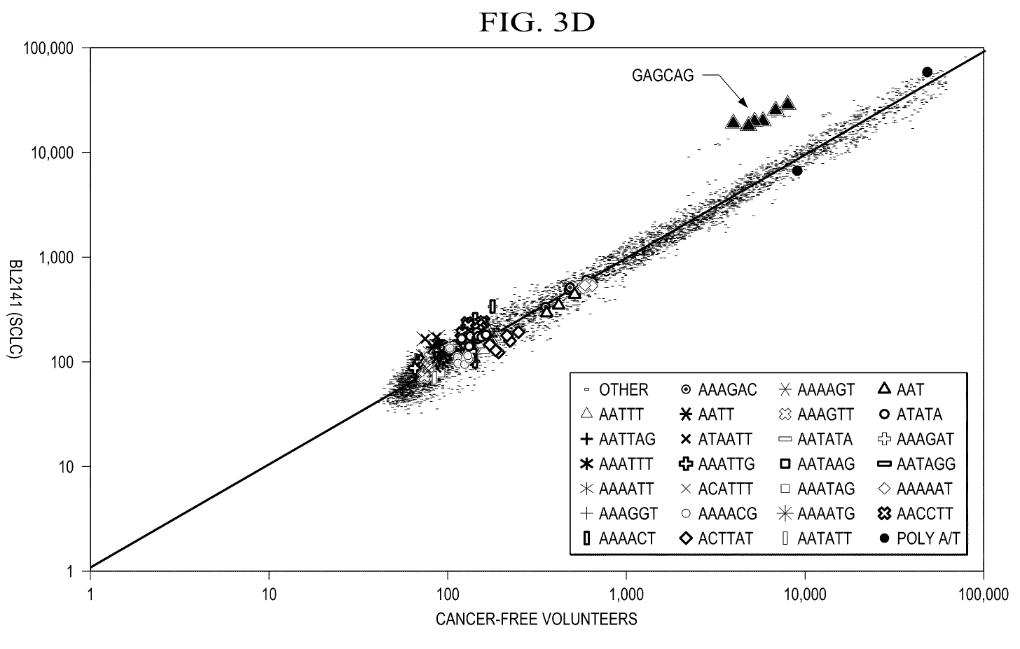

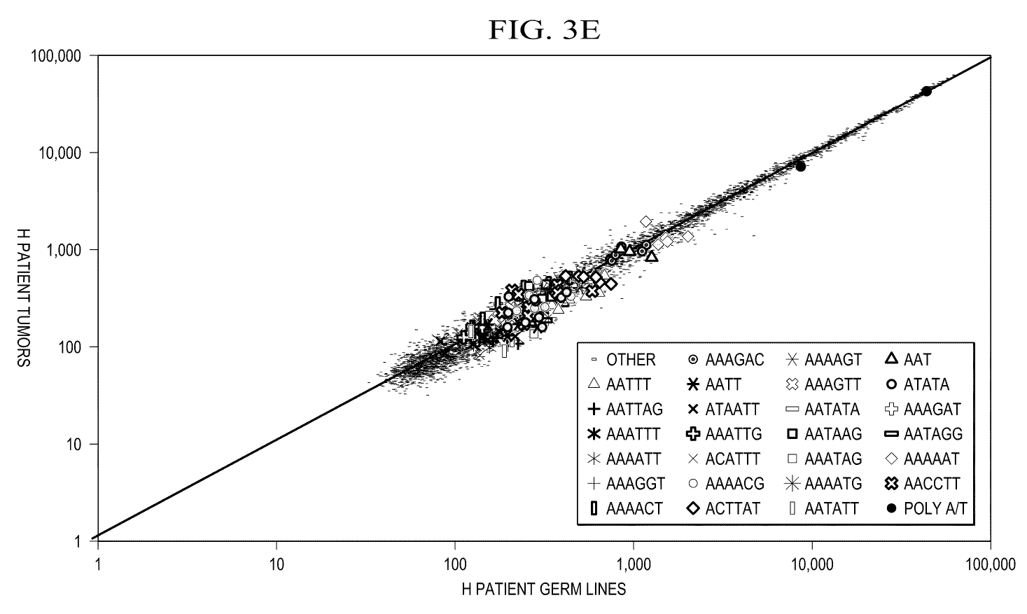

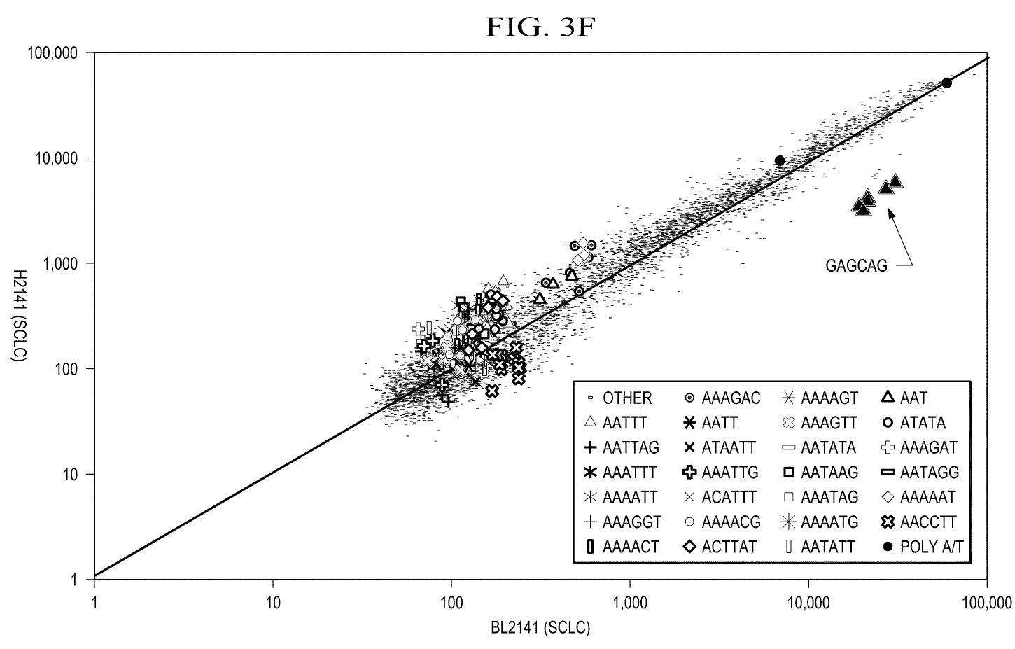

We next evaluated hepatoblastoma tumors from children, which should have a dominant genetic component given their early development, and found a global microsatellite pattern identical to what was observed in breast cancer patients (FIG. 3A-3F). The same 26 microsatellites that were identified in breast cancer and colon cancer samples were differential between cancer-free volunteers and both the germ lines (FIG. 3A) and tumors (FIG. 3C) of hepatoblastoma patients, and no microsatellite motifs differed between tumor and germ line DNA (FIG. 3E). Drastically different results were obtained for lung cancer cell lines, however, that were originally derived from smokers. Only the small-cell lung cancer cell line (H2141) exhibited the unique global microsatellite signature (FIG. 3B), with similar differences detected in the 26 microsatellite motifs determined to be differential in breast and colon primary cancer tissues and childhood hepatoblastoma tumors. The matching blood line (BL2141), on the other hand, was nearly identical to that of cancer-free volunteers (FIG. 3D); this finding is consistent with a neoplastic process resulting from exposure to an environmental carcinogen (i.e., patient was a smoker for 50-pack years, Table 2). The two non-small cell lung cancer lines and matching blood lines were also indistinguishable from cancer-free volunteers (FIGS. 6A-6F).

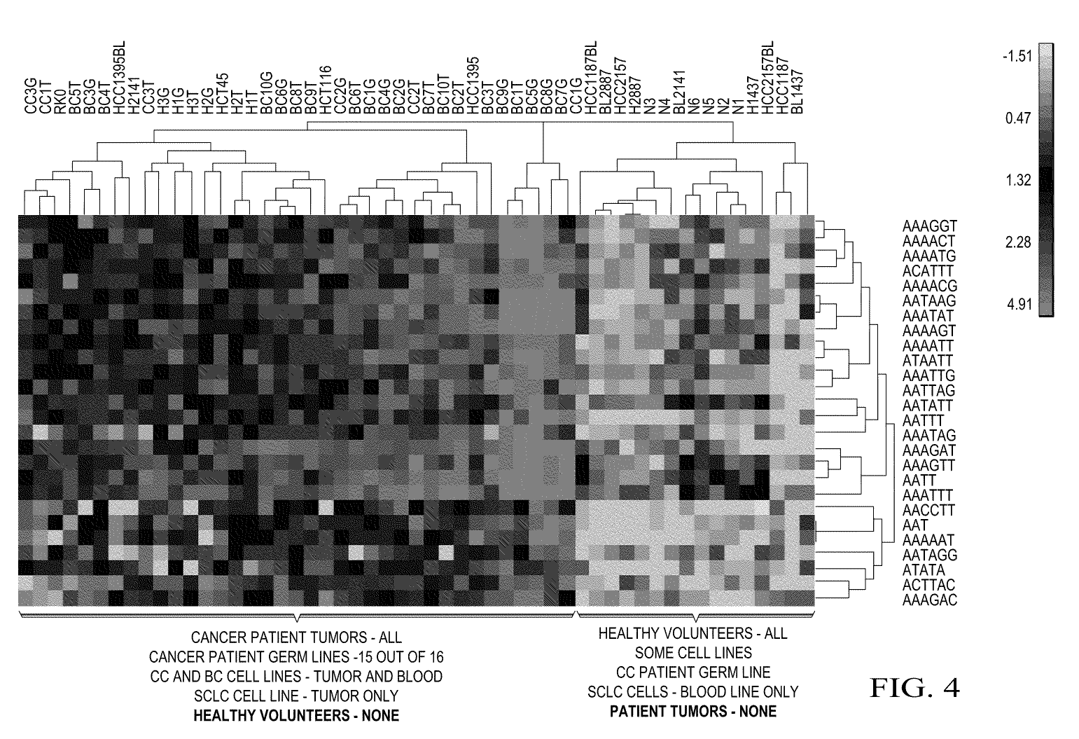

One-way ANOVA analysis of all samples followed by hierarchical clustering confirmed that a global microsatellite signature accurately separated all primary tumors from healthy volunteers samples (FIG. 4). In each of these cancers, the differential loci were members of families with similar motif patterns (i.e., A-T rich motifs), which may be a manifestation of disruption in the mismatch repair machinery or DNA replication process. Using the Promega MSI (microsatellite instability) genotyping kit, we confirmed that all three of the colon cancer cell lines were MSI-high (Table 3). This is in agreement with a previous report that these colon cancer cell lines were confirmed as MSI-high and carry truncating mutations in the p300 gene as a consequence of polymorphisms in two poly-A tracks and also coding SNPs²⁷. This extensively used ‘gold standard’ for classification of MSI is based upon the analysis of only 5 intergenic poly-A repeats, out of a total of 169,315 poly-A and poly-T repeats found within the genome sequence²⁸. However, it should be noted that in no case were any polynucleotide motifs, including poly-A and poly-T, observed to be differential in our data set, indicating that this test drastically underestimates the amount of global microsatellite mutation because it is not sampling those motifs that vary most significantly. Notably, breast cancer and colon cancer patient samples were not identified as MSI-unstable using the kit (Table 3), although we identified a global microsatellite signature similar to that observed for colon cancer cell lines using the custom microarray.

To determine if the increased incidence of microsatellites in cancer samples relative to cancer-free volunteers was a function of copy number changes in the genomic content, we analyzed whole genomic SNP array data on the twenty breast cancer patients for differences in regions containing microsatellites. The gains and losses for each microsatellite at each locus were calculated for each sample and subsequently compared. Based on this analysis, differences in variations in global microsatellite content as ascertained by the custom microsatellite array was not due to large gains or losses of chromosomal content. The contribution of segmental chromosomal duplications to the global microsatellite signature detected in breast cancer samples (compared to normal reference DNA) was negligible (less than 3% for all differential microsatellite motifs).

Identification of a putative predisposition biomarker for breast cancer and colorectal neoplasia: Based on the published human reference genomic sequence, the 26 cancer signature motifs are associated with a total of 42,702 loci, 27,578 of which are in close proximity (i.e., within 1,000 bp) to gene coding regions (Table 4). Although not included in the canonical set of 26 cancer-specific microsatellites, we chose the statistically significant but moderately differential AAAG motif to further investigate, due to smaller repeat unit size, which is an indication of a higher likelihood for polymorphism, its prevalence in the genome, and the number of genes that harbor the AAAG motif that are also implicated in cancer. For this motif, we found 14,311 copies in the entire genome, 4,127 of which are located within genes (exons, introns, UTRs, upstream and downstream areas). When limited to the 7,183 “cancer” genes (defined as those genes found in NCBI's EntrezGene using the search terms “cancer” and “tumor”), we found 128 in the 5′ UTR and 27 in the promoter region, which we defined as 1 kb upstream of those genes.

We prioritized each AAAG locus by copy number, which is positively correlated with a higher likelihood of being polymorphic²⁹ and subsequently designed and tested 28 PCR primer sets against a panel of 42 samples that included 12 cancer-free volunteers, 6 human diversity samples, 17 cancer cell lines, and a variety of controls. We found 11 of these loci to be polymorphic (i.e., 10 that exhibit different sizes and one that is frequently deleted) in the human samples (data not shown). Of the 11 polymorphic markers, two were of particular interest. One of the two markers containing an AAAG repeat, found in the TBL1Y gene located on the Y chromosome was absent in all female samples (data not shown). However, this microsatellite was also absent in some lung tumors but not in their matched B lymphocyte-derived cell lines, consistent with frequent deletion of the entire Y chromosome in some non-small cell carcinomas³⁰. The second interesting AAAG tandem repeat locus is located in the 5′ UTR of ERR-γ (estrogen-related receptor gamma, ESRRG, located on chromosome 1q41), which has 10 copies of the 4-mer (AAAG) motif, as found in the reference human genome sequence in the UCSC genome browser. ERR-γ is an orphan nuclear receptor and operates independently of estrogen; however, ERR-γ does bind to certain estrogen response elements to activate transcription³¹. Also, ERR-γ and its known co-activators have been linked to breast, ovarian and colon cancer³² and more recently to tamoxifen resistance in invasive lobular carcinoma of the breast³³.

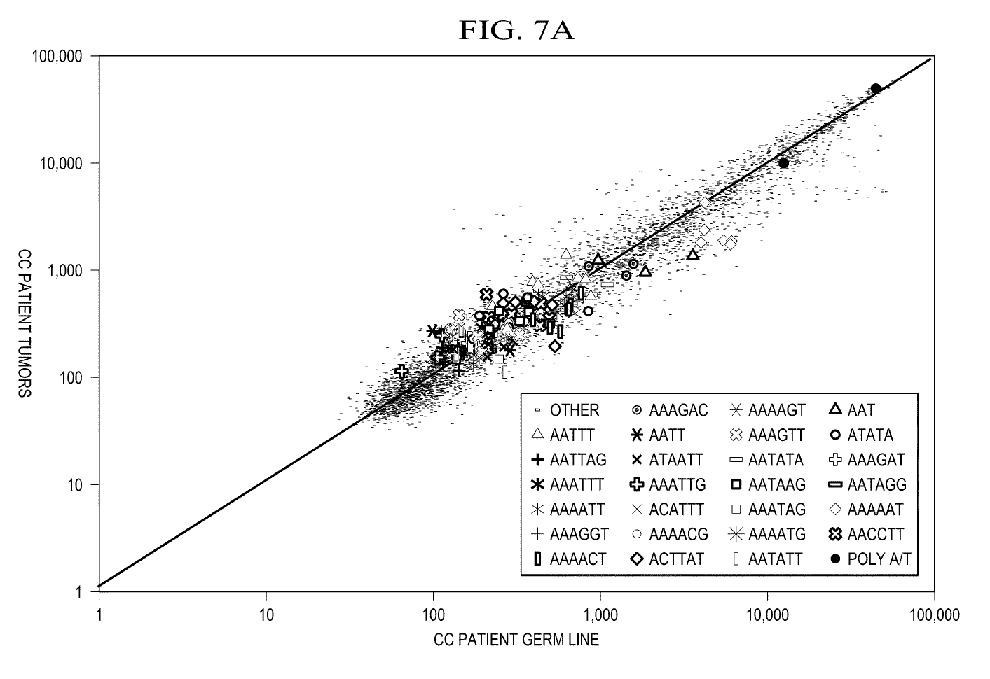

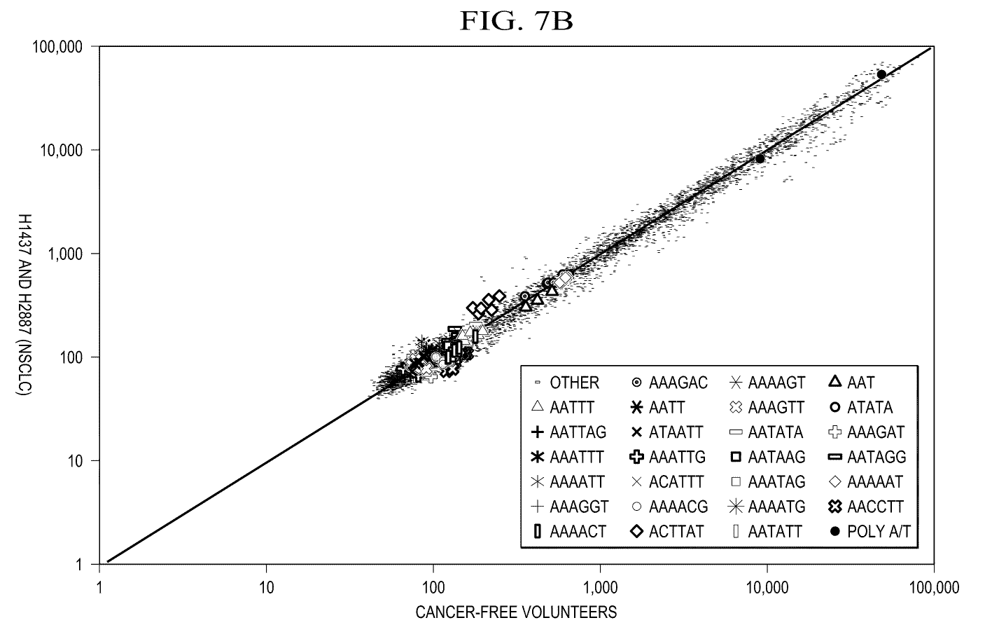

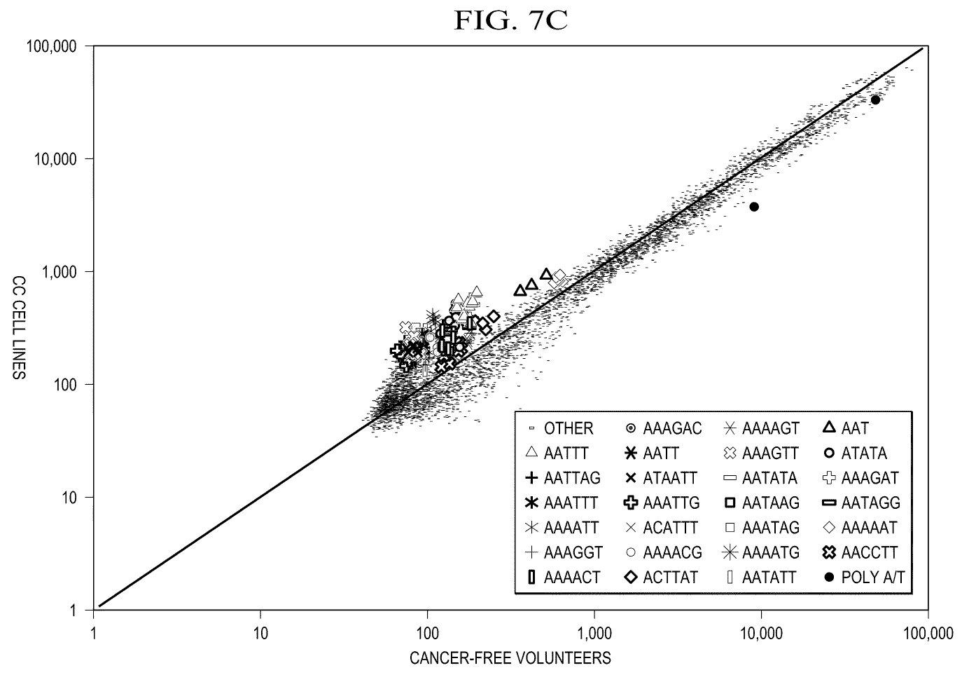

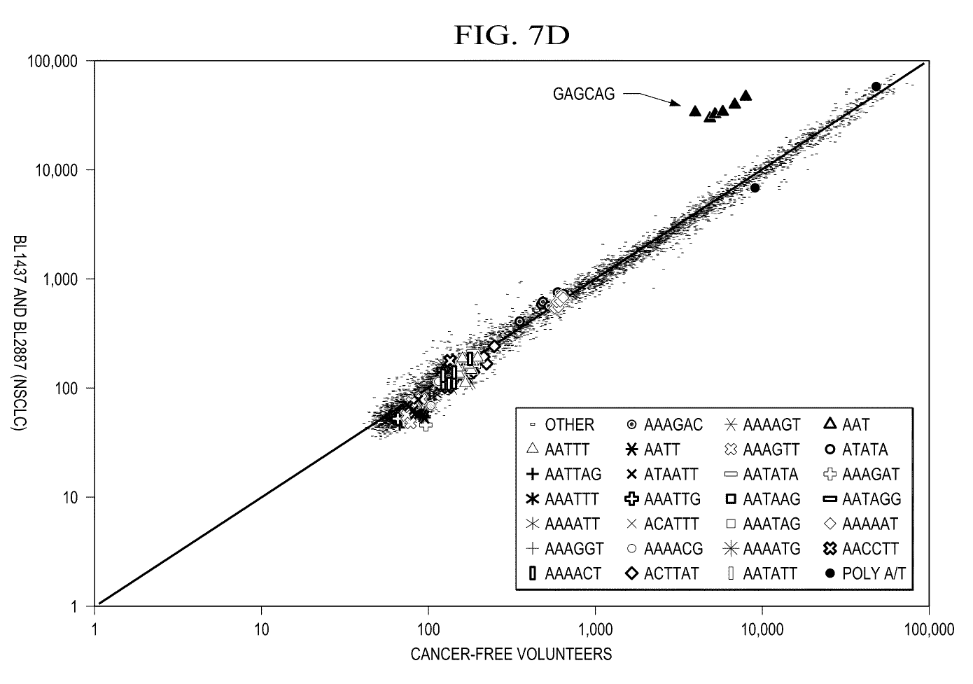

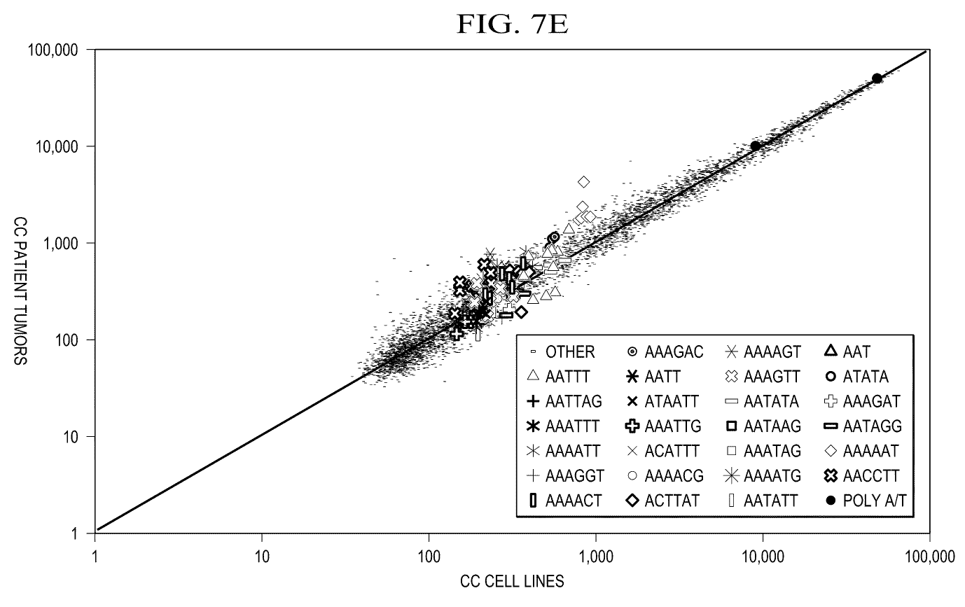

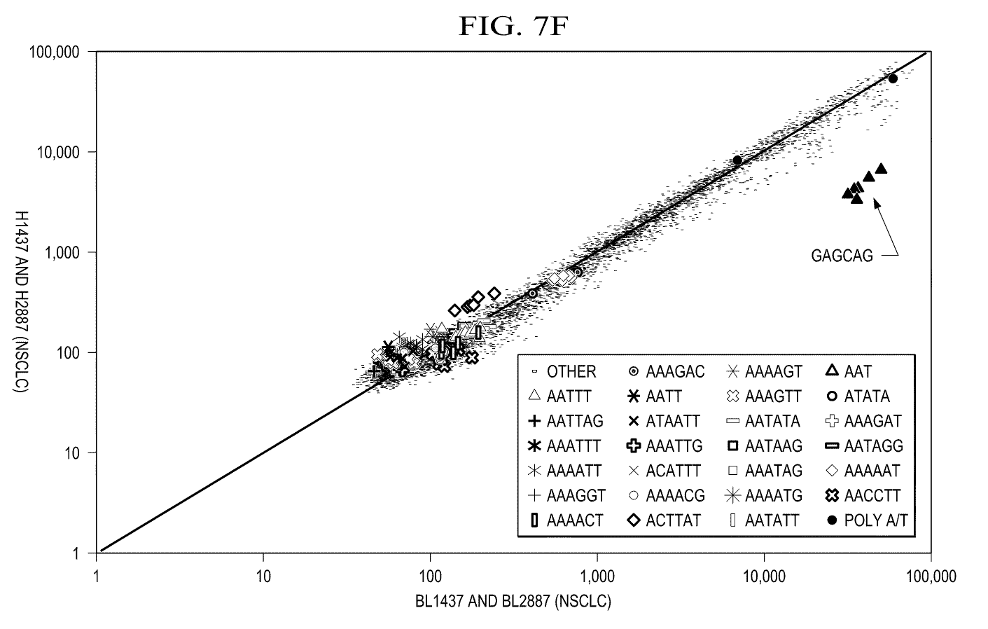

ERR-γ has 2 known isoforms, one with an alternative first exon and one with an alternative 5′ UTR. It is possible that the differential AAAG microsatellite confers alternate regulation of ERR-γ, as is thought to be the case for the gene encoding the parathyroid hormone receptor, which also harbors a polymorphic (AAAG)ₙ repeat sequence in its promoter region that co-varies with adult height³⁴. There are 22 candidate transcription factors (FIGS. 7A-7F) that could potentially bind to the region of the 5′UTR of ERR-γ containing the AAAG repeat (the repeat itself plus 100 by flanking sequences), one of which (paired box gene 2, PAX2) is capable of binding the repeat unit itself.

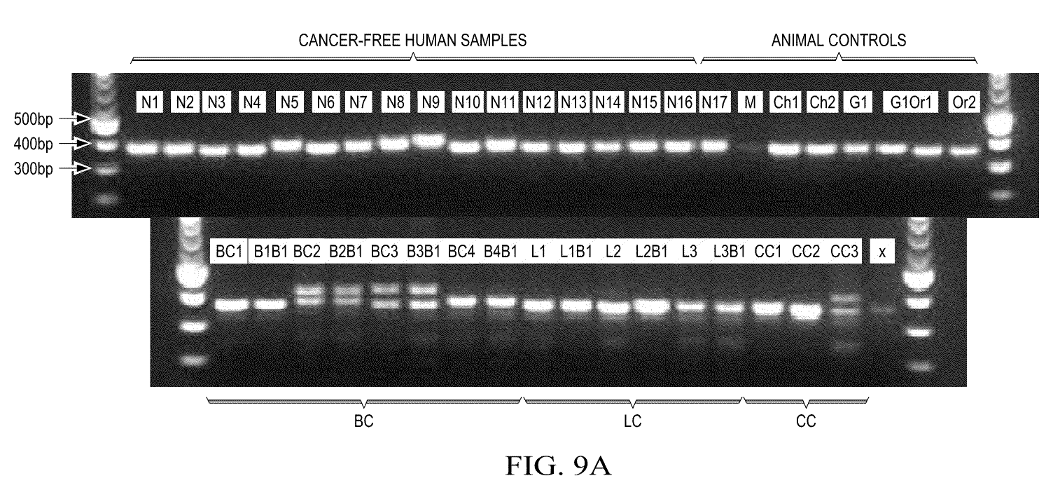

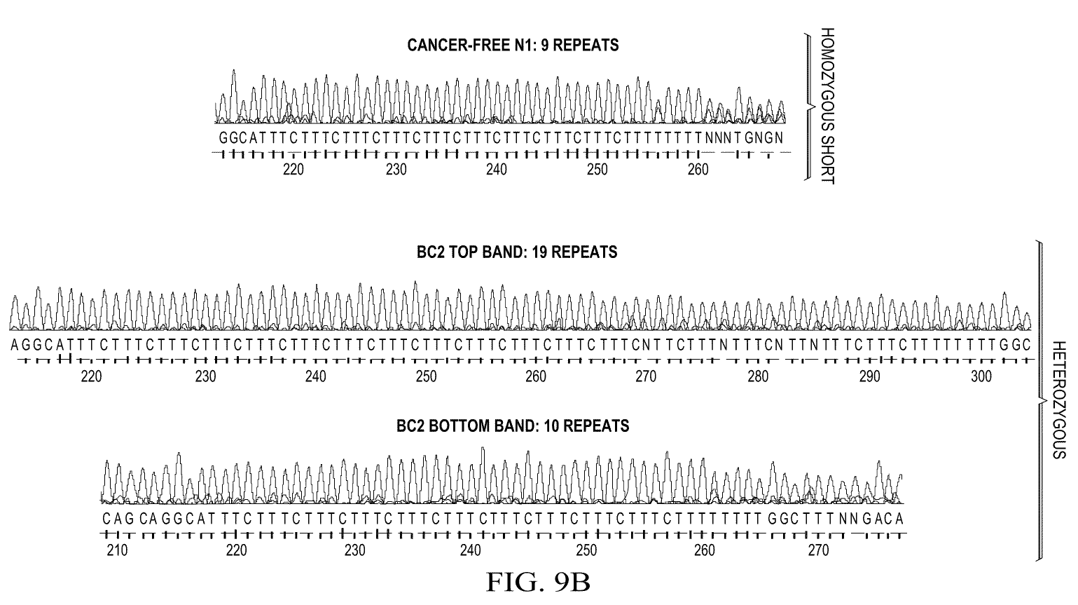

As shown in FIG. 9A, two of the four breast cancer cell lines were heterozygous at the ERR-γ (AAAG)ₙ locus, as were the matched blood lines and one of the colon cancer cell lines. Sequencing of the 42 samples indicated that homozygous samples carry a short version of the microsatellite, which ranges between 7 and 12 repeat units, and heterozygous samples carry one short copy and one longer allele ranging from 13-21 repeat units (FIG. 9B). The frequency of this variation was then measured by sequencing this locus in an expanded set of 447 samples, including 147 breast cancer patients, 104 patients with colon neoplasia, 22 lung cancer cell lines, and 174 cancer-free volunteers with and without a family history of breast cancer.

Based on genotyping results, the size of the AAAG motif ranged between 5 and 21 copies. We chose 13 motif copies as the cut-off length for classification as “long”, as this number was the most rare among samples (only one patient with an allele of this length), and 12 copies was relatively common and equally observed (4-6 incidences) for each class of sample (e.g., cancer and non-cancer). Based on these criteria, carriers and non-carriers of the longer allele for each category of patient are presented in Table 1.

TABLE 1
Summary of the Incidence of the ERR-γ Repeat in Patient Samples

| | | | | | Statistics | |
|---|---|---|---|---|---|---|
| | | | | | (p value) | |
| | | | | | Baseline Group | |
| | | | | | Healthy: no | |
| | | | | | BC family | Healthy: |
| | Non- | | | | hx | all |
| | carriers | Carriers | Totals | Incidence | n = 125 | n = 174 |
| Healthy volunteers: | | | | | | |
| No BC family hx | 119 | 6 | 125 | 4.8% | — | 0.7992 |
| BC family hx | 45 | 4 | 49 | 8.2% | 0.4705 | 0.5143 |
| Cancer patients: | | | | | | |
| Breast cancer | 126 | 21 | 147 | 14.3%* | 0.0134 | 0.0130 |
| Colorectal cancer | 45 | 6 | 51 | 11.8% | 0.1086 | 0.2100 |
| Other sample types: | | | | | | |
| Colorectal polyps | 48 | 5 | 53 | 9.4% | 0.3072 | 0.3504 |
| Lung cancer cell lines | 21 | 1 | 22 | 4.5% | 1.0000 | 1.0000 |
| Totals | 404 | 43 | 447 | 9.6% | 0.1040 | 0.1498 |
| Additional groupings: | | | | | | |
| All healthy volunteers | 164 | 10 | 174 | 5.7% | 0.7992 | — |
| Colon cancer + polyps | 93 | 11 | 104 | 10.6% | 0.1289 | 0.1622 |
| Breast + colon cancer | 171 | 27 | 198 | 13.6%* | 0.0132 | 0.0143 |

Note:
“BC family hx” refers to 1° or 2° family members with breast cancer.
“Carriers” refer to persons in which the long allele (defined as at least 13 copies of the AAAG motif) is present.
Asterisk indicates a statistically significant difference.
BC = breast cancer;
hx = history.
A detailed list of patients and genotyping information is provided as Supplementary Table 4.

As shown, a statistically significant higher incidence of long allele carriers (p value=0.0134, two tailed Fisher's exact test) was observed for breast cancer patients (14.3%), compared to healthy volunteers (4.8%), which translates to a relative risk ratio of 2.97 (14.3/4.8). A similar trend was observed when cancer-free volunteers were compared to patients with colon neoplasia (11.8% and 9.4% long allele carriers for persons with colorectal cancer and colon polyps, respectively), although this difference was not statistically significant (p value=0.129, two tailed Fisher's exact test). However, comparison of cancer-free volunteers with breast and colon cancer patients combined (i.e., both sets of cancer patients considered as one group) did yield statistically significant results (p value=0.0132, two-tailed Fisher's Exact test). The percentage of carriers for the 22 lung cancer cell line samples examined was similar to what was observed for cancer-free carriers (4.5%). The incidence of carriers in patients without cancer but a known family history of breast cancer (8.2%), on the other hand, was slightly higher than cancer-free volunteers but lower than breast or colon cancer patients. Our results indicate a possible hereditary trend for both breast cancer and colon cancer; however, a much larger population is needed to definitively determine the potential contribution of this locus to risk for hereditary cancers. The incidence of this potential biomarker should also be examined in other potentially heritable cancers, such as ovarian cancer, which is known to be linked to familial (especially BRCA1/2-associated) breast cancer³⁵.

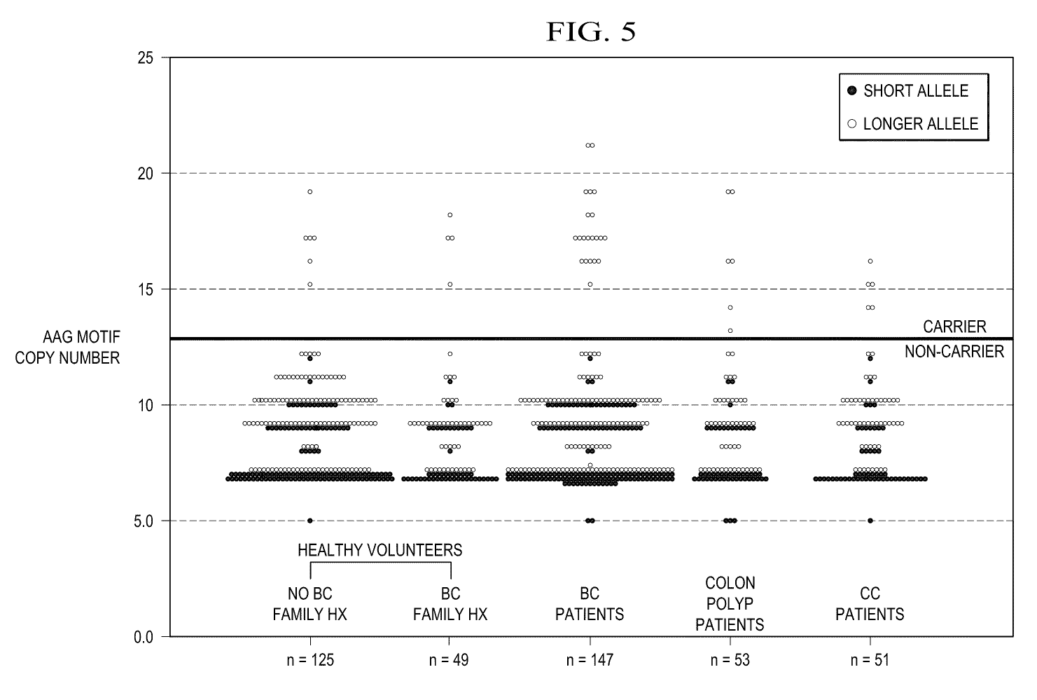

The distribution of the allele sizes for the different patient groups is shown in FIG. 5. The reference genome contains 8 copies; although this motif was relatively rare among the patient samples we tested (only 48 alleles were found with 8 copies of the motif, compared to 369, 181 and 119 alleles that had 7, 9 and 10 copies, respectively). Observed allelic frequencies of long (n=13+ copies) and short alleles is consistent with Hardy-Weinberg equilibrium. No correlation related to gender (the majority of samples, ˜80%, were female) or race/ethnicity was apparent (Table 6), although a much larger patient population would be required to confirm this.

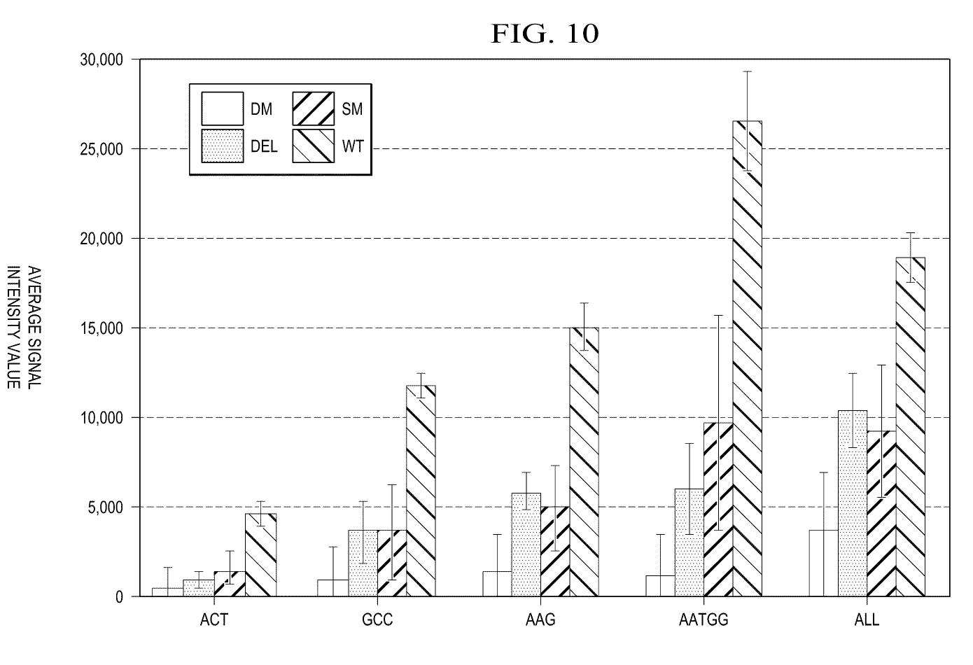

FIG. 10 shows the results of an analysis of control probes indicates that the global microsatellite content array confirms binding specificity. Comparison of normalized signal values for probes representing wild-type (WT), single mismatch (SM), double mismatch (DM), and deletion (Del) probes for four representative microsatellite motifs and also the average of all motifs on the array was used as a measure of array specificity. The average signal intensities shown were calculated based on all cyclic permutations for the given motif for all 53 DNA samples hybridized to the array. The resulting averages are displayed on the ordinates, and the standard deviations are shown as error bars. Note that specificity decreases as alterations are made to the center nucleotide base, and standard deviations are lowest for perfect match (WT) probes. Comparisons were made for all microsatellite motifs represented on the array, and the four motifs shown were chosen to represent a broad range of intensity values. Note that all WT motif signals exceeded their corresponding mismatch probes, confirming binding specificity

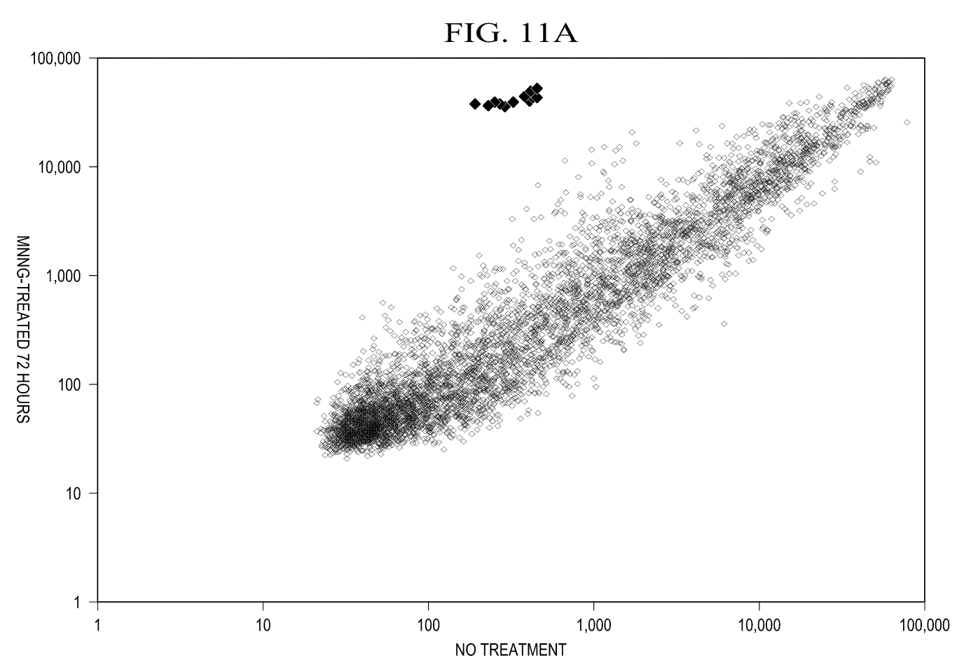

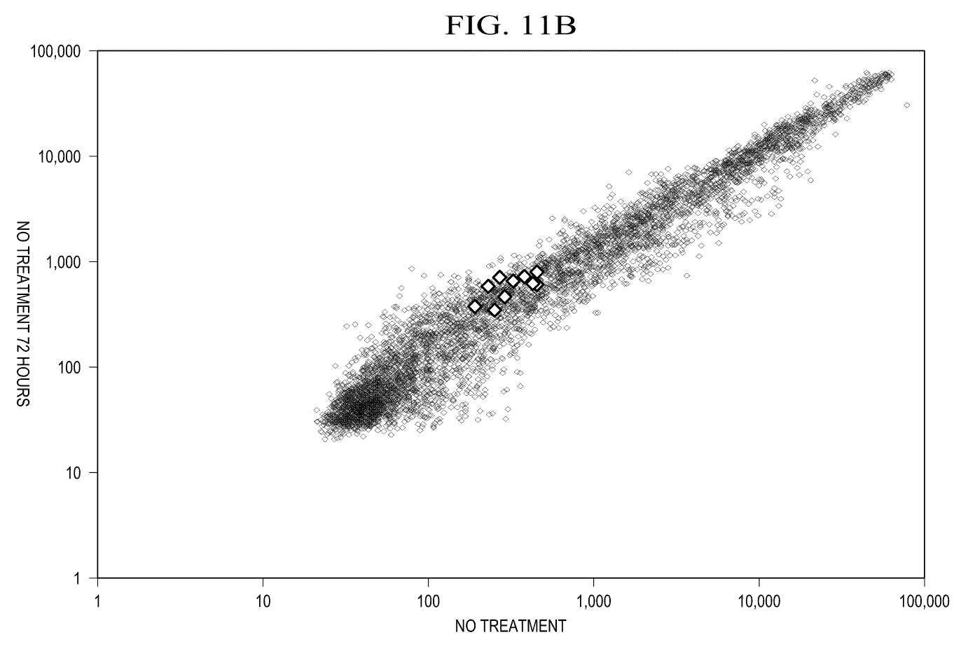

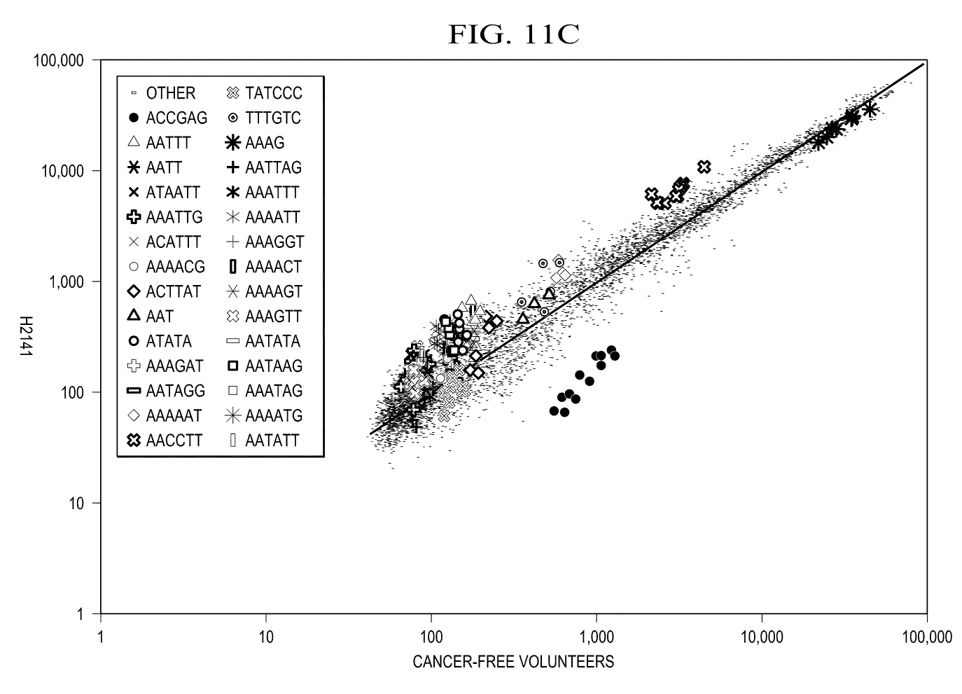

Colon cells exposed to MNNG (alkylating agent) for 72 hours and specific DNA damage after treatment with alkylating agents over time (FIGS. 11A and 11B). FIG. 11C shows the comparison of Lung cancer patient DNA to DNA from cancer-free volunteers. Distinct, reliable and reproducible patterns of DNA changes are detected within a single species, in this case, humans. Similar patterns measured for breast, colon, and childhood cancers, thus creating a universal signature for cancer.

Microsatellites are mainly understudied despite their known connection with cancer and other diseases (e.g., neurological developmental defects), because there has never been a method for assaying them en masse until now. In this study, we describe a new method for the detection and comparison of global microsatellite changes, a technique that is both sensitive and specific. There are multiple potential applications for this new array, which can detect a single contaminating microsatellite motif, present at a calculated concentration as low as 2-5 copies per cell³⁶⁻³⁸, as was demonstrated with EBV-transformed B lymphocyte DNA (FIG. 1).

We found a set of commonly destabilized repetitive microsatellite motifs in tumors and germ lines, a pattern that may represent a cancer predisposition biomarker. Notably, whereas the pattern of microsatellite expansion was seen in the germ lines as well as the tumors in breast and colon cancer patients, the pattern was seen only in the tumor line derived from a small cell lung carcinoma patient. It is possible that this difference may be related to the relative importance of environmental factors versus genetic predisposition in the etiology of these different neoplasms. We might expect that lung cancer, because it is usually caused by tobacco exposure, would be less likely to be associated with underlying genetic risk factors.

Most of the microsatellites altered in cancer patients consist of multiples of nucleotides A and T; that is, the differential motif sequence usually takes the form of AₙTₘ. Further research will be needed to ascertain the reason for this pattern, but the fact that particular repeat motifs are mutated more commonly suggests that there is sequence bias in the DNA repair machinery in tumors favoring errors in such motifs. It is also interesting to note that the distribution of microsatellites found to be variable between cancer-free volunteers and cancer patients strongly favors microsatellites that are located outside gene coding regions. Indeed, only one of the 42,702 loci that contain these microsatellites lies within an exon (Table 4), suggesting that there is extreme selection pressure against these particular motifs within coding regions. There are 1,124 1- to 6-mer microsatellites located in exons out of ˜507,000 computationally identified in the human reference genome, which equals ˜0.2%. So, the expected value in the set of microsatellites identified as differential should be 95, much higher than what was actually observed (i.e., only 1).

Differential motifs discovered using this array can lead to the discovery of specific disease-associated genetic loci. For example, after measuring the increased hybridization signal reflecting alterations in tandem repeats of the AAAG motif, we were able to consider which of the genes near these microsatellites might be expected to affect cancer behavior and then subject these loci to more detailed analysis. We discovered a variable repetitive motif in the 5′ UTR of ERR-γ that exhibits a significantly higher incidence in patients with breast cancer and possibly colon neoplasia. ERR-γ expression has previously been implicated as a potential prognostic marker in breast cancer^{33,39}. ERR-γ has 2 known isoforms, one with an alternative first exon and one with an alternative 5′ UTR. It is possible that the differential AAAG microsatellite confers alternate regulation of ERR-γ, as is thought to be the case for the gene encoding the parathyroid hormone receptor, which also harbors a polymorphic (AAAG)ₙ repeat sequence in its promoter region that co-varies with adult height³⁴. There are 22 candidate transcription factors (see FIGS. 7A-7F) that could potentially bind to the region of the 5′UTR of ERR-γ containing the AAAG repeat (the repeat itself plus 100 by flanking sequences), one of which (paired box gene 2, PAX2) is capable of binding the repeat unit itself. This finding suggests a potential mechanism of action, as PAX2 was recently implicated in estrogen receptor (ER)-mediated regulation of ERBB2 (v-erb-b2 erythroblastic leukemia viral oncogene homolog 2) and resistance to the breast cancer treatment agent, tamoxifen⁴⁰, and ERRSG has been shown to mediate tamoxifen-resistance in a cell model that represents invasive lobular breast carcinoma³³. Further studies would be required to determine if PAX2 or other transcription factor binding sites in close proximity to the repeat (shown in FIGS. 7A-7F) are affected by (AAAG)ₙ length variations.

Because microsatellites have in many cases been shown to impact expression of adjacent genes^{14,41}, it is interesting to speculate that ERR-γ expression differences related to the different AAAG copy number may impact breast cancer risk. If the frequency of this potentially predictive marker is sustained in a larger population, and the mechanism by which it confers the cancer phenotype can be identified, it may contribute substantially as a biomarker offering surveillance, prophylactic surgery, and chemoprevention options to patients. Based on our assessment, this allele carries a 2.97 relative risk. As a comparison, deleterious germ line mutations of the BRCA1 gene have a 3-7% frequency in breast cancer patients (age <45), which is significantly elevated in those with a family history (up to 33%). Such mutations are associated with a 3-7 times higher risk of breast cancer, compared to non-mutation carriers^{42,43}. The incidence of BRCA1 mutation in the general population is estimated at 0.2 to 0.4%⁴⁴.

The potential role of microsatellites in a number of different neoplasms as demonstrated in this work is significantly greater than might be predicted given the individual locus discoveries to date. Whereas microsatellite instability has been sporadically demonstrated in a large number of tumors, consistent MSI has been seen most commonly in colorectal carcinoma and endometrial carcinoma. It should be noted that the standard assay for MSI compares microsatellite length for an extremely limited set of loci between tumor DNA and non-tumor DNA from the same patient. Because we have found alterations in microsatellite differences that affect germ line DNA, they would not be detected by the standard MSI assay. Indeed, what we have described (in the case of breast, cancer and hepatoblastoma tumors) would not be regarded as MSI, since the microsatellite patterns do not differ in the tumor from the normal tissue. However, we have found that assaying more widely for alterations in microsatellite content reveals abnormalities in other tumor types as well. Based on our results, global microsatellite content may be used to distinguish individuals at higher risk of developing cancer and may be a better gauge of “MSI”.

It is provocative to consider the similarities and differences between the microsatellite patterns observed in DNA derived from tumor tissue when compared to the DNA obtained from normal tissue. Primary breast cancer tumors exhibit significantly increased hybridization of some microsatellite motifs, a pattern also seen in non-tumor DNA from these patients, when compared to the DNA obtained from a set of cancer-free individuals. A similar concurrence of microsatellites is seen in the embryonal tumor hepatoblastoma. That these altered microsatellite patterns are found in DNA from both tumor and germ line DNA suggests that such alterations may predispose to the development of cancer. This pattern contrasts with the pattern seen in lung cancer; whereas the tumor exhibits an altered microsatellite pattern, the germ line is not different from cancer-free subjects. Thus, in lung cancer patients, the carcinogenic insult may induce the development of microsatellite alterations that contribute to neoplastic transformation. These results further suggest that these microsatellite motifs in particular are a clue to the underlying mechanism responsible, which may be a target to intercept the oncogenesis process. Interestingly, we found microsatellite alterations in colon cancer tumors, in which there was variable presence of this genotype in the germ line. Perhaps colon cancer resides in the middle of the scale measuring the relative importance of the underlying genetic milieu versus the importance of environmental factors in the development of malignancy, which is consistent with the highly variable exposure of the colon to different foods.

A larger scale study may be merited to determine if global microsatellite content signatures can also be used as a reliable biomarker for tumor sub-type classification and prediction of prognosis or response to therapy. The abnormal microsatellite signatures potentially implicate thousands of genetic loci. Investigation of a very small subset led to significant findings. This suggests that there may be many more important repeat-containing loci affecting cancer development or progression that are yet to be identified.

Hepatitis C virus: 6 of 12 genomes downloaded contained a 20 bp “T” repeat. Human T-lymphotropic virus: No 18 to 20 bp microsats found. 6 out of 16 genomes downloaded contained a 12 bp CCAGAG microsat. Human herpes virus 8: 2 out of 3 genomes contained a 20 bp “G” repeat. All 3 had a CCTGCT repeat. Lengths were (2) 23 bps and (1) 17 bps.

TABLE 2
Genomes Hybridized to the Array

| | | | |
|---|---|---|---|
| Sample ID | Sex | Tissue | Description |

Primary Tissue and Blood Samples

| | | | |
|---|---|---|---|
| N1 | M | Blood | Cancer-free male volunteer (Caucasian) |
| N2 | M | Blood | Cancer-free male volunteer (East Indian) |
| N3 | M | Blood | Cancer-free male volunteer (Chinese) |
| N4 | F | Blood | Cancer-free female volunteer (Mixed race) |
| N5 | F | Blood | Cancer-free female volunteer (Caucasian) |
| N6 | F | Blood | Cancer-free female volunteer (Caucasian) |
| N1-EBVt | M | Blood | H1 EBV-transformed cells |
| N4-EBVt | F | Blood | H5 EBV-transformed cells |
| BC(1-5)T | F | Breast | Basal-type breast cancer patient tissue |
| BC(1-5)G | F | Blood | Matching breast cancer patient blood |
| BC(6-10)T | F | Breast | Luminal-type breast cancer patient tissue |
| BC(6-10)G | F | Blood | Matching breast cancer patient blood |
| H(1-3)T | — | Liver | Childhood hepatoblastoma tumor tissue (non-syndromic): |
| | | | childhood liver cancer at very young age of onset suggestive of |
| | | | genetic predisposition |
| H(1-3)G | — | Blood | Matching childhood hepatoblastoma patient blood |
| CC1T | — | Colon | Colon cancer patient tissue |
| CC1G | — | Blood | Matching blood sample |
| CC2T | — | Colon | Colonic adenocarcinoma w/signet ring features, Grade III, |
| | | | Stage T4N2M1 |
| CC2G | — | Small | Benign perilesional tissue |
| | | intestine | |
| CC3T | — | Colon | Invasive adenocarcinoma, Grade II, Stage T3N1M1 |
| CC3G | — | Liver | Benign liver (exploratory laparotomy) - cancer later |
| | | | metastasized to liver, patient deceased |

Established Cancer and B Lymphocyte Cell Lines

| | | | |
|---|---|---|---|
| RKO | — | Colorectal | Poorly differentiated colorectal carcinoma cell line |
| HCT15 | M | Colorectal | Duke's Type C colorectal adenocarcinoma |
| HCT116 | M | Colorectal | Colorectal carcinoma |
| HCC1187 | F | Breast | TNM Stage IIA, grade 3 primary ductal carcinoma |
| HCC1187BL | F | Blood | Matched blood cell line |
| HCC1395 | F | Breast | TNM Stage I, grade 3 primary ductal carcinoma |
| HCC1395BL | F | Blood | Matched blood cell line |
| HCC2157 | F | Breast | TNM Stage IIIA, grade 2 primary ductal carcinoma |
| HCC2157BL | F | Blood | Matched blood cell line |
| H1437 | M | Lung | Stage 1 adenocarcinoma, non-small cell lung cancer; patient |
| | | | was smoker (70 pack years) |
| BL1437 | M | Blood | Matched blood cell line |
| H2141 | M | Lung | Stage E carcinoma, small cell lung cancer; patient was smoker |
| | | | (50 pack years) |
| BL2141 | M | Blood | Matched blood cell line |
| H2887 | M | Lung | — |
| BL2887 | M | Blood | Matched blood cell line |

Notes:
A dash (“—”) indicates that the information was not available. All cell lines and volunteer blood samples were also included in a small PCR panel of 42 samples used to test individual loci (discussed below).

TABLE 3
Application of standard MSI testing kit

| Bethesda Markers | | | | | | |
|---|---|---|---|---|---|---|
| | | | | MONO- | Control Markers | |
| NR-21 | BAT-26 | BAT-25 | NR-24 | 27 | Penta C | Penta D |

| Normal range | 94-101 | 103-115 | 114-124 | 130-133 | 148-154* | 143-194 | 135-201 |
|---|---|---|---|---|---|---|---|
| Samples | Allele 1/Allele 2 (bp) | | | | | | |
| Control | 101/101 | 113/113 | 122/122 | 130/130 | 149/149 | 164/174 | 168/187 |
| N1 | 99/99 | 113/113 | 122/122 | 131/131 | 150/150 | 174/179 | 168/168 |
| N2 | 98/98 | 113/113 | 122/122 | 131/131 | 150/150 | 169/169 | 177/181 |
| N3 | 99/99 | 115/115 | 122/122 | 130/130 | 150/150 | 164/164 | 168/177 |
| N4 | 98/98 | 113/113 | 121/121 | 130/130 | 150/150 | 164/174 | 135/181 |
| N5 | 99/99 | 113/113 | 122/122 | 130/130 | 149/149 | 174/194 | 177/181 |
| N6 | 99/99 | 113/113 | 121/121 | 131/131 | 149/149 | 159/164 | 177/181 |
| N7 | 99/99 | 113/113 | 122/122 | 131/131 | 150/150 | 174/179 | 168/181 |
| N8 | 99/99 | 113/113 | 122/122 | 130/130 | 150/150 | 179/184 | 168/168 |
| N9 | 99/99 | 113/113 | 123/123 | 131/131 | 150/150 | 164/174 | 162/168 |
| N10 | 97/97 | 113/113 | 122/122 | 130/130 | 149/149 | 164/179 | 147/181 |
| N11 | 98/98 | 113/113 | 122/122 | 131/131 | 150/150 | 164/184 | 172/187 |
| N12 | 99/99 | 113/113 | 123/123 | 130/130 | 150/150 | 164/174 | 168/181 |
| N13 | 99/99 | 113/113 | 122/122 | 131/131 | 151/151 | 174/179 | 168/172 |
| N14 | 98/98 | 113/113 | 121/121 | 130/130 | 150/150 | 174/184 | 135/139 |
| N15 | 98/98 | 113/113 | 121/121 | 130/130 | 150/150 | 174/184 | 177/191 |
| N16 | 98/98 | 113/113 | 122/122 | 131/131 | 150/150 | 164/174 | 181/181 |
| N17 | 98/98 | 113/113 | 122/122 | 130/130 | 149/149 | 164/184 | 168/177 |
| H2141 | 99/99 | 113/113 | 122/122 | 131/131 | 150/150 | 179/184 | 172/177 |
| BL2141 | 99/99 | 113/113 | 122/122 | 131/131 | 150/150 | 179/184 | 172/177 |
| H1437 | 99/99 | 113/113 | 122/122 | 131/131 | 150/150 | 179/184 | 172/181 |
| BL1437 | 99/99 | 113/113 | 122/122 | 131/131 | 150/150 | 179/184 | 172/181 |
| H2887 | 98/98 | 113/113 | 122/122 | 130/130 | 149/149 | 174/174 | 181/181 |
| BL2887 | 98/98 | 113/113 | 122/122 | 130/130 | 149/149 | 174/179 | 181/181 |
| HCC1007 | 97/97 | 113/113 | 121/121 | 130/130 | 150/150 | 179/179 | 162/181 |
| HCC1007BL | 97/97 | 113/113 | 121/121 | 130/130 | 150/150 | 164/179 | 162/181 |
| HCC1187 | 99/99 | 113/113 | 122/122 | 131/131 | 150/150 | 174/174 | 177/177 |
| HCC1187BL | 99/99 | 113/113 | 122/122 | 131/131 | 150/150 | 174/174 | 172/177 |
| HCC2157 | 99/99 | 113/113 | 121/121 | 130/130 | 150/150 | 164/179 | 162/172 |
| HCC2157BL | 98/98 | 113/113 | 122/122 | 130/130 | 150/150 | 164/179 | 162/172 |
| HCC1395 | 99/99 | 113/113 | 122/122 | 130/130 | 150/150 | 174/174 | 181/181 |
| HCC1395BL | 99/99 | 113/113 | 122/122 | 130/130 | 150/150 | 174/174 | 181/181 |
| CC1T | 99/99 | 113/113 | 121/121 | 130/130 | 150/150 | 159/174 | 162/177 |
| CC1G | 99/99 | 113/113 | 121/121 | 130/130 | 150/150 | 159/174 | 162/177 |
| CC2T | 98/98 | 115/115 | 121/121 | 131/131 | 150/150 | 174/179 | 168/168 |
| CC2G | 98/98 | 113/113 | 121/121 | 131/131 | 150/150 | 179/184 | 177/177 |
| CC3T | 98/98 | 113/113 | 121/121 | 131/131 | 150/150 | 179/184 | 177/177 |
| CC3G | 98/98 | 115/115 | 122/122 | 131/131 | 150/150 | 174/179 | 168/168 |
| BC1T | 98/98 | 113/113 | 121/121 | 130/130 | 150/150 | 179/179 | 181/187 |
| BC2T | 99/99 | 113/113 | 123/123 | 131/131 | 150/150 | 179/184 | 172/187 |
| BC3T | 99/99 | 113/113 | 121/121 | 130/130 | 150/150 | 174/174 | 187/187 |
| BC6T | 98/98 | 113/113 | 121/121 | 130/130 | 150/150 | 174/179 | 187/187 |
| BC7T | 99/99 | 113/113 | 121/121 | 131/131 | 150/150 | 174/174 | 172/181 |
| HCT15 | 96/96 | 109/109 | 113/119 | 127/127 | 146/146 | 169/174 | 168/191 |
| HCT116 | 92/92 | 102/102 | 116/116 | 120/126 | 142/142 | 164/169 | 168/187 |
| RKO | 86/89 | 101/101 | 112/112 | 121/124 | 136/136 | 174/174 | 172/177 |

*The frequency of this range was 99.8% (out of 538 people tested by Suraweera et al., 2002) - only 1 person tested outside of this range (Promega technical document MD1641). Values outside of the normal range are highlighted in red. Cancer-free volunteer samples are labeled as N1-17, and cell lines are labeled in accordance with accepted nomenclature. Colon cancer patient samples are labeled CC1T-3T for cancerous tissues and CC1G-3G for germ lines (matching B lymphocytes or benign tissue). Basal-type breast cancer samples are labeled as BC1T-3T, and luminal-type breast cancer samples are designated as BC6T and 7T.
Suraweera, N. et al. (2002) Evaluation of tumor microsatellite instability using five quasimonomorphic mononucleotide repeats and pentaplex PCR. Gastroenterology 123, 1804-11.

TABLE 4
Genomic locations of microsatellites found to be globally differential
between cancer patients and cancer-free volunteers

| | Up | Down | 5′ | 3′ | | | | |
|---|---|---|---|---|---|---|---|---|
| Motif | stream | Stream | UTR | UTR | Intron | Exon | Intergenic | Total |
| AAAGAC | 1 | 0 | 1 | 0 | 11 | 1 | 24 | 38 |
| AATTT | 2 | 2 | 35 | 6 | 193 | 0 | 452 | 690 |
| AATT | 2 | 5 | 42 | 7 | 277 | 0 | 553 | 886 |
| AATTAG | 0 | 0 | 1 | 0 | 7 | 0 | 27 | 35 |
| ATAATT | 0 | 0 | 0 | 0 | 21 | 0 | 75 | 96 |
| AAATTT | 0 | 0 | 15 | 1 | 90 | 0 | 150 | 256 |
| AAATTG | 0 | 0 | 0 | 0 | 9 | 0 | 24 | 33 |
| AAAATT | 3 | 2 | 38 | 8 | 246 | 0 | 462 | 759 |
| ACATTT | 0 | 1 | 2 | 1 | 12 | 0 | 39 | 55 |
| AAAACG^{†} | 0 | 0 | 0 | 0 | 0 | 0 | 0 | 0 |
| AAAACT | 0 | 1 | 3 | 0 | 22 | 0 | 34 | 60 |
| ACTTAC | 0 | 0 | 0 | 0 | 0 | 0 | 2 | 2 |
| AAAAAT | 63 | 79 | 496 | 85 | 3,173 | 0 | 5,639 | 9,535 |
| AAAAGT | 0 | 0 | 2 | 0 | 8 | 0 | 17 | 27 |
| AAT | 74 | 67 | 732 | 134 | 4,588 | 0 | 8,865 | 14,460 |
| AAAGTT | 0 | 0 | 0 | 0 | 1 | 0 | 8 | 9 |
| ATATA | 3 | 1 | 11 | 2 | 99 | 0 | 363 | 479 |
| AAATAT | 1 | 1 | 17 | 6 | 154 | 0 | 383 | 562 |
| AAAGAT | 0 | 0 | 1 | 0 | 7 | 0 | 10 | 18 |
| AATAAG | 1 | 0 | 1 | 0 | 18 | 0 | 39 | 59 |
| AATAGG | 1 | 0 | 0 | 1 | 3 | 0 | 6 | 11 |
| AAATAG | 0 | 0 | 2 | 0 | 18 | 0 | 50 | 70 |
| AAAATG | 0 | 0 | 8 | 1 | 23 | 0 | 49 | 81 |
| AACCTT | 1 | 0 | 0 | 1 | 1 | 0 | 7 | 10 |
| AATATT | 0 | 0 | 6 | 1 | 32 | 0 | 103 | 142 |
| AAAGGT | 0 | 0 | 0 | 1 | 1 | 0 | 5 | 7 |
| AAAG^{‡} | 102 | 53 | 608 | 112 | 3,252 | 0 | 10,184 | 14,311 |

Only genes in the RefSeq database were included. A “count” is defined as a complete tandem repeat at least 18 bp (for 3-mers and 6-mers) or 20 bp (for 1-, 2-, 4-, 5-, and 6-mers), in length. Upstream and downstream were defined as 1,000 bp distal from the transcribed gene. ^{†}No copies of this motif were found using 18 bp as the threshold, but at 12 bp there
were 438 copies detected in the human reference genome assembly. ^{‡}This motif was highly statistically significant for all cancers tested (B-H adjusted p value ~0.0003), but it was not included in the canonical set of motifs shown in FIG. 4 due to failure to meet a magnitude difference threshold (only ~35% difference in signal intensity between cancer-free
volunteers and cancer patient samples).

TABLE 5
Genotyping results various samples (patients, volunteers, and cell lines) for the AAAG motif in the 5′
UTR of ERR-γ

| | | | | | | | |
|---|---|---|---|---|---|---|---|
| Sex | Age | Ethnicity | BRCA ½ | Disease status | Family hx of cancer | Allele 1 | Allele 2 |

Healthy volunteers - no BC family history

| | | | | | | | |
|---|---|---|---|---|---|---|---|
| F | N/K | Mixed Ethnicity | N/K | No cancer | No | 10 | 11 |
| F | N/K | Chinese | N/K | No cancer | No | 12 | 12 |
| F | 40 | African American | N/K | No cancer | No | 7 | 7 |
| F | 41 | White | N/K | No cancer | No | 7 | 10 |
| F | 32 | Hispanic | N/K | No cancer | No | 9 | 11 |
| F | 45 | Hispanic | N/K | No cancer | No | 7 | 9 |
| F | 64 | Caucasian | N/K | No cancer | No | 10 | 10 |
| F | 55 | Hispanic | N/K | No cancer | No | 7 | 10 |
| F | 40 | Caucasian | N/K | No cancer | No | 7 | 9 |
| F | 37 | N/K | N/K | No cancer | No | 7 | 9 |
| F | 53 | Caucasian | N/K | No cancer | No | 9 | 11 |
| F | 27 | Hispanic | N/K | No cancer | No | 7 | 10 |
| F | 38 | African American | N/K | No cancer | No | 7 | 9 |
| F | 39 | Caucasian | N/K | No cancer | No | 7 | 9 |
| F | 61 | N/K | N/K | No cancer | No | 7 | 9 |
| F | 38 | Native | N/K | No cancer | No | 10 | 11 |
| | | American/White | | | | | |
| F | 70 | Caucasian | N/K | No cancer | No | 7 | 10 |
| F | 44 | Caucasian | N/K | No cancer | No | 8 | 10 |
| F | 25 | Caucasian | N/K | No cancer | No | 9 | 11 |
| F | N/K | White | N/K | No cancer | No | 7 | 10 |
| F | 32 | Caucasian | N/K | No cancer | No | 10 | 10 |
| F | 50 | Caucasian | N/K | GERD | No | 10 | 10 |
| F | 48 | Caucasian | N/K | GERD | No | 7 | 7 |
| M | 65 | Caucasian | N/K | No cancer | No | 9 | 10 |
| M | 71 | N/K | N/K | No cancer | No | 7 | 7 |
| M | 57 | N/K | N/K | No cancer | No | 7 | 7 |
| M | N/K | Caucasian | N/K | No cancer | No | 7 | 7 |
| M | 62 | Caucasian | N/K | No cancer | No | 7 | 9 |
| M | 55 | N/K | N/K | No cancer | No | 9 | 11 |
| M | N/K | White | N/K | No cancer | No | 7 | 9 |
| M | N/K | Asian/Chinese | N/K | No cancer | No | 7 | 7 |
| M | N/K | White | N/K | No cancer | No | 9 | 10 |
| M | N/K | Asian/Indian | N/K | No cancer | No | 7 | 7 |
| M | N/K | African | N/K | No cancer | No | 7 | 7 |
| M | 23 | Caucasian | N/K | No cancer | No | 7 | 9 |
| M | 59 | Caucasian | N/K | No cancer | No | 7 | 9 |
| M | 24 | Chinese | N/K | No cancer | No | 7 | 10 |
| M | 22 | Asian Indian | N/K | No cancer | No | 9 | 9 |
| F | 23 | Asian Indian | N/K | No cancer | No | 7 | 11 |
| F | 23 | White-Hispanic | N/K | No cancer | No | 9 | 10 |
| M | 33 | Chinese | N/K | No cancer | No | 7 | 7 |
| F | 30 | Caucasian | N/K | No cancer | No | 7 | 7 |
| F | 42 | Caucasian | N/K | No cancer | No | 10 | 17 |
| F | 36 | Caucasian | Neg | No breast cancer | No | 8 | 11 |
| F | 48 | Caucasian | N/K | No cancer | No | 9 | 9 |
| F | 35 | Black | N/K | No cancer | No | 8 | 8 |
| F | 50 | Hispanic | N/K | No cancer | No | 7 | 12 |
| F | 58 | Caucasian | N/K | No cancer | No | 7 | 7 |
| F | 51 | Caucasian | N/K | No cancer | No | 9 | 17 |
| N/K | 58 | Caucasian | N/K | No cancer | No | 7 | 9 |
| F | 49 | Caucasian | N/K | No cancer | No | 9 | 11 |
| N/K | 55 | Asian | N/K | No cancer | No | 7 | 10 |
| | 49 | Asian | N/K | No cancer | No | 7 | 9 |
| F | 73 | Hispanic | N/K | No cancer | No | 7 | 7 |
| F | 57 | Caucasian | N/K | No cancer | No | 7 | 10 |
| N/K | 59 | Asian | N/K | No cancer | No | 7 | 7 |
| F | 64 | Caucasian | N/K | No cancer | No | 7 | 9 |
| M | 35 | Asian | N/K | No cancer | No | 7 | 7 |
| F | 65 | N/K | N/K | Cysts of uterus | No | 9 | 10 |
| | | | | and fallopian tube | | | |
| F | 64 | N/K | N/K | Cystic ovaries | No | 8 | 9 |
| F | 34 | Caucasian | N/K | Ovarian cyst | No | 7 | 9 |
| F | 37 | Hispanic | N/K | Endometriotic | No | 7 | 9 |
| | | | | cyst | | | |
| F | 40 | Hispanic | N/K | Ovarian cyst | No | 7 | 11 |
| F | 49 | Hispanic | N/K | Ovarian cyst | No | 7 | 7 |
| F | 66 | Caucasian | N/K | Ovarian cyst | No | 7 | 11 |
| F | 54 | Caucasian | N/K | Fibroma | No | 9 | 9 |
| F | 41 | N/K | N/K | Endometrial cyst | No | 9 | 15 |
| F | 44 | Hispanic | N/K | Ovarian cyst | No | 9 | 11 |
| F | 54 | African American | N/K | Ovarian cyst | No | 7 | 8 |
| F | 65 | Caucasian | N/K | Ovarian cyst | No | 9 | 9 |
| F | 60 | African American | N/K | Ovarian cyst | No | 7 | 8 |
| F | 62 | African American | N/K | Ovarian cyst | No | 7 | 7 |
| F | 40 | Caucasian | N/K | Benign phyllodes | No | 7 | 11 |
| | | | | tumor | | | |
| F | 42 | African American | N/K | Breast | No | 7 | 7 |
| | | | | Fibroadenoma | | | |
| F | 32 | African American | N/K | Ovarian cyst | No | 7 | 8 |
| F | 39 | Caucasian | N/K | Fibrocystic | No | 9 | 11 |
| | | | | breasts | | | |
| F | 47 | Indian | N/K | Ovarian cyst | No | 7 | 7 |
| F | 60 | Caucasian | N/K | No cancer | No | 7 | 10 |
| F | 36 | N/K | N/K | No cancer | No | 7 | 7 |
| F | 44 | N/K | N/K | No cancer | No | 7 | 7 |
| F | 49 | Hispanic | N/K | No cancer | No | 7 | 10 |
| F | 58 | Caucasian | N/K | No cancer | No | 10 | 10 |
| F | 57 | Caucasian | N/K | No cancer | No | 7 | 10 |
| F | 43 | Caucasian | N/K | No cancer | No | 7 | 12 |
| F | 55 | Hispanic | N/K | No cancer | No | 11 | 12 |
| F | 41 | African American | N/K | No cancer | No | 7 | 7 |
| F | 55 | Caucasian | N/K | No cancer | No | 7 | 9 |
| F | 49 | Hispanic | N/K | No cancer | No | 9 | 19 |
| F | 60 | Caucasian | N/K | No cancer | No | 7 | 17 |
| F | 55 | Caucasian | N/K | No cancer | No | 7 | 7 |
| F | 82 | Caucasian | N/K | No cancer | No | 7 | 9 |
| F | 61 | Hispanic | N/K | No cancer | No | 7 | 9 |
| F | 73 | Caucasian | N/K | No cancer | No | 7 | 10 |
| F | 61 | African American | N/K | Endometrial | No | 7 | 9 |
| | | | | hyperplasia & | | | |
| | | | | polyps | | | |
| F | N/K | N/K | N/K | No cancer | N/K | 9 | 11 |
| F | N/K | N/K | N/K | No cancer | N/K | 5 | 7 |
| F | 58 | Black | N/K | No cancer | N/K | 9 | 10 |
| | | N01-01-001 | | No cancer | | 7 | 8 |
| | | N01-01-002 | | No cancer | | 7 | 7 |
| | | N01-01-004 | | No cancer | | 7 | 9 |
| | | N01-01-003 | | No cancer | | 9 | 10 |
| | | N01-01-006 | | No cancer | | 7 | 7 |
| | | N01-01-015 | | No cancer | | 7 | 9 |
| | | N01-01-017 | | No cancer | | 7 | 7 |
| | | N01-01-021 | | No cancer | | 10 | 10 |
| | | N01-01-022 | | No cancer | | 10 | 16 |
| | | N01-01-024 | | No cancer | | 8 | 11 |
| | | N01-01-026 | | No cancer | | 7 | 7 |
| | | N01-01-027 | | No cancer | | 7 | 10 |
| | | N01-01-029 | | No cancer | | 7 | 10 |
| | | N01-01-030 | | No cancer | | 7 | 10 |
| | | N01-01-031 | | No cancer | | 7 | 9 |
| | | N01-01-032 | | No cancer | | 10 | 10 |
| | | N01-01-035 | | No cancer | | 9 | 9 |
| | | N01-01-037 | | No cancer | | 9 | 9 |
| | | N01-01-040 | | No cancer | | 10 | 12 |
| | | N01-01-045 | | No cancer | | 9 | 10 |
| | | N01-01-047 | | No cancer | | 9 | 10 |
| | | N01-01-049 | | No cancer | | 7 | 7 |
| | | N01-01-052 | | No cancer | | 7 | 9 |
| | | N01-01-053 | | No cancer | | 7 | 7 |
| | | N01-01-054 | | No cancer | | 7 | 9 |
| | | N01-01-055 | | No cancer | | 7 | 9 |
| | | N01-01-056 | | No cancer | | 10 | 11 |
| | | N01-01-059 | | No cancer | | | |

Healthy volunteers - family hx of breast cancer

| | | | | | | | |
|---|---|---|---|---|---|---|---|
| F | 37 | African | Neg | No cancer | Maternal aunt, mother, | 11 | 17 |
| | | American | | | maternal grandmother, | | |
| | | | | | maternal cousin with | | |
| | | | | | breast cancer | | |
| F | 29 | Caucasian | Neg | No cancer | Maternal cousin, | 7 | 9 |
| | | | | | maternal aunt with | | |
| | | | | | breast cancer | | |
| F | 45 | Asian | BRCA1− | Fibrocystic breast | Maternal cousin, | 9 | 9 |
| | | | | disease | maternal aunt, sister | | |
| | | | | | with breast cancer | | |
| F | 43 | African American | BRCA1− | No cancer | Maternal cousin, | 7 | 7 |
| | | | | | maternal aunt, sister | | |
| | | | | | with breast cancer | | |
| F | 53 | Caucasian | Neg | No cancer | Maternal cousin, sister, | 7 | 7 |
| | | | | | mother with breast | | |
| | | | | | cancer | | |
| F | 45 | Caucasian | Neg | No cancer | Maternal grandmother, | 9 | 9 |
| | | | | | maternal aunt, mother | | |
| | | | | | with breast cancer | | |
| F | 36 | Caucasian | Neg | No cancer | Maternal grandmother, | 7 | 9 |
| | | | | | mother with breast | | |
| | | | | | cancer | | |
| F | 34 | N/K | BRCA2+ | No cancer | Maternal great aunt, | 7 | 7 |
| | | | | | maternal aunt, and | | |
| | | | | | mother with breast | | |
| | | | | | cancer | | |
| F | 21 | Caucasian | BRCA2+ | No cancer | Maternal great | 7 | 9 |
| | | | | | grandmother, maternal | | |
| | | | | | great aunt, mother with | | |
| | | | | | breast cancer | | |
| F | 44 | African American | Neg | No cancer | Maternal great uncle, | 7 | 8 |
| | | | | | maternal aunt, maternal | | |
| | | | | | grandmother, and | | |
| | | | | | mother with breast | | |
| | | | | | cancer | | |
| F | 35 | Native American | Neg | Fibrodenoma with | Mother with breast | 7 | 7 |
| | | | | myxoid stroma | cancer | | |
| F | 36 | Caucasian | BRCA2− | No cancer | Mother and maternal | 9 | 17 |
| | | | | | aunt with breast | | |
| | | | | | cancer | | |
| F | 70 | Caucasian | Neg | No cancer | Mother and two niece | 9 | 15 |
| | | | | | with breast cancer | | |
| F | 43 | African American | Neg | benign | Mother with breast | 7 | 8 |
| | | | | hemorrhagic | cancer | | |
| | | | | follicular cyst | | | |
| F | 38 | Caucasian | Neg | No cancer | Mother with breast | 7 | 7 |
| | | | | | cancer | | |
| F | 36 | Caucasian | Neg | No cancer | Mother with breast | 7 | 7 |
| | | | | | cancer | | |
| F | 31 | Caucasian | Neg | No cancer | Mother with breast | 8 | 9 |
| | | | | | cancer | | |
| F | 46 | Caucasian | Neg | No cancer | Mother with breast | 10 | 11 |
| | | | | | cancer | | |
| F | 37 | Caucasian | Neg | Fibroadenoma | Mother with breast | 9 | 9 |
| | | | | | cancer; paternal aunt | | |
| | | | | | with ovarian cancer | | |
| F | 42 | Hispanic | BRCA1+ | No cancer | Paternal cousin and | 7 | 11 |
| | | | | | aunt with breast cancer | | |
| F | 51 | Asian | Neg | Breast | Paternal grandmother | 7 | 9 |
| | | | | microcalcifications | with breast cancer | | |
| F | 48 | Caucasian | BRCA1− | No cancer | Paternal great aunt with | 9 | 9 |
| | | | | | breast cancer | | |
| F | 50 | Caucasian | BRCA1− | No cancer | Paternal great aunt | 7 | 18 |
| | | | | | with breast cancer | | |
| F | 47 | Caucasian | BRCA1− | No cancer | Paternal great aunt with | 9 | 9 |
| | | | | | breast cancer | | |
| F | 41 | Caucasian | BRCA1+ | No cancer | Sister with breast cancer | 7 | 7 |
| F | 56 | Caucasian | Neg | No cancer | Sister with breast cancer | 7 | 9 |
| F | 27 | N/K | BRCA2+ | No cancer | Two maternal aunts and | 7 | 7 |
| | | | | | mother with breast | | |
| | | | | | cancer | | |
| F | 44 | Caucasian | BRCA2+ | Benign breast | Two maternal aunts and | 9 | 10 |
| | | | | parenchyma | three paternal aunts | | |
| | | | | | with breast cancer | | |
| F | 51 | N/K | Neg | No cancer | Two maternal aunts with | 9 | 9 |
| | | | | | breast cancer | | |
| F | 30 | Caucasian | Neg | No breast cancer | Mother with bilateral | 7 | 8 |
| | | | | | breast cancer and | | |
| | | | | | ovarian ca, maternal | | |
| | | | | | grandmother with breast | | |
| | | | | | cancer | | |
| F | 30 | Caucasian | Neg | No breast cancer | Maternal and paternal | 7 | 8 |
| | | | | | grandmothers with | | |
| | | | | | breast cancer | | |
| F | 32 | Asian American | Neg | No breast cancer | Mother with breast and | 7 | 7 |
| | | | | | ovarian cancer, | | |
| | | | | | maternal aunt with | | |
| | | | | | breast cancer, hx of 1 | | |
| | | | | | breast bx | | |
| F | 70 | Caucasian | Neg | No breast cancer | Daughter with breast | 7 | 10 |
| | | | | | cancer, hx of 4 breast | | |
| | | | | | bx | | |
| F | 30 | Hispanic | Neg | No breast cancer | Mother and maternal | 10 | 12 |
| | | | | | aunt with breast cancer | | |
| F | 35 | Hispanic | Neg | No breast cancer | Mother with bilateral | 7 | 11 |
| | | | | | breast cancer, maternal | | |
| | | | | | aunt, maternal | | |
| | | | | | grandmother and | | |
| | | | | | paternal grandmother | | |
| | | | | | with breast cancer | | |
| F | 43 | Caucasian | Neg | No breast cancer | Mother and maternal | 7 | 7 |
| | | | | | grandmother with breast | | |
| | | | | | cancer, maternal uncle | | |
| | | | | | with colon cancer | | |
| F | 53 | Caucasian | Neg | No breast cancer | Two sisters and niece | 7 | 9 |
| | | | | | with breast cancer, hx of | | |
| | | | | | 1 breast bx | | |
| F | 49 | Caucasian | BRCA1+ | No breast cancer | 3 sisters, mother and | 7 | 7 |
| | | | | | maternal aunt with | | |
| | | | | | breast cancer; father | | |
| | | | | | with colon cancer, | | |
| | | | | | subject had 1 breast bx | | |
| F | 41 | Caucasian | Neg | No breast cancer | Mother, maternal | 7 | 9 |
| | | | | | grandmother and 2 | | |
| | | | | | sisters of the maternal | | |
| | | | | | grandfather had breast | | |
| | | | | | cancer, subject has had | | |
| | | | | | two breast bx | | |
| F | 41 | Caucasian | Neg | No breast cancer | Maternal aunt, maternal | 7 | 10 |
| | | | | | grandmother, and two | | |
| | | | | | maternal great aunts | | |
| | | | | | had breast cancer | | |
| F | 40 | Caucasian | Neg | No breast cancer | Sister and maternal aunt | 7 | 9 |
| | | | | | had breast cancer | | |
| F | 31 | Caucasian | Neg | No breast cancer | Mother with bilateral | 7 | 8 |
| | | | | | breast and ovarian | | |
| | | | | | cancer | | |
| M | 36 | Caucasian | Neg | No cancer | Maternal grandmother, | 9 | 9 |
| | | | | | maternal aunt, and | | |
| | | | | | mother with breast | | |
| | | | | | cancer | | |
| M | 73 | Caucasian | BRCA1+ | No cancer | Paternal great | 7 | 9 |
| | | | | | grandmother, paternal | | |
| | | | | | cousin, paternal aunt | | |
| | | | | | with breast cancer | | |
| M | 31 | Caucasian | Neg | No cancer | Positive for colon cancer | 9 | 9 |
| | | | | | in three paternal | | |
| | | | | | relatives | | |
| F | 49 | Caucasian | N/K | No cancer | Maternal grandfather | 7 | 7 |
| | | | | | had colon cancer | | |
| M | 27 | Ashkenazi/Polish | N/K | No cancer | Prostate cancer, breast | 7 | 9 |
| | | Jewish | | | cancer | | |
| M | 52 | Caucasian | N/K | No cancer | Grandmother had breast | 7 | 10 |
| | | | | | cancer | | |
| F | 35 | Caucasian | BRCA1+ | No breast cancer | Prophylactic mast. | 7 | 9 |

Breast cancer patients

| | | | | | | | |
|---|---|---|---|---|---|---|---|
| F | 67 | Black | Neg | Breast Cancer | N/K | 7 | 7 |
| F | 41 | Caucasian | Neg | Breast Cancer | N/K | 10 | 19 |
| F | 48 | African- | Neg | Breast Cancer | N/K | 7 | 19 |
| | | American | | | | | |
| F | 43 | Caucasian | Neg | Breast Cancer | Family hx of breast | 10 | 10 |
| | | | | | cancer | | |
| F | 49 | Caucasian | Neg | Breast Cancer | N/K | 9 | 10 |
| F | 32 | Black | Neg | Breast Cancer | Significant family hx of | 7 | 7 |
| | | | | | early onset colon cancer | | |
| | | | | | and sister with breast | | |
| | | | | | cancer | | |
| F | 70 | Black | Neg | Breast Cancer | N/K | 7 | 7 |
| F | 60 | Black | Neg | Breast Cancer | No breast cancer | 7 | 7 |
| F | 61 | East indian | Neg | Breast Cancer | N/K | 7 | 7 |
| F | 82 | Caucasian | Neg | Breast Cancer | N/K | 7 | 8 |
| F | N/K | N/K | Neg | Breast Cancer | N/K | 7 | 7 |
| F | 50 | Caucasian | Neg | Breast Cancer | Family hx of breast | 7 | 7 |
| | | | | | cancer | | |
| F | 49 | Black | Neg | Breast Cancer | N/K | 9 | 9 |
| F | 53 | Asian | Neg | Breast Cancer | N/K | 7 | 9 |
| F | 72 | Caucasian | Neg | Breast Cancer | N/K | 8 | 9 |
| F | 69 | Caucasian | Neg | Adenocarcinoma | N/K | 9 | 10 |
| F | 51 | Caucasian | Neg | Breast Cancer | N/K | 5 | 7 |
| F | N/K | N/K | Neg | Ductal carcinoma | N/K | 7 | 10 |
| F | 63 | Caucasian | Neg | Breast Cancer | N/K | 7 | 7 |
| F | 44 | Caucasian | BRCA2+ | Inv. Breast | N/K | 7 | 7 |
| | | | | Cancer | | | |
| F | 51 | Black | Neg | Breast Cancer | N/K | 10 | 17 |
| F | 77 | Caucasian | Neg | Breast Cancer | N/K | 7 | 9 |
| F | 44 | Caucasian | BRCA1+ | Breast Cancer | N/K | 7 | 9 |
| F | 41 | Caucasian | BRCA1+ | Breast Cancer | N/K | 7 | 9 |
| F | 47 | Caucasian | BRCA1+ | Breast Cancer | N/K | 7 | 16 |
| F | 42 | Caucasian | BRCA2+ | Breast Cancer | N/K | 9 | 12 |
| F | 34 | Caucasian | BRCA2+ | Breast Cancer | N/K | 7 | 17 |
| F | 36 | Caucasian | BRCA2+ | Breast Cancer | N/K | 10 | 10 |
| F | 41 | Caucasian | Neg | Breast Cancer | Family history of | 9 | 19 |
| | | | | | breast cancer | | |
| F | 41 | Caucasian | Neg | Breast Cancer | Family history of breast | 7 | 11 |
| | | | | | cancer | | |
| F | 44 | Caucasian | Neg | Breast Cancer | Family history of breast | 7 | 9 |
| | | | | | cancer | | |
| F | 51 | African-American | Neg | Metastatic breast | None | 7 | 7 |
| | | | | cancer | | | |
| F | 42 | Caucasian | Neg | Breast Cancer | None | 10 | 17 |
| F | 54 | Caucasian | Neg | Metastatic breast | Maternal grandmother, | 7 | 9 |
| | | | | | paternal great | | |
| | | | | | grandmother with breast | | |
| | | | | | cancer | | |
| F | 60 | African-American | Neg | Metastatic breast | None | 7 | 7 |
| | | | | cancer | | | |
| F | 42 | Caucasian | Neg | Metastatic breast | None | 7 | 10 |
| | | | | cancer | | | |
| F | 43 | Caucasian | Neg | Metastatic breast | None | 7 | 10 |
| | | | | cancer | | | |
| F | 46 | Caucasian | Neg | Metastatic breast | None | 10 | 10 |
| | | | | cancer | | | |
| F | 60 | Hispanic | Neg | Metastatic breast | None | 7 | 7 |
| | | | | cancer | | | |
| F | 63 | Caucasian | Neg | Metastatic breast | None | 9 | 9 |
| | | | | cancer | | | |
| F | 35 | Hispanic | Neg | Metastatic breast | None | 9 | 10 |
| | | | | cancer | | | |
| F | 63 | Caucasian | Neg | Metastatic breast | None | 9 | 9 |
| | | | | cancer | | | |
| F | 63 | Caucasian | Neg | Metastatic breast | None | 9 | 9 |
| | | | | cancer | | | |
| F | 46 | Caucasian | Neg | Metastatic breast | None | 10 | 10 |
| | | | | cancer | | | |
| F | 55 | African-American | Neg | Breast cancer | None | 7 | 8 |
| F | 46 | Caucasian | Neg | Metastatic breast | None | 10 | 10 |
| | | | | cancer | | | |
| F | 63 | Caucasian | Neg | Metastatic breast | None | 9 | 9 |
| | | | | cancer | | | |
| F | 46 | Caucasian | Neg | Metastatic breast | None | 10 | 10 |
| | | | | cancer | | | |
| F | 35 | Hispanic | Neg | Metastatic breast | None | 9 | 10 |
| | | | | cancer | | | |
| F | 63 | Caucasian | Neg | Metastatic breast | None | 9 | 9 |
| | | | | cancer | | | |
| F | 61 | Hispanic | Neg | Metastatic breast | None | 7 | 7 |
| | | | | cancer | | | |
| F | 46 | Caucasian | Neg | Metastatic breast | None | 10 | 10 |
| | | | | cancer | | | |
| F | 61 | Hispanic | Neg | Metastatic breast | None | 7 | 7 |
| | | | | cancer | | | |
| F | 46 | Caucasian | Neg | Metastatic breast | None | 10 | 10 |
| | | | | cancer | | | |
| F | 49 | Caucasian | Neg | Breast Cancer | Maternal aunt and | 11 | 18 |
| | | | | | mother with breast | | |
| | | | | | cancer | | |
| F | 53 | Caucasian | Neg | Breast Cancer | Maternal grandmother | 5 | 7 |
| | | | | | with breast cancer | | |
| F | 47 | Caucasian | Neg | Breast Cancer | None | 9 | 10 |
| F | 45 | Caucasian | Neg | Breast Cancer | Maternal great | 7 | 9 |
| | | | | | grandmother, maternal | | |
| | | | | | grandmother with breast | | |
| | | | | | cancer | | |
| F | 53 | African-American | Neg | Breast Cancer | None | 7 | 9 |
| F | 54 | Caucasian | Neg | Breast Cancer | None | 7 | 10 |
| F | 55 | Caucasian | BRCA1+ | Bilateral breast | Mother with breast | 7 | 7 |
| | | | | cancer | cancer | | |
| F | 65 | Caucasian | Neg | Breast Cancer | Mother with breast | 10 | 10 |
| | | | | | cancer | | |
| F | 54 | Caucasian | Neg | Breast Cancer | None | 7 | 7 |
| F | 54 | Caucasian | Neg | Breast Cancer | None | 7 | 7 |
| F | 64 | Caucasian | Neg | Breast Cancer | None | 7 | 7 |
| F | 54 | Hispanic | Neg | Breast Cancer | Mother and maternal | 7 | 12 |
| | | | | | cousin with breast | | |
| | | | | | cancer | | |
| F | 42 | Caucasian | Neg | Breast Cancer | Paternal great aunt with | 7 | 7 |
| | | | | | breast cancer | | |
| F | 54 | Caucasian | Neg | Breast Cancer | Half sister with breast | 7 | 9 |
| | | | | | cancer | | |
| F | 65 | Caucasian | Neg | Bilateral breast | None | 7 | 10 |
| | | | | cancer | | | |
| F | 52 | Caucasian | Neg | Breast Cancer | Maternal grandmother | 7 | 7 |
| | | | | | and mother with breast | | |
| | | | | | cancer | | |
| F | 61 | Caucasian | Neg | Breast Cancer | Sister with breast cancer | 10 | 11 |
| F | 74 | Caucasian | Neg | Breast Cancer | None | 7 | 9 |
| F | 52 | African-American | Neg | Breast Cancer | None | 7 | 9 |
| F | 59 | Caucasian | Neg | Breast Cancer | None | 10 | 10 |
| F | 59 | Asian | Neg | Breast Cancer | None | 9 | 11 |
| F | 69 | Caucasian | Neg | Breast Cancer | None | 7 | 9 |
| F | 50 | Caucasian | Neg | Breast Cancer | Paternal grandmother | 7 | 10 |
| | | | | | with breast cancer | | |
| F | 48 | Caucasian | Neg | Breast Cancer | None | 7 | 9 |
| F | 40 | African-American | Neg | Breast Cancer | Aunt with breast cancer | 7 | 9 |
| F | 50 | African-American | Neg | Breast Cancer | Mother with breast | 8 | 8 |
| | | | | | cancer | | |
| F | 34 | African-American | N/K | Metastatic breast | mother and 2 maternal | 7 | 7 |
| | | | | cancer | aunts with breast cancer | | |
| F | 53 | Caucasian | N/K | Metastatic | no family history of | 10 | 18 |
| | | | | breast cancer | cancer | | |
| F | 52 | African- | N/K | Metastatic | mother with throat | 7 | 17 |
| | | American | | breast cancer | cancer, aunt with | | |
| | | | | | pancreatic cancer, | | |
| | | | | | aunt with N/K cancer | | |
| F | 66 | African-American | N/K | Metastatic breast | mother with diabetes | 7 | 7 |
| | | | | cancer | and N/K cancer, sister | | |
| | | | | | with diabetes and | | |
| | | | | | ovarian cancer | | |
| F | 41 | Caucasian | N/K | Metastatic breast | no family history of | 7 | 11 |
| | | | | cancer | cancer | | |
| F | 60 | African-American | N/K | Metastatic breast | father with unspecified | 7 | 9 |
| | | | | cancer | GI cancer, maternal | | |
| | | | | | grandmother with breast | | |
| | | | | | cancer | | |
| F | 61 | African-American | N/K | Metastatic breast | no family history of | 7 | 8 |
| | | | | cancer | cancer | | |
| F | 50 | Caucasian | N/K | Metastatic breast | no family history of | 7 | 9 |
| | | | | cancer | cancer | | |
| F | 62 | Caucasian | N/K | Metastatic breast | no family history of | 10 | 12 |
| | | | | cancer | cancer | | |
| F | 58 | Caucasian | N/K | Metastatic breast | no family history of | 7 | 7 |
| | | | | cancer | cancer | | |
| F | 68 | Caucasian | N/K | Metastatic breast | father with cancer of N/K | 7 | 10 |
| | | | | cancer | primary, mother with | | |
| | | | | | Alzheimer's, paternal | | |
| | | | | | uncle with N/K cancer, | | |
| | | | | | maternal great- | | |
| | | | | | grandmother with | | |
| | | | | | ovarian cancer | | |
| F | 49 | African-American | N/K | Metastatic breast | N/K | 7 | 8 |
| | | | | cancer | | | |
| F | 50 | African-American | N/K | Metastatic breast | father with prostate | 7 | 7 |
| | | | | cancer | cancer | | |
| F | 44 | African-American | N/K | Breast Cancer | mother with breast | 7 | 10 |
| | | | | | cancer, father with lung | | |
| | | | | | cancer, maternal uncle | | |
| | | | | | with diabetes | | |
| F | 56 | Caucasian | N/K | Metastatic | mother with breast | 7 | 17 |
| | | | | breast cancer | cancer | | |
| F | 55 | African-American | N/K | Metastatic breast | undefined family history | 7 | 7 |
| | | | | cancer | of colon cancer | | |
| F | 62 | Asian | Neg | Metastatic breast | mother and sister with | 7 | 7 |
| | | | | cancer | breast cancer, maternal | | |
| | | | | | cousin with stomach | | |
| | | | | | cancer, maternal cancer | | |
| | | | | | with lymphoma | | |
| F | 47 | African-American | N/K | Metastatic breast | no family history of | 7 | 8 |
| | | | | cancer | cancer | | |
| F | 40 | N/K - listed as | Neg | Breast Cancer | breast cancer in mother | 7 | 7 |
| | | other | | | and paternal | | |
| | | | | | grandmother, father with | | |
| | | | | | leukemia | | |
| F | 46 | Caucasian | Neg | Bilateral breast | sister with breast | 12 | 16 |
| | | | | cancer | cancer, paternal uncle | | |
| | | | | | with mesothelioma, | | |
| | | | | | paternal grandfather | | |
| | | | | | with lung cancer | | |
| F | 71 | Caucasian | Neg | Breast Cancer | daughter with breast | 7 | 16 |
| | | | | | cancer and Paget's, | | |
| | | | | | father with colon | | |
| | | | | | cancer, paternal uncle | | |
| | | | | | with thyroid cancer, | | |
| | | | | | paternal cousin with | | |
| | | | | | breast cancer; | | |
| | | | | | paternal grandmother | | |
| | | | | | with leukemia, mother | | |
| | | | | | with colon and | | |
| | | | | | pancreatic cancer, | | |
| | | | | | maternal uncle with | | |
| | | | | | melanoma, maternal | | |
| | | | | | aunt N/K cancer, | | |
| | | | | | maternal aunt with | | |
| | | | | | breast cancer, | | |
| | | | | | maternal cousin with | | |
| | | | | | breast cancer; | | |
| | | | | | maternal grandmother | | |
| | | | | | with breast cancer, | | |
| | | | | | maternal grandfather | | |
| | | | | | with N/K cancer | | |
| F | 42 | African-American | BRCA2+ | Breast Cancer | maternal grandmother | 7 | 8 |
| | | | | | with colon cancer, | | |
| | | | | | mother with cervical | | |
| | | | | | cancer | | |
| F | 48 | Caucasian | N/K | Breast Cancer | no family history of | 7 | 11 |
| | | | | | cancer | | |
| F | 37 | Caucasian | BRCA2+ | Breast Cancer | maternal grandfather | 7 | 12 |
| | | | | | with prostate cancer | | |
| F | 78 | not given | Neg | Breast Cancer | sister with breast | 7 | 7 |
| | | | | | cancer, father with lung | | |
| | | | | | cancer, brother with | | |
| | | | | | leukemia, paternal | | |
| | | | | | grandmother with | | |
| | | | | | stomach cancer, | | |
| | | | | | paternal grandfather | | |
| | | | | | with prostate cancer | | |
| F | 36 | African-American | Neg | Breast Cancer | 2 paternal great aunts | 7 | 7 |
| | | | | | with breast cancer, | | |
| | | | | | paternal half-sister with | | |
| | | | | | leukemia | | |
| F | 35 | not given | BRCA2+ | Breast Cancer | paternal grandmother | 7 | 9 |
| | | | | | with breast, skin, and | | |
| | | | | | uterine cancer | | |
| F | 29 | Caucasian | N/K | Breast Cancer | maternal grandmother | 7 | 7 |
| | | | | | with breast, uterine, and | | |
| | | | | | gastric cancer; paternal | | |
| | | | | | uncle with lung cancer, | | |
| | | | | | paternal grandmother | | |
| | | | | | with brain cancer | | |
| F | 70 | not given | N/K | Ductal carcinoma | father with gastric | 7 | 7 |
| | | | | | cancer, mother with | | |
| | | | | | melanoma | | |
| F | 46 | Caucasian | N/K | Breast Cancer | mother with bone | 9 | 21 |
| | | | | | cancer | | |
| F | 74 | Caucasian | N/K | Breast Cancer | father with bile duct and | 7 | 7 |
| | | | | | gallbladder cancer, | | |
| | | | | | sister with breast | | |
| | | | | | cancer, maternal cousin | | |
| | | | | | with liver cancer | | |
| F | 36 | not given | Neg | Breast Cancer | maternal grandmother | 9 | 10 |
| | | | | | with colon cancer, great | | |
| | | | | | grandmother with breast | | |
| | | | | | cancer, paternal aunt | | |
| | | | | | with liver cancer, | | |
| | | | | | paternal aunt with non | | |
| | | | | | Hodgkins lymphoma, | | |
| | | | | | paternal grandmother | | |
| | | | | | with lung cancer | | |
| F | 40 | African-American | N/K | Metastatic | N/K | 7 | 7 |
| | | | | mucinous breast | | | |
| | | | | cancer | | | |
| F | 61 | Caucasian | Neg | Breast cancer | great grandmother, | 9 | 9 |
| | | | | | mother, and sister with | | |
| | | | | | breast cancer | | |
| F | 83 | Caucasian | N/K | Ductal | sister and maternal | 9 | 16 |
| | | | | carcinoma | aunt with breast | | |
| | | | | | cancer | | |
| F | 32 | Caucasian | BRCA2+ | Breast Cancer | paternal grandmother | 11 | 17 |
| | | | | | with lung cancer | | |
| F | 50 | Caucasian | N/K | Breast Cancer | 2 maternal aunts with | 7 | 10 |
| | | | | | breast cancer | | |
| F | 68 | African-American | N/K | Breast Cancer | no family history of | 7 | 7 |
| | | | | | cancer | | |
| F | 52 | Caucasian | N/K | Breast Cancer | paternal uncle with | 9 | 10 |
| | | | | | prostate cancer, | | |
| | | | | | paternal uncle with brain | | |
| | | | | | cancer | | |
| F | 58 | Caucasian | N/K | Breast Cancer | maternal aunt with | 9 | 10 |
| | | | | | stomach cancer | | |
| F | 35 | Caucasian | BRCA1 | Breast Cancer | mother with breast | 7 | 12 |
| | | | and | | cancer, maternal aunt | | |
| | | | BRCA2+ | | with ovarian cancer, | | |
| | | | | | father with prostate | | |
| | | | | | cancer, paternal aunt | | |
| | | | | | with kidney cancer | | |
| F | 52 | African-American | N/K | Breast Cancer | no family history of | 7 | 7 |
| | | | | | cancer | | |
| F | 58 | African-American | N/K | Invasive ductal | sister and paternal | 7 | 7 |
| | | | | carcinoma | grandmother with breast | | |
| | | | | | cancer | | |
| F | 38 | Caucasian | N/K | Invasive ductal | mother and sister with | 7 | 8 |
| | | | | carcinoma | breast cancer | | |
| F | 60 | Caucasian | Neg | Breast cancer | paternal first cousin with | 10 | 11 |
| | | | | | breast cancer, sister | | |
| | | | | | with glioblastoma, father | | |
| | | | | | and paternal uncle with | | |
| | | | | | prostate cancer | | |
| F | 66 | Caucasian | N/K | Invasive ductal | N/K | 7 | 10 |
| | | | | carcinoma | | | |
| F | 52 | Caucasian | N/K | Invasive ductal | daughter with non- | 7 | 9 |
| | | | | carcinoma | Hodkins lymphoma, | | |
| | | | | | distant cousin with | | |
| | | | | | leukemia | | |
| F | 42 | Caucasian | N/K | Invasive ductal | maternal great | 7 | 10 |
| | | | | carcinoma | grandmother and | | |
| | | | | | paternal aunt with | | |
| | | | | | breast cancer, maternal | | |
| | | | | | grandfather with | | |
| | | | | | prostate cancer | | |
| F | 42 | Caucasian | N/K | Invasive ductal | maternal great aunt and | 9 | 10 |
| | | | | carcinoma | paternal grandmother | | |
| | | | | | with breast cancer | | |
| F | 38 | Caucasian | N/K | Invasive ductal | paternal grandmother | 10 | 10 |
| | | | | carcinoma | with breast cancer | | |
| F | 54 | Caucasian | Neg | Breast Cancer | N/K | 10 | 21 |
| F | 51 | Caucasian | Neg | Breast Cancer | N/K | 7 | 10 |
| F | 81 | African-American | Neg | Breast Cancer | N/K | 7 | 7 |
| F | 52 | Caucasian | Neg | Breast Cancer | N/K | 7 | 8 |
| F | 53 | African-American | Neg | Breast Cancer | N/K | 7 | 8 |
| F | 64 | Caucasian | Neg | Breast Cancer | N/K | 7 | 7 |
| F | 43 | Caucasian | Neg | Breast cancer | N/K | | |
| F | | | | Basal Breast | | 9 | 9 |
| | | | | Cancer | | | |
| F | | | | Basal Breast | | 9 | 9 |
| | | | | Cancer | | | |
| F | | | | Basal Breast | | 9 | 17 |
| | | | | Cancer | | | |
| F | | | | Basal Breast | | 10 | 15 |
| | | | | Cancer | | | |
| F | | | | Basal Breast | | 7 | 8 |
| | | | | Cancer | | | |
| F | | | | Lum Breast | | 7 | 9 |
| | | | | Cancer | | | |
| F | | | | Lum Breast | | 9 | 16 |
| | | | | Cancer | | | |
| F | | | | Lum Breast | | 7 | 7 |
| | | | | Cancer | | | |
| F | | | | Lum Breast | | 10 | 10 |
| | | | | Cancer | | | |
| F | | | | Lum Breast | | 7 | 10 |
| | | | | Cancer | | | |

Colorectal cancer patients

| | | | | | | | |
|---|---|---|---|---|---|---|---|
| F | 43 | African- | N/K | Metastatic colon | Mother with breast and | 11 | 14 |
| | | American | | cancer | rectal cancer | | |
| F | 57 | Caucasian | N/K | Metastatic colon | None | 7 | 10 |
| | | | | cancer | | | |
| F | 74 | Caucasian | N/K | Uterine and colon | Niece with breast cancer | 7 | 8 |
| | | | | cancer | | | |
| F | 20 | African-American | N/K | Colon cancer | None | 7 | 11 |
| F | 57 | African-American | N/K | Invasive colonic | None | 11 | 11 |
| | | | | adenocarcinoma | | | |
| F | 87 | Caucasian | N/K | Invasive colonic | None | 7 | 7 |
| | | | | adenocarcinoma | | | |
| F | 61 | African-American | N/K | Invasive | Mother with colon | 7 | 11 |
| | | | | adenocarcinoma | cancer | | |
| F | 57 | Hispanic | N/K | Colonic | Three siblings and | 7 | 9 |
| | | | | adenocarcinoma | mother with colon | | |
| | | | | | cancer | | |
| F | 56 | African-American | N/K | Colonic | Brother with colon | 7 | 7 |
| | | | | adenocarcinoma | cancer | | |
| F | 72 | Caucasian | N/K | Invasive | None | 7 | 7 |
| | | | | mucinous | | | |
| | | | | adenocarcinoma | | | |
| F | 70 | African-American | N/K | Infiltrating | None | 10 | 12 |
| | | | | adenocarcinoma | | | |
| F | 60 | Caucasian | N/K | Invasive | Paternal aunt and father | 7 | 7 |
| | | | | adenocarcinoma | with colon cancer | | |
| F | 51 | African-American | N/K | Infiltrating | None | 9 | 9 |
| | | | | adenocarcinoma | | | |
| | | | | with focal | | | |
| | | | | mucinous areas | | | |
| F | 69 | Caucasian | N/K | adenocarcinoma | None | 9 | 10 |
| | | | | w/ mucin | | | |
| | | | | production | | | |
| F | 56 | African-American | N/K | Infiltrating | None | 7 | 7 |
| | | | | adenocarcinoma | | | |
| F | 64 | Caucasian | N/K | Invasive | Mother with colon | 9 | 10 |
| | | | | adenocarcinoma | polyps | | |
| F | 60 | Caucasian | N/K | Invasive | None | 9 | 9 |
| | | | | adenocarcinoma | | | |
| F | 76 | Caucasian | N/K | Invasive | None | 5 | 7 |
| | | | | adenocarcinoma | | | |
| F | 45 | Caucasian | N/K | Invasive colonic | Father with colon cancer | 7 | 7 |
| | | | | adenocarcinoma | | | |
| F | 77 | African-American | N/K | Invasive colonic | None | 7 | 8 |
| | | | | adenocarcinoma | | | |
| F | 78 | Caucasian | N/K | Infiltrating | None | 9 | 16 |
| | | | | colonic | | | |
| | | | | adenocarcinoma | | | |
| F | 68 | Caucasian | N/K | colonic | None | 9 | 9 |
| | | | | adenocarcinoma | | | |
| | | | | w/ signet ring | | | |
| | | | | features | | | |
| F | 71 | Hispanic | N/K | Infiltrating | None | 9 | 10 |
| | | | | adenocarcinoma | | | |
| F | 75 | Hispanic | N/K | Invasive | Two sisters with colon | 7 | 9 |
| | | | | adenocarcinoma | cancer | | |
| M | 63 | African-American | N/K | Invasive | None | 7 | 7 |
| | | | | adenocarcinoma | | | |
| M | 71 | African-American | N/K | infiltrating | None | 9 | 9 |
| | | | | adenocarcinoma | | | |
| M | 61 | African- | N/K | Invasive | None | 7 | 16 |
| | | American | | adenocarcinoma | | | |
| M | 68 | Caucasian | N/K | Colonic | None | 9 | 9 |
| | | | | adenocarcinoma | | | |
| M | 64 | Hispanic | N/K | Invasive colonic | None | 7 | 13 |
| | | | | adenocarcinoma | | | |
| M | 56 | Caucasian | N/K | Invasive colonic | None | 7 | 12 |
| | | | | adenocarcinoma | | | |
| M | 48 | Hispanic | N/K | Infiltrating colonic | None | 7 | 7 |
| | | | | adenocarcinoma | | | |
| M | 85 | Caucasian | N/K | Invasive colonic | None | 7 | 9 |
| | | | | adenocarcinoma | | | |
| M | 65 | African-American | N/K | Infiltrating | None | 7 | 10 |
| | | | | adenocarcinoma | | | |
| M | 71 | Caucasian | N/K | Infiltrating | None | 7 | 10 |
| | | | | adenocarcinoma | | | |
| M | 46 | Caucasian | N/K | Infiltrating | None | 9 | 9 |
| | | | | adenocarcinoma | | | |
| M | 53 | Caucasian | N/K | Infiltrating | None | 7 | 7 |
| | | | | adenocarcinoma | | | |
| M | 46 | Caucasian | N/K | Invasive | Grandmother and | 7 | 7 |
| | | | | mucinous | mother with breast | | |
| | | | | adenocarcinoma | cancer | | |
| M | 69 | Hispanic | N/K | Invasive colonic | Sister with breast | 7 | 7 |
| | | | | adenocarcinoma | cancer, sister with colon | | |
| | | | | | cancer | | |
| M | 72 | African-American | N/K | Invasive colonic | None | 7 | 7 |
| | | | | adenocarcinoma | | | |
| M | 49 | African-American | N/K | Invasive colonic | Sister with breast cancer | 7 | 8 |
| | | | | adenocarcinoma | | | |
| M | 41 | Caucasian | N/K | Invasive colonic | Aunt with breast cancer, | 9 | 10 |
| | | | | adenocarcinoma | paternal grandfather | | |
| | | | | | and father with colon | | |
| | | | | | cancer | | |
| M | 58 | African- | N/K | Invasive colonic | None | 7 | 19 |
| | | American | | adenocarcinoma | | | |
| M | 67 | African-American | N/K | Infiltrating colonic | None | 7 | 9 |
| | | | | adenocarcinoma | | | |
| | | | | w/ mucin | | | |
| | | | | production | | | |
| M | 72 | Caucasian | N/K | Invasive colonic | Sister with breast cancer | 7 | 7 |
| | | | | adenocarcinoma | | | |
| M | 43 | African-American | N/K | Colonic | None | 9 | 10 |
| | | | | adenocarcinoma | | | |
| M | 64 | African-American | N/K | Invasive | None | 7 | 7 |
| | | | | adenocarcinoma | | | |
| M | N/K | N/K | N/K | Colon cancer | N/K | 7 | 19 |
| M | N/K | N/K | N/K | Colon cancer | N/K | 5 | 8 |
| M | N/K | N/K | N/K | Colon cancer | N/K | 5 | 8 |
| N/K | N/K | N/K | N/K | Adenocarcinoma | None | 7 | 9 |
| N/K | N/K | N/K | N/K | Adenocarcinoma | None | 7 | 9 |

Patients with colon polyps

| | | | | | | | |
|---|---|---|---|---|---|---|---|
| F | 58 | Caucasian | N/K | Colon polyps | no known family | 7 | 15 |
| | | | | | history of cancer | | |
| F | 56 | N/K | N/K | Colon polyps | unspecified familly | 7 | 8 |
| | | | | | history of colon polyps | | |
| F | 52 | N/K | N/K | Colon polyps | uncle with colon cancer, | 7 | 7 |
| | | | | | aunt with breast cancer | | |
| F | 69 | Caucasian | N/K | Colon polyps | no family history of | 10 | 16 |
| | | | | | cancer | | |
| F | 59 | African American | N/K | Colon polyps | no known family history | 7 | 7 |
| | | | | | of cancer | | |
| F | 44 | Caucasian | N/K | Colon polyps | unspecifed family history | 10 | 10 |
| | | | | | of stomach cancer | | |
| F | 32 | African American | N/K | Colon polyps | unspecified family | 7 | 7 |
| | | | | | history of colon cancer | | |
| F | 68 | African American | N/K | Colon polyps | no known family history | 10 | 10 |
| | | | | | of cancer | | |
| F | 59 | Caucasian | N/K | Colon polyps | no known family history | 7 | 10 |
| | | | | | of cancer | | |
| F | 54 | Caucasian | N/K | Colon polyps | unspecified family | 11 | 11 |
| | | | | | history of colon cancer | | |
| F | 61 | Caucasian | N/K | Colon polyps | brother and neice with | 9 | 11 |
| | | | | | colon cancer | | |
| F | 63 | Caucasian | N/K | Colon polyps | mother with colon | 7 | 9 |
| | | | | | cancer | | |
| F | 42 | African American | N/K | Colon polyps | unspecified family | 7 | 10 |
| | | | | | history of cancer | | |
| F | 56 | African American | N/K | Colon polyps | no family history of | 8 | 9 |
| | | | | | cancer | | |
| F | 61 | Caucasian | N/K | Colon polyps | sister with colon cancer | 9 | 9 |
| F | 68 | Hispanic | N/K | Colon polyps | no known family history | 7 | 9 |
| | | | | | of cancer | | |
| F | 58 | Caucasian | N/K | Colon polyps | mom with kidney cancer | 7 | 9 |
| F | 53 | Hispanic | N/K | Colon polyps | N/K | 9 | 10 |
| F | 85 | African American | N/K | Colon polyps | N/K | 7 | 8 |
| F | 60 | African | N/K | Colon polyps | no family history of | 7 | 15 |
| | | American | | | cancer | | |
| F | 50 | African American | N/K | Colon polyps | no known family history | 7 | 9 |
| | | | | | of cancer | | |
| F | 66 | African American | N/K | Colon polyps | no known family history | 8 | 10 |
| | | | | | of cancer | | |
| F | 53 | Hispanic | N/K | Colon polyps | no known family history | 7 | 12 |
| | | | | | of cancer | | |
| F | 63 | Caucasian | N/K | Colon polyps | father and grandfather | 8 | 10 |
| | | | | | with colon cancer, | | |
| | | | | | paturnal aunt with | | |
| | | | | | kidney cancer, maternal | | |
| | | | | | aunt with ovarian cancer | | |
| F | 76 | African American | N/K | Colon polyps | mother with colon | 7 | 9 |
| | | | | | cancer | | |
| F | 55 | African American | N/K | Colon polyps | no known family history | 7 | 8 |
| | | | | | of cancer | | |
| F | 27 | Hispanic | N/K | Colon polyps | no family history of | 12 | 12 |
| | | | | | cancer | | |
| F | 51 | Hispanic | N/K | Colon polyps | no known family history | 7 | 7 |
| | | | | | of cancer | | |
| F | 64 | Hispanic | N/K | Colon polyps | father with stomach | 7 | 9 |
| | | | | | cancer, two sisters with | | |
| | | | | | colon polyps, | | |
| | | | | | unspecified relative with | | |
| | | | | | unspecified cancer | | |
| F | 56 | Caucasian | N/K | Colon polyps | grandmother with colon | 7 | 9 |
| | | | | | cancer, sister with | | |
| | | | | | breast cancer, mother | | |
| | | | | | with ovarian cancer | | |
| F | 54 | Caucasian | N/K | Colon polyps | no known family history | 7 | 11 |
| | | | | | of cancer | | |
| F | 52 | African American | N/K | Colon polyps | no known family history | 7 | 10 |
| | | | | | of cancer | | |
| F | 46 | Caucasian | N/K | Colon polyps | no known family history | 7 | 9 |
| | | | | | of cancer | | |
| F | 67 | African American | N/K | Colon polyps | no known family history | 7 | 8 |
| | | | | | of cancer | | |
| F | 59 | Caucasian | N/K | Colon polyps | no known family history | 5 | 7 |
| | | | | | of cancer | | |
| F | 61 | African | N/K | Colon polyps | no known family | 7 | 14 |
| | | American | | | history of cancer | | |
| F | 70 | African American | N/K | Colon polyps | no known family history | 7 | 8 |
| | | | | | of cancer | | |
| F | 63 | African American | N/K | Colon polyps | no known family history | 7 | 9 |
| | | | | | of cancer | | |
| F | 65 | Caucasian | N/K | Colon polyps | no known family history | 7 | 9 |
| | | | | | of cancer | | |
| F | 44 | Hispanic | N/K | Colon polyps | no known family history | 7 | 10 |
| | | | | | of cancer | | |
| F | 67 | African American | N/K | Colon polyps | no known family history | 7 | 7 |
| | | | | | of cancer | | |
| F | 55 | Caucasian | N/K | Colon polyps | no known family history | 7 | 9 |
| | | | | | of cancer | | |
| F | 50 | African American | N/K | Colon polyps | no known family history | 8 | 10 |
| | | | | | of cancer | | |
| F | 58 | Caucasian | N/K | Colon polyps | no known family history | 9 | 10 |
| | | | | | of cancer | | |
| F | 28 | Hispanic | N/K | Colon polyps | no known family history | 7 | 9 |
| | | | | | of cancer | | |
| F | 51 | Hispanic | N/K | Colon polyps | no known family history | 9 | 9 |
| | | | | | of cancer | | |
| F | 53 | African American | N/K | Colon polyps | no known family history | 7 | 7 |
| | | | | | of cancer | | |
| F | 57 | African American | N/K | Colon polyps | no known family history | 8 | 10 |
| | | | | | of cancer | | |
| F | 51 | Caucasian | N/K | Colon polyps | greatgrandfather with | 9 | 10 |
| | | | | | brain cancer, gradfather | | |
| | | | | | with stomach cancer | | |
| F | 58 | Hispanic | N/K | Colon polyps | unspecified relative | 9 | 14 |
| | | | | | with colon cancer, | | |
| | | | | | unspecified relative | | |
| | | | | | with breast cancer | | |
| F | 37 | Hispanic | N/K | Colon polyps | no known family history | 7 | 7 |
| | | | | | of cancer | | |
| F | 61 | Caucasian | N/K | Colon polyps | no known family history | 7 | 10 |
| | | | | | of cancer | | |
| F | 60 | Caucasian | N/K | Colon polyps | brother and sister with | 7 | 9 |
| | | | | | colon cancer | | |

Lung cancer cell lines

| | | | | | | | |
|---|---|---|---|---|---|---|---|
| F | 38 | Caucasian | N/K | Lung cancer | N/K | 7 | 9 |
| F | 46 | Caucasian | N/K | Lung cancer | N/K | 9 | 9 |
| F | 45 | Caucasian | N/K | SCLC | N/K | 7 | 11 |
| F | 54 | Caucasian | N/K | Lung cancer | N/K | 7 | 7 |
| M | 58 | Caucasian | N/K | Lung cancer | N/K | 5 | 7 |
| M | 60 | Caucasian | N/K | Lung cancer | N/K | 7 | 9 |
| M | N/K | N/K | N/K | Lung cancer | N/K | 7 | 9 |
| M | 65 | Caucasian | N/K | Lung cancer | N/K | 7 | 9 |
| M | 57 | Caucasian | N/K | Lung cancer | N/K | 7 | 9 |
| M | 53 | Caucasian | N/K | Lung cancer | N/K | 7 | 11 |
| M | 62 | Caucasian | N/K | Lung cancer | N/K | 8 | 9 |
| M | 59 | Black | N/K | Lung cancer | N/K | 7 | 7 |
| M | 55 | Caucasian | N/K | Lung cancer | N/K | 7 | 11 |
| M | 42 | Caucasian | N/K | Lung cancer | N/K | 9 | 9 |
| M | 54 | Caucasian | N/K | Lung cancer | N/K | 5 | 10 |
| M | 58 | Caucasian | N/K | Lung cancer | N/K | 7 | 10 |
| M | 56 | Black | N/K | Lung cancer | N/K | 9 | 10 |
| M | 69 | Caucasian | N/K | Lung cancer | N/K | 10 | 10 |
| M | 36 | Black | N/K | Lung cancer | N/K | 7 | 8 |
| M | 65 | Caucasian | N/K | Large cell | N/K | 7 | 15 |
| | | | | carcinoma | | | |
| M | N/K | Caucasian | N/K | Lung cancer | N/K | 7 | 9 |
| M | 67 | Caucasian | N/K | Lung cancer | N/K | 10 | 10 |

N/K = not known;
“No cancer” = no known/reported family hx of breast, ovarian, or colon cancer (1° or 2° family members).
Carriers of long (13+ copies AAAG) are indicated in bold red font.

TABLE 6
Comparisons of allelic frequencies for the AAAG repeat motif located
in the 5′ UTR of ERR-γ, grouped by race/ethnicity

| | Non-carriers | Carriers | Totals | Incidence |
|---|---|---|---|---|
| Caucasian/White | | | | |
| Healthy volunteers | | | | |
| No BC family hx | 41 | 3 | 44 | 6.8% |
| BC family hx | 32 | 3 | 35 | 8.6% |
| Breast cancer patients | 73 | 15 | 88 | 17.0% |
| Colorectal cancer patients | 20 | 1 | 21 | 4.8% |
| Patients with colorectal | 18 | 2 | 20 | 10.0% |
| polyps | | | | |
| Lung cancer cell lines | 17 | 1 | 18 | 5.6% |
| Totals | 201 | 25 | 226 | 11.1% |
| African/African-American/ | | | | |
| Black | | | | |
| Healthy volunteers | | | | |
| No BC family hx | 12 | 0 | 12 | 0.0% |
| BC family hx | 3 | 1 | 4 | 25.0% |
| Breast cancer patients | 29 | 3 | 32 | 9.4% |
| Colorectal cancer patients | 16 | 3 | 19 | 15.8% |
| Patients with colorectal | 18 | 2 | 20 | 10.0% |
| polyps | | | | |
| Lung cancer cell lines | 3 | 0 | 3 | 0.0% |
| Totals | 81 | 9 | 90 | 10.0% |
| Hispanic | | | | |
| Healthy volunteers | | | | |
| No BC family hx | 13 | 1 | 14 | 7.1% |
| BC family hx | 3 | 0 | 3 | 0.0% |
| Breast cancer patients | 6 | 0 | 6 | 0.0% |
| Colorectal cancer patients | 5 | 1 | 6 | 16.7% |
| Patients with colorectal | 10 | 1 | 11 | 9.1% |
| polyps | | | | |
| Lung cancer cell lines | 10 | 0 | 10 | 0.0% |
| Totals | 47 | 3 | 50 | 6.0% |

TABLE 7
Small Panel Used to Screen Individual Loci for Polymorphisms

| Sample ID | Sex | Race/Species | Tissue | Description |
|---|---|---|---|---|
| N7 | M | Caucasian | Blood | Cancer-free volunteer |
| N8 | F | Other | Blood | Cancer-free volunteer |
| N9 | F | Chinese | Blood | Cancer-free volunteer |
| N10 | F | African American | Blood | Cancer-free volunteer |
| N11 | F | Caucasian | Blood | Cancer-free volunteer |
| N12 | F | South East Asian | Blood | Coriell diversity sample (NA17083) |
| N13 | M | South East Asian | Blood | Coriell diversity sample (NA17085) |
| N14 | M | African American | Blood | Coriell diversity sample (NA17109) |
| N15 | F | African American | Blood | Coriell diversity sample (NA17112) |
| N16 | M | Caucasian | Blood | Coriell diversity sample (NA17241) |
| N17 | F | Caucasian | Blood | Coriell diversity sample (NA18006) |
| Mouse | M | Mus musculus | Blood | House mouse |
| P1320 | M | Pan troglodytes | Blood | Chimpanzee |
| P372 | M | Pan troglodytes | Blood | Chimpanzee |
| PR0053 | M | Gorilla gorilla | Blood | Lowland Gorilla |
| PR00107 | M | Gorilla gorilla | Blood | Lowland Gorilla |
| PR00253 | M | Pongo pygmaeus | Blood | Sumatran Orangutan |
| PR00002 | M | Pongo pygmaeus | Blood | Borneo Orangutan |
| HCC1008 | F | African American | Breast | TNM stage IIA, grade 3 metastatic |
| | | | | carcinoma |
| HCC1007BL | F | African American | Blood | Matched blood cell line |

Notes:
A dash (“—”) indicates that the information was not available. See Supplementary Table 1 for additional sample used in the panel, which included a total of 42 samples.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Cancer Facts and Figures 2009. (American Cancer Society, Atlanta).
2. Ideker, T. et al. Integrated genomic and proteomic analyses of a systematically perturbed metabolic network. Science 292, 929-34 (2001).
3. Beske, O. E. & Goldbard, S. High-throughput cell analysis using multiplexed array technologies. Drug Discov Today 7, S131-5 (2002).
4. Abd El-Rehim, D. M. et al. High-throughput protein expression analysis using tissue microarray technology of a large well-characterised series identifies biologically distinct classes of breast cancer confirming recent cDNA expression analyses. Int J Cancer 116, 340-50 (2005).
5. Ross, J. S. et al. The Her-2/neu gene and protein in breast cancer 2003: biomarker and target of therapy. Oncologist 8, 307-25 (2003).
6. Vogel, C. L. et al. Efficacy and safety of trastuzumab as a single agent in first-line treatment of HER2-overexpressing metastatic breast cancer. J Clin Oncol 20, 719-26 (2002).
7. Slamon, D. J. et al. Use of chemotherapy plus a monoclonal antibody against HER2 for metastatic breast cancer that overexpresses HER2. N Engl J Med 344, 783-92 (2001).
8. Esteva, F. J. et al. Phase II study of weekly docetaxel and trastuzumab for patients with HER-2-overexpressing metastatic breast cancer. J Clin Oncol 20, 1800-8 (2002).
9. Viani, G. A., Afonso, S. L., Stefano, E. J., De Fendi, L. I. & Soares, F. V. Adjuvant trastuzumab in the treatment of her-2-positive early breast cancer: a meta-analysis of published randomized trials. BMC Cancer 7, 153 (2007).
10. Forgacs, E. et al. Searching for microsatellite mutations in coding regions in lung, breast, ovarian and colorectal cancers. Oncogene 20, 1005-9 (2001).
11. Woerner, S. M. et al. Systematic identification of genes with coding microsatellites mutated in DNA mismatch repair-deficient cancer cells. Int J Cancer 93, 12-9 (2001).
12. Ellegren, H. Microsatellites: simple sequences with complex evolution. Nat Rev Genet. 5, 435-45 (2004).
13. Rubinsztein, D. C. et al. Sequence variation and size ranges of CAG repeats in the Machado-Joseph disease, spinocerebellar ataxia type 1 and androgen receptor genes. Hum Mol Genet. 4, 1585-90 (1995).
14. Fujisawa, T. et al. Length rather than a specific allele of dinucleotide repeat in the 5′ upstream region of the aldose reductase gene is associated with diabetic retinopathy. Diabet Med 16, 1044-7 (1999).
15. Laidlaw, J. et al. Elevated basal slippage mutation rates among the Canidae. J Hered 98, 452-60 (2007).
16. Girard, L., Zochbauer-Muller, S., Virmani, A. K., Gazdar, A. F. & Minna, J. D. Genome-wide allelotyping of lung cancer identifies new regions of allelic loss, differences between small cell lung cancer and non-small cell lung cancer, and loci clustering. Cancer Res 60, 4894-906 (2000).
17. Wistuba, I I et al. High resolution chromosome 3p allelotyping of human lung cancer and preneoplastic/preinvasive bronchial epithelium reveals multiple, discontinuous sites of 3p allele loss and three regions of frequent breakpoints. Cancer Res 60, 1949-60 (2000).
18. Jiricny, J. The multifaceted mismatch-repair system. Nat Rev Mol Cell Biol 7, 335-46 (2006).
19. Imai, K. & Yamamoto, H. Carcinogenesis and microsatellite instability: the interrelationship between genetics and epigenetics. Carcinogenesis 29, 673-80 (2008).
20. Riccio, A. et al. The DNA repair gene MBD4 (MED1) is mutated in human carcinomas with microsatellite instability. Nat Genet. 23, 266-8 (1999).
21. Tassone, F., Hagerman, R. J., Chamberlain, W. D. & Hagerman, P. J. Transcription of the FMR1 gene in individuals with fragile X syndrome. Am J Med Genet. 97, 195-203 (2000).
22. Bontekoe, C. J. et al. Instability of a (CGG)98 repeat in the Fmr1 promoter. Hum Mol Genet. 10, 1693-9 (2001).
23. Di Marco, S., Hel, Z., Lachance, C., Furneaux, H. & Radzioch, D. Polymorphism in the 3′-untranslated region of TNFalpha mRNA impairs binding of the post-transcriptional regulatory protein HuR to TNFalpha mRNA. Nucleic Acids Res 29, 863-71 (2001).
24. Fondon, J. W., 3rd & Garner, H. R. Molecular origins of rapid and continuous morphological evolution. Proc Natl Acad Sci USA 101, 18058-63 (2004).
25. Perou, C. M. et al. Molecular portraits of human breast tumours. Nature 406, 747-52 (2000).
26. Campeau, P. M., Foulkes, W. D. & Tischkowitz, M. D. Hereditary breast cancer: new genetic developments, new therapeutic avenues. Hum Genet. 124, 31-42 (2008).
27. Ionov, Y., Matsui, S. & Cowell, J. K. A role for p300/CREB binding protein genes in promoting cancer progression in colon cancer cell lines with microsatellite instability. Proc Natl Acad Sci USA 101, 1273-8 (2004).
28. Bacher, J. W. et al. Development of a fluorescent multiplex assay for detection of MSI-High tumors. Dis Markers 20, 237-50 (2004).
29. Fondon, J. W., 3rd et al. Computerized polymorphic marker identification: experimental validation and a predicted human polymorphism catalog. Proc Natl Acad Sci USA 95, 7514-9 (1998).
30. Berrieman, H. K. et al. Chromosomal analysis of non-small-cell lung cancer by multicolour fluorescent in situ hybridisation. Br J Cancer 90, 900-5 (2004).
31. Hong, H., Yang, L. & Stallcup, M. R. Hormone-independent transcriptional activation and coactivator binding by novel orphan nuclear receptor ERR3. J Biol Chem 274, 22618-26 (1999).
32. Ariazi, E. A., Clark, G. M. & Mertz, J. E. Estrogen-related receptor alpha and estrogen-related receptor gamma associate with unfavorable and favorable biomarkers, respectively, in human breast cancer. Cancer Res 62, 6510-8 (2002).
33. Riggins, R. B. et al. ERRgamma mediates tamoxifen resistance in novel models of invasive lobular breast cancer. Cancer Res 68, 8908-17 (2008).
34. Scillitani, A., Jong, C., Wong, B. Y., Hendy, G. N. & Cole, D. E. A functional polymorphism in the PTHR1 promoter region is associated with adult height and BMD measured at the femoral neck in a large cohort of young caucasian women. Hum Genet. 119, 416-21 (2006).
35. Jatoi, I. & Anderson, W. F. Management of women who have a genetic predisposition for breast cancer. Surg Clin North Am 88, 845-61, vii-viii (2008).
36. Decker, L. L., Klaman, L. D. & Thorley-Lawson, D. A. Detection of the latent form of Epstein-Barr virus DNA in the peripheral blood of healthy individuals. J Virol 70, 3286-9 (1996).
37. Khan, G., Miyashita, E. M., Yang, B., Babcock, G. J. & Thorley-Lawson, D. A. Is EBV persistence in vivo a model for B cell homeostasis? Immunity 5, 173-9 (1996).
38. Wagner, H. J., Bein, G., Bitsch, A. & Kirchner, H. Detection and quantification of latently infected B lymphocytes in Epstein-Barr virus-seropositive, healthy individuals by polymerase chain reaction. J Clin Microbiol 30, 2826-9 (1992).
39. Ariazi, E. A. & Jordan, V. C. Estrogen-related receptors as emerging targets in cancer and metabolic disorders. Curr Top Med Chem 6, 203-15 (2006).
40. Hurtado, A. et al. Regulation of ERBB2 by oestrogen receptor-PAX2 determines response to tamoxifen. Nature 456, 663-6 (2008).
41. Fondon, J. W., 3rd & Garner, H. R. Detection of length-dependent effects of tandem repeat alleles by 3-D geometric decomposition of craniofacial variation. Dev Genes Evol 217, 79-85 (2007).
42. Malone, K. E. et al. BRCA1 mutations and breast cancer in the general population: analyses in women before age 35 years and in women before age 45 years with first-degree family history. Jama 279, 922-9 (1998).
43. King, M. C., Marks, J. H. & Mandell, J. B. Breast and ovarian cancer risks due to inherited mutations in BRCA1 and BRCA2. Science 302, 643-6 (2003).
44. Schwartz, G. F. et al. Proceedings of the international consensus conference on breast cancer risk, genetics, & risk management, April, 2007. Breast J 15, 4-16 (2009).
45. Bejerano, G. et al. Ultraconserved elements in the human genome. Science 304, 1321-5 (2004).
46. Boland, C. R. et al. A National Cancer Institute Workshop on Microsatellite Instability for cancer detection and familial predisposition: development of international criteria for the determination of microsatellite instability in colorectal cancer. Cancer Res 58, 5248-57 (1998).
47. Umar, A. et al. Revised Bethesda Guidelines for hereditary nonpolyposis colorectal cancer (Lynch syndrome) and microsatellite instability. J Natl Cancer Inst 96, 261-8 (2004).
48. Heinemeyer, T. et al. Databases on transcriptional regulation: TRANSFAC, TRRD and COMPEL. Nucleic Acids Res 26, 362-7 (1998).

What is claimed is:

1. A method of identifying an increase in microsatellite DNA from a genomic nucleic acid sample comprising:
obtaining a microsatellite profile from a sample suspected of comprising cancer cells;
comparing the microsatellite profile to a reference microsatellite profile from a reference genome; and
determining in increase in the number of microsatellite DNAs from the sample as compared to the reference genome, wherein an increase in microsatellite DNA indicates a pre-disposition to cancer and the microsatellites are upstream from the estrogen receptor-related gamma gene (ESRRG).

2. The method of claim 1, wherein the microsatellite is TTTC and its copy number is elevated in the sample.

3. The method of claim 1, wherein the sample is from a patient suspected of having a pre-disposition to breast, colon or lung cancer.

4. The method of claim 1, wherein the sample from tissue that is somatic, germline or suspected of comprising cancer.

5. The method of claim 1, further comprising the step of amplifying a nucleic acid segment upstream from the ESRRG gene, and determining the number of TTTC repeats in the 5′ UTR, wherein an increase in the TTTC repeats in the reference genome indicates a pre-disposition to cancer.

6. The method of claim 1, wherein the sample is a clinical sample.

7. A method of detecting exposure of cells to carcinogens or mutagens comprising:
obtaining a microsatellite profile from a genomic nucleic acid from a cell sample suspected of exposure to the carcinogen or mutagen;
comparing the microsatellite profile of the cell sample to a reference cellular microsatellite profile normal cell sample; and
determining an change in the number of microsatellite DNAs from the cell sample as compared to the normal cell sample, wherein an change in microsatellite DNA indicates exposure to the carcinogen or mutagen.

8. The method of claim 7, wherein the cell sample is a clinical sample.

9. The method of claim 7, wherein the microsatellite profile is obtained using a microarray that comprises at least 3, 5, 7, 10, 12, 15, 18, 20, 22 or 25, spots selected from ACCTGA, AAAGAC; AATTT; AATT; AATTAG; ATAATT; AAATTT; AAATTG; AAAATT; ACATTT; AAAACG; AAAACT; ACTTAC; AAAAAT; AAAAGT; AAT; AAAGTT; ATATA; AAATAT; AAAGAT; AATAAG; AATAGG; AAATAG; AAAATG; AACCTT; AATATT; AAAGGT; and AAAG.

10. The method of claim 7, further comprising the step of knocking-down or knocking-out one or more genes in the cell sample and determining the change in microsatellite profile to identity one or more microsatellite sequences and the one or more genes that are adjacent to the change in microsatellite copy number to identify a suspected link between the microsatellite copy number and the one or more genes.

11. The method of claim 7, wherein a change in the copy number of the ACCTGA microsatellite is indicative of exposure to a carcinogen or mutagen.

12. A method of identifying a microsatellite associated with a disease condition from a sample comprising:
determining whether one or more microsatellite sequences from the sample has increased upstream from the ESRRG as compared to the reference genome that comprise a change in the copy number of the microsatellite sequence.

13. The method of claim 12, wherein the sample is a clinical sample.

14. The method of claim 12, wherein the sample is from a patient suspected of having an infectious disease, cancer, auto-inflammatory disease, auto-immune disease, metabolic disease.

15. The method of claim 12, wherein the microsatellite profile is obtained using a microarray that comprises at least 3, 5, 7, 10, 12, 15, 18, 20, 22 or 25, spots selected from ACCTGA, AAAGAC; AATTT; AATT; AATTAG; ATAATT; AAATTT; AAATTG; AAAATT; ACATTT; AAAACG; AAAACT; ACTTAC; AAAAAT; AAAAGT; AAT; AAAGTT; ATATA; AAATAT; AAAGAT; AATAAG; AATAGG; AAATAG; AAAATG; AACCTT; AATATT; AAAGGT; and AAAG.

16. The method of claim 12, further comprising the step of knocking-down or knocking-out one or more genes in the cell sample and determining the change in microsatellite profile to identity one or more microsatellite sequences and the one or more genes that are adjacent to the change in microsatellite copy number to identify a suspected link between the microsatellite copy number and the one or more genes.

17. A method of identifying a patient with a predisposition to cancer comprising:
determining if there is an increase or decrease in microsatellite copy number upstream of the AAAG tandem repeat locus located in the 5′ UTR of the estrogen-related receptor gamma gene (ESRRG) in a patient sample, the patient having the disease condition, wherein an change in microsatellite copy-number indicates a pre-disposition to cancer.

18. The method of claim 17, wherein the sample is a clinical sample.

19. The method of claim 17, wherein the cancer is elected from breast and colon cancer.

20. A method of identifying the phylogeny of a sample comprising:
obtaining a microsatellite profile for the sample using a microarray that comprises 1-mers to 6-mers of: perfect repeats, single mismatches, double mismatches and single nucleotide deletions;
comparing the microsatellite profile to a microsatellite profile from a reference genome; and
determining the phylogeny of the sample based on a comparison of the microsatellite profile of the sample to the reference genome.

21. The method of claim 20, wherein the sample is an unknown animal sample.

22. The method of claim 20, wherein the sample is a forensic sample.


SEQUENCE LISTING (3 sequences)

SEQ ID NO: 1 - DNA, 60 bp, Artificial Sequence
  Synthetic Oligonucleotide.
gcaaagggacccacggtggaacaggagcaggagcaggagcgggaggggcaggagcaggag

SEQ ID NO: 2 - DNA, 20 bp, Artificial Sequence
  Synthetic Oligonucleotide.
acctaggagatagaggttgc

SEQ ID NO: 3 - DNA, 20 bp, Artificial Sequence
  Synthetic Oligonucleotide.
cttcttctgcactatcaggg